(19) United States
(12) Patent Application Publication
(10) Pub. No.: US 2005/0124614 A1
(43) Pub. Date: Jun. 9, 2005

(54) 3,4-Dihydroisoquinolin-1-one derivatives as inducers of apoptosis

(75) Inventors: Anthony R Gangloff, San Diego, CA (US); Joane Litvak, Oakland, CA (US); Keith Pararajasingham, So San Francisco, CA (US); David Sperandio, Mountain View, CA (US)

Correspondence Address: AXYS PHARMACEUTICALS, INC., 180 KIMBALL WAY, SOUTH SAN FRANCISCO, CA, 94080, US

(73) Assignee: AXYS PHARMACEUTICALS, INC., So. San Francisco, CA (US)

(21) Appl. No.: 10/485,380

(22) PCT Filed: Jul. 3, 2003
(86) PCT No.: PCT/US03/21102

Related U.S. Application Data
(60) Provisional application No. 60/394,094, filed on Jul. 3, 2002.

(51) Int. Cl. A61K 31/5377; A61K 31/4709; C07D 417/02; C07D 413/02; C07D 43/02

(52) U.S. Cl. 514/232.5; 514/256; 514/309; 544/126; 544/333; 546/141

(57) ABSTRACT

The present invention related to certain 3,4-dihydroisoquinolin-1-ones that are activators of caspases and inducers of apoptosis, pharmaceutical composition comprising these compounds, and method of treating cancer utilizing these compounds.

### CROSS-REFERENCE

The Applicants claim priority under 35 U.S.C. 119(e) to copending Provisional Application No. 60/394,094 filed on Jul. 3, 2002, the disclosure of which is incorporated herein by reference in its entirety.

### BACKGROUND OF THE INVENTION

1. Field of the Invention

The present invention relates to certain 3,4-dihydroisoquinolin-1-one derivatives that are activators of caspases and inducers of apoptosis, pharmaceutical composition comprising these compounds, and method of treating cancer utilizing these compounds. Methods of preparing these compounds are also disclosed.

2. State of the Art

Organisms eliminate unwanted cells by a process variously known as regulated cell death, programmed cell death or apoptosis. Such cell death occurs as a normal aspect of animal development as well as in tissue homeostasis and aging (Glucksmann, A., Biol. Rev. Cambridge Philos. Soc. 1951, 26, 59-86; Glucksmann, A., Archives de Biologie 1965, 76, 419437; Ellis, et al., Dev. 1991, 112, 591-603; Vaux, et al. Cell 1994, 76, 777-779). Apoptosis regulates cell number, facilitates morphogenesis, removes harmful or otherwise abnormal cells and eliminates cells that have already performed their function. Additionally, apoptosis occurs in response to various physiological stresses, such as hypoxia or ischemia (The General Hospital Corporation. Programmed Cell Death Genes and Proteins. PCT published application WO96/20721, Jan. 4, 1996).

There are a number of morphological changes shared by cells experiencing regulated cell death, including plasma and nuclear membrane blebbing, cell shrinkage (condensation of nucleoplasm and cytoplasm), organelle relocalization and compaction, chromatin condensation and production of apoptotic bodies (membrane enclosed particles containing intracellular material) (Orrenius, S., J. Internal Medicine 1995, 237, 529-536.

Apoptosis is achieved through an endogenous mechanism of cellular suicide (Wyllie, A. H. In Cell Death in Biology and Pathology; Bowen and Lockshin, Eds.; Chapman and Hall, 1991; pp. 9-34). A cell activates its internally encoded suicide program as a result of either internal or external signals. The suicide program is executed through the activation of a carefully regulated genetic program (Wyllie, et al., Int Rev. Cyt. 1980, 68, 251; Ellis, et al., Ann Rev. Cell Bio. 1991, 7, 663). Apoptotic cells and bodies are usually recognized and cleared by neighboring cells or macrophages before lysis. Because of this clearance mechanism, inflammation is not induced despite the clearance of great numbers of cells (Orrenius, S., J. Internal Medicine 1995, 237, 529-536).

A group of proteases is a key element in apoptosis (see, e.g., Thorneberry, Chemistry and Biology 1998, 5, R97-R103; Thornberry, British Med. Bull. 1996, 53, 478-490). Genetic studies in the nematode Caenorhabditis elegans revealed that apoptotic cell death involves at least fourteen genes, two of which are the pro-apoptotic (death-promoting) ced (for cell death abnormal) genes, ced-3 and ced-4. CED-3 is homologous to interleukin 1 beta-converting enzyme, a cysteine protease, which is now called caspase-1. Further extensive research revealed that the mammalian apoptosis system appears to involve a cascade of caspases, or a system that behaves like a cascade of caspases. At present, the caspase family of cysteine proteases comprises fourteen different members, and more may be discovered in the future. All known caspases are synthesized as zymogens that require cleavage at an aspartyl residue prior to forming the active enzyme. Thus, caspases are capable of activating other caspases in the manner of an amplifying cascade.

Apoptosis and caspases are thought to be crucial in the development of cancer (Apoptosis and Cancer Chemotherapy; Hickman and Dive, Eds.; Humana Press: 1999). There is mounting evidence that cancer cells, while containing caspases, lack parts of the molecular machinery that activate the caspase cascade. This makes the cancer cells lose their capacity to undergo cellular suicide and the cells become immortal, i.e., they become cancerous. Control points are known to exist in the apoptosis process that represent points for intervention leading to activation. These control points include the CED-9-BCL-like and CED-3-ICE-like gene family products, which are intrinsic proteins regulating the fate of a cell to survive or die, respectively, and executing part of the cell death process itself (see, Schmitt, et al., Biochem. Cell. Biol. 1997, 75, 301-314). BCL-like proteins include BCL-XL and BAX-alpha, which appear to function upstream of caspase activation. BCL-XL appears to prevent activation of the apoptotic protease cascade, whereas BAX-alpha accelerates activation of the apoptotic protease cascade.

Chemotherapeutic (anti-cancer) drugs can trigger cancer cells to undergo suicide by activation of the dormant caspase cascade. This may be a crucial aspect of the mode of action of most, if not all, known anticancer drugs (Los, et al., Blood 1997, 90, 3118-3129; Friesen, et al., Nat. Med. 1996, 2, 574). The mechanism of action of current antineoplastic drugs frequently involves an attack at specific phases of the cell cycle. The cell cycle refers to the stages through which cells normally progress during their lifetimes. Normally, cells exist in a resting phase termed G₀ During multiplication, cells progress to a stage in which DNA synthesis occurs, termed S. Later, cell division, or mitosis, occurs in a phase called M. Antineoplastic drugs such as cytosine arabinoside, hydroxyurea, 6-mercaptopurine, and methotrexate are S phase specific, whereas antineoplastic drugs such as vincristine, vinblastine, and paclitaxel are M phase specific. Many slow growing tumors, for example colon cancers, exist primarily in the G₀ phase, whereas rapidly proliferating normal tissues, for example bone marrow, exist primarily in the S or M phase. Thus, the possibility exists for the activation of the caspase cascade, although the exact mechanisms for doing so presently are not clear. Furthermore, insufficient activity of the caspase cascade and consequent apoptotic events are implicated in various types of cancer.

The development of caspase cascade activators and inducers of apoptosis is a highly desirable goal in the development of therapeutically effective antineoplastic agents. Moreover, since autoimmune disease and certain degenerative diseases also involve the proliferation of abnormal cells, therapeutic treatment for these diseases could be effected by enhancement of the apoptotic process through the administration of appropriate caspase cascade activators and inducers of apoptosis.

### SUMMARY OF THE INVENTION

In one aspect, this invention is directed to a compound of Formula I: 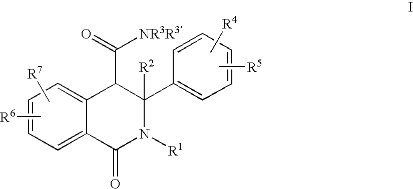
wherein:
R¹ is hydrogen, alkyl, cycloalkyl, cycloalkylalkyl, hydroxy, alkoxy, alkoxyalkyl, alkoxycarbonylalkyl, hydroxyalkyl, aryl, heteroaryl, aralkyl, heteroaralkyl, heterocycloalkylalkyl, or -alkylene-CONR⁸R⁹ where R⁸ is hydrogen, alkyl or alkoxyalkyl, and R⁹ is alkyl, optionally substituted aryl, optionally substituted aralkyl, alkoxyalkyl, optionally substituted heteroaryl, optionally substituted heteroaralkyl, heterocycloalkylalkyl, or saturated or unsaturated heterocycloaminoalkyl, or R⁸ and R⁹ together with the nitrogen atom to which they are attached form heterocycloamino;
R² is hydrogen or alkyl;
R³ is alkyl, alkoxy, hydroxy, haloalkyl, alkylthioalkyl, cycloalkyl, cycloalkylalkyl, hydroxyalkyl, alkoxyalkyl, alkoxycarbonylalkyl, carboxyalkyl, substituted carboxyalkyl, guanidino, heterocycloamino, aminoalkyl, substituted aminoalkyl, heterocycloaminoalkyl, alkylsulfonylalkyl, alkylsulfinylalkyl, heterocycloalkyl, optionally substituted aryl, optionally substituted heteroaryl, optionally substituted aralkyl, optionally substituted heteroaralkyl, aralkenyl, aryloxyalkyl, heteroaryloxyalkyl, -[(alkylene)-O]ₘ-(alkylene)-NH₂ (where m is 1, 2, or 3), heterocycloalkylalkyl, —C(O)R¹² where R12 is optionally substituted heteroaryl, or -(alkylene)-NR¹⁰R¹¹ where R¹⁰ and R¹¹ are independently selected from hydrogen, alkyl, optionally substituted aryl, optionally substituted heteroaryl, optionally substituted aralkyl, optionally substituted heteroaralkyl, or R¹⁰ and R¹¹ together with the nitrogen atom to which they are attached form saturated or unsaturated heterocycloamino;
R^{3′} is hydrogen or alkyl, or R³′ together with R³ and the nitrogen to which they are attached form heteroaryl or heterocycloamino;
R⁴ and R⁵ are independently of each other hydrogen, alkyl, halo, trifluoromethylthio, haloalkoxy, or haloalkyl; and
R⁶ and R⁷ are independently of each other hydrogen, alkyl, alkoxy, hydroxy, halo, haloalkyl, amino, alkylamino, dialkylamino, or acylamino; or
a pharmaceutically acceptable salt thereof;
provided that:
a) when R¹ is methyl, R², R³, R⁴, R⁵, R⁶ and R⁷ are hydrogen, then R3 is not —CH₂CO₂CH₃;
b) when R¹ is phenyl and R², R⁴, R⁵, R⁶, and R⁷ are hydrogen, then R³ and R^{3′} together with the nitrogen to which they are attached do not form pyrrolidinyl, piperidinyl, or morpholin-4-yl;
c) when R¹ is -alkylene-CONR⁸R⁹ and R² and R⁵ are hydrogen, then R^{3′} is hydrogen and R³ is aryloxyalkyl or substituted heterocycloalkyl (provided that substituted heterocycloamino is not substituted with alkoxyalkyl, alkyl, or hydroxyalkyl); or R³ and R^{3′} together with the nitrogen to which they are attached form substituted heterocycloamino (provided that the heterocycloamino is not substituted with hydroxy, hydroxyalkyl, or alkyl); or R⁹ is optionally substituted phenylalkyl.

Preferably a compound of Formula I, as represented by Ia: 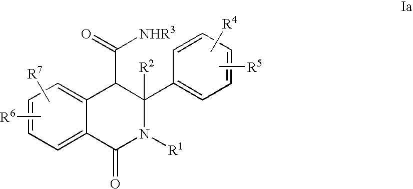
wherein:
R¹ is hydrogen, alkyl, cycloalkyl, cycloalkylalkyl, hydroxy, alkoxy, alkoxyalkyl, hydroxyalkyl, aryl, heteroaryl, aralkyl, heteroaralkyl, heterocycloalkylalkyl, or -alkylene-CONR⁸R⁹ where R⁸ is alkyl or alkoxyalkyl, and R⁹ is alkyl, optionally substituted aryl, optionally substituted aralkyl, alkoxyalkyl, optionally substituted heteroaryl, optionally substituted heteroaralkyl, heterocycloalkylalkyl, or saturated or unsaturated heterocycloaminoalkyl, or R⁸ and R⁹ together with the nitrogen atom to which they are attached form heterocycloamino;
R² is hydrogen or alkyl;
R³ is hydrogen, alkyl, alkoxy, hydroxy, haloalkyl, cycloalkyl, cycloalkylalkyl, alkylthioalkyl, hydroxyalkyl, alkoxyalkyl, alkoxycarbonylalkyl, carboxyalkyl, alkylsulfonylalkyl, heterocycloalkyl, optionally substituted aryl, optionally substituted heteroaryl, optionally substituted aralkyl, optionally substituted heteroaralkyl, aralkenyl, aryloxyalkyl, heteroaryloxyalkyl, or heterocycloalkylalkyl, or -alkylene)-NR¹⁰R¹¹ where R¹⁰ and R¹¹ are independently selected from hydrogen, alkyl, optionally substituted aryl, optionally substituted heteroaryl, optionally substituted aralkyl, optionally substituted heteroaralkyl, or R¹⁰ and R¹¹ together with the nitrogen atom to which they are attached form saturated or unsaturated heterocycloamino;
R⁴ is hydrogen, alkyl, halo, trifluoromethylthio, or haloalkyl;
R⁵ is alkyl, halo, trifluoromethylthio, or haloalkyl;
R⁶ and R⁷ are independently of each other hydrogen, alkyl, alkoxy, hydroxy, halo, haloalkyl, amino, alkylamino, dialkylamino, or acylamino; or
a pharmaceutically acceptable salt thereof;
provided that:
a) when R¹ is methyl, R², R^{3′}, R⁴, R⁵, R⁶ and R⁷ are hydrogen, then R³ is not —CH₂CO₂CH₃; and
b) when R¹ is phenyl and R², R⁴, R⁵, R⁶, and R⁷ are hydrogen, then R³ and R^{3′} together with the nitrogen to which they are attached do not form pyrrolidinyl, piperidinyl, or morpholin-4-yl.

In a second aspect, this invention is directed to a pharmaceutical composition comprising a therapeutically effective amount of a compound of Formula I or Ia and a pharmaceutically acceptable excipient.

In a third aspect, this invention is directed to a method of treating a disorder responsive to the induction of apoptosis in an animal suffering said disorder, comprising administering to said animal a pharmaceutical composition comprising a therapeutically effective amount of a compound of Formula I or a pharmaceutically acceptable salt thereof and a pharmaceutically acceptable excipient.

Preferably, the disorder is a cancer, autoimmune disease, rheumatoid arthritis, inflammatory bowel disease, or psoriasis. Preferably, the cancer is selected from the group consisting of Hodgkin's disease, non-Hodgkin's lymphoma, acute and chronic lymphocytic leukemias, multiple myeloma, neuroblastoma, breast carcinoma, ovarian carcinoma, lung carcinoma, Wilms' tumor, cervical carcinoma, testicular carcinoma, soft-tissue sarcoma, chronic lymphocytic leukemia, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head and neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer and prostatic carcinoma, and the animal is a human.

In a fourth aspect, this invention is directed to a method of treating cancer in an animal which method comprises administering to said animal a pharmaceutical composition comprising a therapeutically effective amount of a compound of Formula J or Ia and a pharmaceutically acceptable excipient in combination with radiation therapy and optionally in combination with one or more chemotherapeutic compound(s) independently selected from an estrogen receptor modulator, an androgen receptor modulator, retinoid receptor modulator, a cytotoxic agent, another antiproliferative agent, a prenyl-protein transferase inhibitor, an HMG-CoA reductase inhibitor, an HIV protease inhibitor, a reverse transcriptase inhibitor, or an angiogenesis inhibitor.

Preferably, the chemotherapeutic compound(s) is independently selected from Taxol®, Taxotere®, epothilone A, epothilone B, desoxyepothilone A, desoxyepothilone B or their derivatives; epidophyllotoxin; procarbazine; mitoxantrone; the mitomycins, discodermolide, podophyllotoxins, doxorubicin, carminomycin, daunorubicin, aminopterin, methotrexate, methopterin, dichloromethotrexate, mitomycin C, porfiromycin, Herceptin®, Rituxan®, 5-fluorouracil, 6-mercaptopurine, gemcitabine, cytosine arabinoside, colchicines, etoposide, etoposide phosphate, teniposide, melphalan, vinblastine, vincristine, vinorelbein, leurosidine, vindesine, leurosine, paclitaxel, estramustine, cisplatin, carboplatin, cyclophosphamide, bleomycin, tamoxifen, ifosamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, pyridobenzoindole derivatives, interferons, interleukins, capecitabine, and gefitinib.

In a fifth aspect, this invention is directed to a process of preparing a compound of Formula I comprising:
(a) reacting a compound of formula I where R¹ and R⁴-R⁷ are as defined in the Summary of the Invention 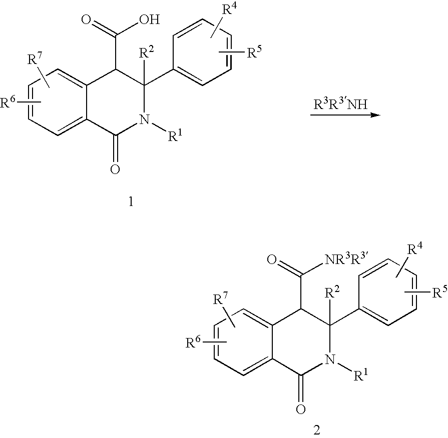
   with an amine of the formula R³R^{3′}NH where R³ and R^{3′} are as defined in the Summary of the Invention;
(b) optionally converting the compound obtained in step (a) above, to an acid addition salt;
(c) optionally converting a salt form of the compound obtained in step (a) above, to a free base;
(d) optionally separating individual isomers; and
(e) optionally modifying any of the R¹ and R⁴-R⁷ groups.

### DETAILED DESCRIPTION OF THE INVENTION

Definitions:

Unless otherwise stated, the following terms used in the specification and claims are defined for the purposes of this Application and have the following meanings:

“Acyl” means a radical —C(O)R where R is hydrogen, alkyl or trifluoromethyl, e.g., methylcarbonyl or trifluoromethylcarbonyl, and the like.

“Acylamino” means a radical —NHC(O)R where R is alkyl or trifluoromethyl, e.g., methylcarbonylamino or trifluoromethylcarbonylamino, and the like.

“Alkenylene” means a linear divalent hydrocarbon radical of two to six carbon atoms or a branched divalent hydrocarbon radical of three to six carbon atoms containing one or two double bonds e.g., ethenylene, propenylene, 1-methylpropenylene, butenylene, pentenylene, and the like.

“Alkoxy” means a radical —OR where R is alkyl as defined above, e.g., methoxy, ethoxy, propoxy, 2-propoxy, n-, iso-, or tert-butoxy, and the like.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxyethyl, 2-ethoxyethyl, 1-, 2-, or 3-methoxypropyl, and the like.

“Alkoxycarbonyl” means a radical —COOR where R is alkyl as defined above, e.g., methoxycarbonyl, ethoxycarbonyl, n-propoxycarbonyl, 2-propoxycarbonyl, n-, iso-, or tert-butoxycarbonyl, and the like.

“Alkoxycarbonylalkyl” means a radical -(alkylene)-COOR where R is alkyl as defined above, e.g., methoxycarbonylmethyl, ethoxycarbonylmethyl, and the like.

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like.

“Alkylamino” means a radical —NHR where R is alkyl as defined above, or an N-oxide derivative, or a protected derivative thereof, e.g., methylamino, ethylamino, n-, iso-propylamino, n-, iso-, tert-butylamino, methylamino-N-oxide, and the like.

“Alkylaminocarbonyl” means a radical —CONHR where R is an alkyl group as defined above, e.g, methylaminocarbonyl, ethylaminocarbonyl, and the like.

“Alkylene” means a linear saturated divalent hydrocarbon radical of one to six carbon atoms or a branched saturated divalent hydrocarbon radical of three to six carbon atoms, e.g., methylene, ethylene, propylene, 1-methylpropylene, 2-methylpropylene, butylene, pentylene, and the like.

“Alkylsulfinylalkyl” means a radical -(alkylene)-S(O)R where R is alkyl as defined herein, e.g., 2-(methylsulfinyl)ethyl, 3-(methylsulfinyl)propyl, or n-propylsulfinylmethyl, and the like.

“Alkylsulfonylalkyl” means a radical -(alkylene)-SO₂R where R is alkyl as defined above, e.g., methylsulfonylethyl, ethylsulfonylpropyl, (including all isomeric forms), and the like.

“Alkylthio” means a radical —SR where R is alkyl as defined above, e.g., methylthio, ethylthio, propylthio (including all isomeric forms), butylthio (including all isomeric forms), and the like.

“Alkylthioalkyl” means a radical -(alkylene)-SR where R is alkyl as defined above, e.g., methylthioetyl, ethylthiopropyl, (including all isomeric forms), and the like.

“Amino” means a radical —NH₂, or an N-oxide derivative, or a protected derivative thereof such as —NH→O, —NHBoc, —NHCBz, and the like.

“Aminoalkyl” means a linear monovalent hydrocarbon radical of one to six carbon atoms or a branched monovalent hydrocarbon radical of three to six carbons substituted with at least one, preferably one or two, —NRR′ where R and R′ are independently selected from hydrogen, alkyl, or —COR^{a} where R^{a} is alkyl, or an N-oxide derivative, or a protected derivative thereof e.g., aminomethyl, methylaminoethyl, 2-ethylamino-2-methylethyl, 1,3diaminopropyl, dimethylaminomethyl, diethylaminoethyl, acetylaminopropyl, and the like.

“Aminocarbonyl” means a radical —C(O)NH₂.

“Aralkenyl” means a radical -(alkenylene)-R where R is aryl as defined herein, e.g., phenylethenylene or naphtylpropyl-2-ene, and the like.

“Aralkyl” means a radical 4alkylene)-R where R is aryl as defined herein, e.g., benzyl, phenethyl, or napthylethyl, and the like.

“Aryl” means a monovalent monocyclic or bicyclic aromatic hydrocarbon radical of 6 to 12 ring atoms e.g., phenyl, naphthyl, or anthracenyl. The aryl ring may be optionally fused to a saturated or unsaturated heterocycloalkyl ring and optionally substituted on any of the rings with one, two, or three substituents independently selected from the group consisting of alkyl, alkoxy, alkylthio, haloalkyl, haloalkoxy, halo, hydroxy, dialkylamino, nitro, acyl, acylamino, alkoxycarbonyl, alkoxyalkyl, aminoalkyl, alkylaminoalkyl, dialkylaminoalkyl, dialkylaminocarbonyl, cyano, hydroxyalkyl, optionally substituted heteroaryl, or when two substituents are adjacent to each other they can combine to form methylenedioxy group or aryl is pentafluorophenyl.

“Aryloxyalkyl” means a radical -(alkylene)-OR where R is aryl as defined above, e.g., phenoxymethyl, phenoxyethyl, or napthyloxymethyl, and the like.

“Carboxyalkyl” means a radical -(alkylene)-COOH, e.g., carboxymethyl, carboxyethyl, 1-carboxy-2-methylbut-1-yl, or 1-carboxy-2-methylprop-1-yl, and the like.

“Cycloalkyl” means a cyclic saturated monovalent hydrocarbon radical of three to six carbon atoms, e.g., cyclopropyl, cyclobutyl, cyclopentyl, or cyclohexyl, and the like.

“Cycloalkylalkyl” means a -(alkylene)-R where R is cycloalkyl as defined above; e.g., cyclopropylmethyl, cyclobutylmethyl, cyclopentylethyl, or cyclohexylmethyl, and the like.

“Dialkylamino” means a radical —NRR′ where R and R′ are independently alkyl as defined above, or an N-oxide derivative, or a protected derivative thereof, e.g., dimethylamino, diethylamino, methylpropylamino, methylethylamino, n-, iso-, or tert-butylamino, and the like.

“Dialkylaminocarbonyl” means a radical —CONRR′ where R and R′ are independently an alkyl group as defined above e.g, dimethylaminocarbonyl or methylethylaminocarbonyl, and the like.

“Ethylenedioxy” means a radical —O—(CH₂)₂—O—.

“Halo” means fluoro, chloro, bromo, and iodo, preferably fluoro or chloro.

“Haloalkoxy” means a radical —OR where R is haloalkyl as defined herein, e.g., trifluoromethoxy or 2,2,2-trifluoroethoxy, and the like.

“Haloalkyl” means alkyl substituted with one or more halogen atoms, preferably one to three halogen atoms, preferably fluorine or chlorine, including those substituted with different halogens, e.g., —CH₂Cl, —CF₃, —CHF₂, or 2,2,3,3,3-pentafluoropropyl, and the like.

“Heteroaralkyl” means a radical -(alkylene)-R where R is heteroaryl as defined herein, e.g., furanylmethyl, pyridin-3-ylmethyl, 2-pyridin-4-ylethyl, thienylmethyl, or pyridin-2-ylmethyl, and the like.

“Heteroaryl” means a monovalent monocyclic or bicyclic aromatic radical of 5 to 10 ring atoms containing one or more, preferably one, two, or three ring heteroatoms selected from N, O, or S, SO₂, the remaining ring atoms being carbon. More specifically the term heteroaryl includes, but is not limited to, pyridyl, pyrrolyl, imidazolyl, thienyl, furanyl, indolyl, quinolyl, pyrazinyl, pyrimidinyl, pyridazinyl, oxazolyl, isoxazolyl, benzoxazolyl, quinolinyl, isoquinolinyl, benzopyranyl, thiadiazolyl, benzothiazolyl, [1,2,4]triazocin-3-yl, and thiazolyl, and the derivatives thereof, or N-oxide or a protected derivative thereof. The heteroaryl ring may be optionally substituted with one, two, or three substituents independently selected from the group consisting of alkyl, alkoxy, alkylthio, haloalkyl, haloalkoxy, halo, hydroxy, amino, dialkylamino, aminoalkyl, alkylaminoalkyl, dialkylaminoalkyl, nitro, acyl, thio, acylamino, alkoxycarbonyl, alkoxyalkyl, aminoalkyl, dialkylaminocarbonyl, cyano, hydroxyalkyl, or optionally substituted phenyl.

“Heteroaryloxyalkyl” means a radical -(alkylene)-OR where R is heteroaryl as defined above, e.g., furanyloxymethyl or pyridyloxymethyl, and the like.

“Heterocycloalkyl” means a saturated or unsaturated monovalent cyclic group of 3 to 10 ring atoms in which one, two, or three ring atoms are heteroatoms selected from N, O, or S(O)n, where n is an integer from 0 to 2, the remaining ring atoms being C where one or two carbon atoms can be optionally replaced by a carbonyl group. More specifically the term heterocycloalkyl includes, but is not limited to, 1H-pyrimidin-2,4-dione-5-yl, morpholino, tetrahydropyranyl, tetrahydrofuranyl, piperidinyl, thiomorpholino, and the like, and the derivatives thereof and N-oxide or a protected derivative thereof. The heterocycloalkyl ring may be optionally substituted, on any ring, with one, two, or three substituents independently selected from the group consisting of alkyl, alkoxy, alkoxyalkyl, alkylthio, haloalkyl, haloalkoxy, halo, hydroxy, amino, alkylamino, dialkylamino, nitro, acyl, acylamino, alkoxycarbonyl, aryloxyalkyl, aminoalkyl, aminocarbonyl, alkylaminocarbonyl, dialkylaminocarbonyl, carboxy, cyano, cycloalkyl, cycloalkylalkyl, optionally substituted phenyl, optionally substituted heteroaryl, optionally substituted phenylalkyl, optionally substituted heteroaralkyl, or hydroxyalkyl. When the term heterocycloalkyl is used, the group may be substituted or unsubstituted. When the term substituted heterocycloalkyl is used, the group must be substituted with at least one group selected from the substituents described above. More specifically, substituted heterocycloalkyl may include, but is not limited to 4-hydroxypiperidin-1-yl, N-benzylpiperidin-4-yl, or N-benzylpyrrolidinyl.

“Heterocycloalkylalkyl” means a radical -(alkylene)-R where R is heterocycloalkyl as defined above, e.g., tetrahydrofuran-2-ylmethyl, and the like.

“Heterocycloamino” means a saturated or unsaturated monovalent cyclic group of 3 to 10 ring atoms in which one, two, or three ring atoms are heteroatoms selected from N, O, or S(O)n, where n is an integer from 0 to 2 provided that at least one nitrogen atom is present, the remaining ring atoms being C where one or two carbon atoms can be optionally be replaced by a carbonyl group. The heterocycloamino may be optionally fused to aryl. More specifically the term heterocycloamino; includes, but is not limited to, pyrrolidino, piperidino, morpholino, piperazino, homopiperidino, or homopiperazino, and the like, and the derivatives thereof and N-oxide or a protected derivative thereof. The heterocycloamino group may be optionally substituted on any ring with one, two, or three substituents independently selected from the group consisting of alkyl, alkoxy, alkoxyalkyl, alkylthio, haloalkyl, haloalkoxy, halo, hydroxy, hydroxyalkyl, alkylaminosulfonyl, dialkylamino, nitro, acylamino, alkoxycarbonyl, —COR (where R is hydrogen, alkyl or haloalkyl), alkoxyalkyl, alkylaminocarbonyl, dialkylaminocarbonyl, cyano, optionally substituted phenyl, optionally substituted heteroaryl, optionally substituted phenylalkyl, optionally substituted heteroaralkyl, or ethylenedioxy. When the term heterocycloamino is used, the group may be substituted or unsubstituted. When the term substituted heterocycloamino is used, the group must be substituted with at least one group selected from the substituents described above. More specifically, substituted heterocycloamino may include, but is not limited to, 2,6-dimethylmorpholino, 4-acetylpiperazino, or 3-hydroxypyrrolidinyl, and the like.

“Heterocycloaminoalkyl” means -(alkylene)-R where R is heterocycloamino as defined herein. Representative examples include, but are not limited to, piperidin-4-ylmethyl, 2-morpholin-4-ylethyl, or piperazin-1-ylpropyl, and the like.

“Hydroxyalkyl” means a linear monovalent hydrocarbon radical of one to six carbon atoms or a branched monovalent hydrocarbon radical of three to six carbons substituted with one, two, or three hydroxy groups, provided that if two or three hydroxy groups are present any carbon atom does not contain more than one hydroxy. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, 1,3-dihydroxyprop-2-yl, 1,3-dihydroxy-2-methyl-prop-2-yl, or 1,3-dihydroxy-2-hydroxymethyl-prop-2-yl, and the like, preferably 2-hydroxyethyl, 2,3-dihydroxypropyl, or 1-(hydroxymethyl)-2-hydroxyethyl.

“Methylenedioxy” means a radical —O—CH₂—O—.

The present invention also includes the prodrugs of compounds of Formula I and Ia. The term prodrug is intended to represent covalently bonded carriers, which are capable of releasing the active ingredient of Formula I or Ia when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of Formula I and Ia include compounds wherein a hydroxy, amidino, guanidino, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula I and Ia), amides (e.g, trifluoroacetylamino, acetylamino, and the like), and the like. Prodrugs of compounds of Formula I and Ia are also within the scope of this invention.

The present invention also includes N-oxide derivatives and protected derivatives of compounds of Formula I and Ia. For example, when compounds of Formula I and Ia contain an oxidizable nitrogen atom, the nitrogen atom can be converted to an N-oxide by methods well known in the art. Also when compounds of Formula I and Ia contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. 1981, the disclosure of which is incorporated herein by reference in its entirety. The protected derivatives of compounds of Formula I and Ia can be prepared by methods well known in the art.

A “pharmaceutically acceptable salt” of a compound means a salt that is pharmaceutically acceptable and that possesses the desired pharmacological activity of the parent compound. Such salts include:
acid addition salts, formed with inorganic acids such as hydrochloric acid, hydrobromic acid, sulfric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or
salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The compounds of the present invention may have asymmetric centers. Compounds of the present invention containing an asymmetrically substituted atom may be isolated in optically active or racemic forms. It is well known in the art how to prepare optically active forms, such as by resolution of materials. All chiral, diastereomeric, racemic forms are within the scope of this invention. For example, in a compound of the present invention where only the C-3 and C-4 carbon atoms in the 3,4-dihydroisoquinolin-1-one ring are chiral, one can obtain two diastereomers of such compound i.e., compounds having cis or trans configurations at these substituent positions. All such diastereomers and mixtures of such diasteromers are within the scope of this invention. However, trans configuration is preferred.

Certain compounds of Formula I and Ia can exist as tautomers. All possible tautomers are within the scope of this invention. Additionally, as used herein the terms alkyl includes all the possible isomeric forms of said alkyl group albeit only a few examples are set forth. Furthermore, when the cyclic groups such as aryl, heteroaryl, heterocycloalkyl are substituted, they include all the positional isomers albeit only a few examples are set forth.

“Optional” or “optionally” means that the subsequently described event or circumstance may but need not occur, and that the description includes instances where the event or circumstance occurs and instances in which it does not. For example, “heterocycloalkyl group optionally mono- or di-substituted with an alkyl group” means that the alkyl may but need not be present, and the description includes situations where the heterocycloalkyl group is mono- or disubstituted with an alkyl group and situations where the heterocycloalkyl group is not substituted with the alkyl group.

“Optionally substituted aralkyl” means a radical -(alkylene)-R where R is optionally substituted aryl as defined herein, e.g., benzyl, phenethyl, or 4-methoxyphenylmethyl, and the like.

“Optionally substituted aryl” means a monovalent monocyclic or bicyclic aromatic hydrocarbon radical of 6 to 12 ring atoms e.g., phenyl, naphthyl or anthracenyl. The aryl ring may be optionally fused to a saturated or unsaturated heterocycloalkyl ring and optionally substituted on any of the rings with one, two, or three substituents independently selected from the group consisting of alkyl, alkoxy, alkylthio, haloalkyl, haloalkoxy, halo, hydroxy, amino, alkylamino, dialkylamino, nitro, acyl, acylamino, alkoxycarbonyl, carboxy, alkoxyalkyl, aminoalkyl, aminocarbonyl, alkylaminoalkyl, dialkylaminoalkyl, dialkylaminocarbonyl, cyano, hydroxyalkyl, optionally substituted heteroaryl, or when two substituents are adjacent to each other they can combine to form methylenedioxy group or aryl is pentafluorophenyl.

“Optionally substituted heteroaralkyl” means a -(alkylene)-R where R is optionally substituted heteroaryl ring as defined herein.

“Optionally substituted heteroaryl” means a heteroaryl ring as defined above which is optionally substituted with one, two, or three substituents independently selected from alkyl, halo, alkoxy, trifluoromethyl, trifluoromethoxy, amino, alkylamino, dialkylamino, hydroxy, cyano, nitro, aminocarbonyl, hydroxyalkyl, alkoxycarbonyl, thio, optionally substituted phenyl, or aminoalkyl. More specifically the term optionally substituted heteroaryl includes, but is not limited to, pyridyl, pyrrolyl, imidazolyl, thienyl, furanyl, indolyl, quinolyl, pyrazinyl, pyrimidinyl, pyridazinyl, oxazolyl, isooxazolyl, benzoxazolyl, quinolinyl, isoquinolinyl, benzopyranyl, and thiazolyl, and the derivatives thereof, or N-oxide or a protected derivative thereof

“Optionally substituted phenyl” means a phenyl ring optionally substituted with one, two, or three substituents independently selected from alkyl, halo, alkoxy, alkylthio, trifluoromethyl, trifluoromethoxy, amino, alkylamino, dialkylamino, hydroxy, cyano, nitro, methylenedioxy, aminocarbonyl, hydroxyalkyl, alkoxycarbonyl, aminoalkyl, or carboxy or optionally substituted with five fluorine atoms.

“Optionally substituted phenylalkyl” means a radical -(alkylene)-R where R is optionally substituted phenyl as defined above e.g., benzyl, phenylethyl, and the like.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Saturated heterocycloamino” means a saturated monovalent cyclic group of 3 to 10 ring atoms in which one, two, or three ring atoms are heteroatoms selected from N, O, or S(O)n, where n is an integer from 0 to 2 provided that at least one nitrogen atom is present, the remaining ring atoms being C where one or two carbon atoms can be optionally be replaced by a carbonyl group. The heterocycloamino may be optionally fused to aryl. More specifically the term heterocycloalkylamino; includes, but is not limited to, pyrrolidino, piperidino, morpholino, piperazino, homopiperidino, homopiperazino, and the like, and the derivatives thereof and N-oxide or a protected derivative thereof. The heterocycloalkyamino group may be optionally substituted on any ring with one, two, or three substituents independently selected from the group consisting of alkyl, alkoxy, alkoxyalkyl, alkylthio, haloalkyl, haloalkoxy, halo, hydroxy, hydroxyalkyl, amino, alkylamino, dialkylamino, nitro, acylamino, alkoxycarbonyl, alkoxyalkyl, aminoalkyl, aminocarbonyl, alkylaminocarbonyl, dialkylaminocarbonyl, carboxy, cyano, optionally substituted phenyl, optionally substituted heteroaryl, optionally substituted phenylalkyl, optionally substituted heteroaralkyl, hydroxyalkyl or ethylenedioxy.

“Saturated heterocycloaminoalkyl” means a radical -(alkylene)-R where R is saturated heterocycloamino as defined herein.

“Substituted aminoalkyl” means aminoalkyl as defind herein that is further substituted on the alkylene with aminocarbonyl, e.g., 5-amino-1-aminocarbonylpentyl or 5-amino-1-carboxypentyl, and the like.

“Substituted carboxyalkyl” means a radical -(alkylene)-COOH, where the alkylene, as defined herein, is substituted with one or two substituents independently selected from the group consisting of optionally substitued aryl, aminocarbonyl, or amino. More specifically the term substituted carboxyalkyl includes, but is not limited to, 3-aminocarbonyl-1-carboxypropyl or 2-phenyl-1-carboxyethyl, and the like.

“Treating” or “treatment” of a disease includes:
(1) preventing the disease, i.e. causing the clinical symptoms of the disease not to develop in a mammal that may be exposed to or predisposed to the disease but does not yet experience or display symptoms of the disease;
(2) inhibiting the disease, i.e., arresting or reducing the development of the disease or its clinical symptoms; or
(3) relieving the disease, i.e., causing regression of the disease or its clinical symptoms.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

A “therapeutically effective amount” means the amount of a compound of Formula I or Ia that, when administered to a mammal for treating a disease, is sufficient to effect such treatment for the disease. The “therapeutically effective amount” will vary depending on the compound, the disease and its severity and the age, weight, etc., of the mammal to be treated.

“Unsaturated heterocycloamino” means a monovalent cyclic group of 3 to 10 ring atoms in which one, two, or three ring atoms are heteroatoms selected from N, O, or S(O)n, where n is an integer from 0 to 2 provided that at least one nitrogen atom is present, the remaining ring atoms being C and which additionally contains one or two double bonds. The heterocycloamino group may be optionally substituted with alkyl, halo, alkoxy, or hydroxy. Examples include, but are not limited to, dihydropyrrole, tetrahydropyridine, tetrahydroazepine, tetrahydroisoquinoline, and the like.

“Unsaturated heterocycloaminoalkyl” means a radical -(alkylene)-R where R is unsaturated heterocycloamino as defined above.

### PREFERRED EMBODIMENTS

While the broadest definition of this invention is set forth in the Summary of the Invention, certain compounds of Formula I are preferred. For example:

A. Another preferred group of compounds is that wherein R¹ is hydrogen.

B. Another preferred group of compounds is that wherein R¹ is alkyl, more preferably methyl, ethyl, or 2-propyl, even more preferably methyl.

C. Another preferred group of compounds is that wherein R¹ is -alkylene-CONR⁸R⁹, where R⁸ and R⁹ together with the nitrogen atom to which they are attached form heterocycloamino, more preferably R¹ is 2-(piperidin-1-ylcarbonyl)ethyl, 2-(4-hydroxypiperidin-1-ylcarbonyl)ethyl, 2-(morpholin-4-ylcarbonyl)ethyl, 2-(4-acetylpiperazin-1-ylcarbonyl)ethyl, 2-(4-methylpiperidin-1-ylcarbonyl)ethyl, 2-(thiomorpholin-4-ylcarbonyl)ethyl, or 2-(4-formylpiperazin-1-ylcarbonyl)ethyl, even more preferably R¹ is 2-(4-hydroxypiperidin-1-ylcarbonyl)ethyl.

Within the above preferred groups A-C, a more preferred group of compounds is that wherein R² is hydrogen.

Within the above preferred groups A-C, another more preferred group of compounds is that wherein R² is alkyl, preferably methyl.

Within the above preferred and more preferred groups, an even more preferred group of compounds is that wherein:
R⁴ is hydrogen, methyl, chloro, bromo, trifluoromethylthio, trifluoromethoxy, or trifluoromethyl;
R⁵ is hydrogen, methyl, chloro, bromo, trifluoromethylthio, trifluoromethoxy, or trifluoromethyl, more preferably R⁴ and R⁵ are trifluoromethyl, or even more preferably R⁴ and R⁵ are trifluoromethyl located at the 3- and 5-position of the phenyl ring; and
R⁶ and R⁷ are independently of each other hydrogen, alkyl, alkoxy, hydroxy, halo, haloalkyl, amino, alkylamino, dialkylamino, or acylamino, preferably hydrogen, alkyl, alkoxy, or halo, more preferably, hydrogen, methyl, methoxy, hydroxy, chloro, fluoro, or amino, even more preferably, hydrogen, 6-methyl, 7-methyl, 6-methoxy, 7-methoxy, 6-chloro, or 7-chloro, particularly preferably hydrogen.

Within the above preferred, more preferred and even more preferred groups, particularly preferred group of compounds is that wherein:
R³ is alkyl, alkoxy, hydroxy, haloalkyl, alxylthioalkyl, cycloalkyl, cycloalkylalkyl, hydroxyalkyl, alkoxyalkyl, alkoxycarbonylalkyl, carboxyalkyl, substituted carboxyalkyl, guanidino, heterocycloamino, aminoalkyl, substituted aminoalkyl, heterocycloaminoalkyl, alkylsulfonylalkyl, alkylsulfinylalkyl, heterocycloalkyl, optionally substituted aryl, optionally substituted heteroaryl, optionally substituted aralkyl, optionally substituted heteroaralkyl, aralkenyl, aryloxyalkyl, heteroaryloxyalkyl, -[(alkylene)-O]ₘ-(alkylene)-NH₂ (where m is 1, 2, or 3), heterocycloalkylalkyl, —C(O)R¹² where R¹² is optionally substituted heteroaryl, or -(alkylene)-NR¹⁰R¹¹ where R¹⁰ and R¹¹ are independently selected from hydrogen, alkyl, optionally substituted aryl, optionally substituted heteroaryl, optionally substituted aralkyl, optionally substituted heteroaralkyl, or R¹⁰ and R¹¹ together with the nitrogen atom to which they are attached form saturated or unsaturated heterocycloamino,
preferably, 2-hydroxypyrid-6-yl, 2-chloropyrid-3-yl, 2-thio-[1,3,4]-thiadiazol-2-yl, 5,8-diphenyl-[1,2,4]triazocin-3-yl, 6-ethoxy-benzothiazol-2-yl, 6-fluoro-benzothiazol-2-yl, 3,5-dimethylisoxazol-4-yl, 5-methylisoxazol-3-ylmethyl, pyrimidin-2-yl, 3-methylpyrid-2-yl, 4-methylpyrid-2-yl, 5-methylpyrid-2-yl, 6-methylpyrid-2-yl, 4,6-dimethylpyrid-2-yl, 3-methylpyrid-4-yl, 2-methylpyrid-4-yl, 1,3-dimethylpyrazol-5-yl, 5-methylpyrazol-3-yl, 4-methylpyrimidin-2-yl, 4,6-dimethylpyrimidin-2-yl, 2,4-dimethylpyrimidin-6-yl, pyrazin-2-yl, pyrid-4-yl, pyrid-2-yl, pyrid-3-yl, pyrazol-3-yl, furan-2-ylmethyl, furan-2-ylcarbonyl, 5,6-dimethyl-[1,2,4]-triazin-3-yl, pyrimidin-4-yl, [1,3,4]-thiadiazol-2-yl, thiazol-2-yl, isoxazol-3-yl, cyclopentyl, 1H-pyrimidin-2,4-dione-5-yl, 2-methoxyethyl, cyclobutyl, cyclopropylmethyl, 3-hydroxyprop-2-yl, cyclohexylmethyl, pyrid-2-ylmethyl, pyrid-3-ylmethyl, pyrid-4-ylmethyl, pyrid-4-ylethyl, imidazol-4-ylethyl, thiophen-2-ylmethyl, cyclopentylmethyl, 2-hydroxypropyl, 3-hydroxypropyl, 2,3-dihydroxypropyl, 2-hydroxyethyl, 2-ethoxyethyl, 5-methylfuran-2-ylmethyl, cyclopropyl, cyclohexyl, 3-methoxypropyl, 1-hydroxy-4-methylpent-2-yl, 1-(furan-2-yl)ethyl, 5-(dimethylaminomethyl)furan-2-ylmethyl, 5-bromofuran-2-ylmethyl, 5-chlorofuran-2-methyl, 1,3-dihydroxyprop-2-yl, 1,3-dihydroxy-2-methylprop-2-yl, 3-hydroxy-2-methylprop-2-yl, 3-methoxyprop-2-yl, 1-tert-butyl-2-hydroxyethyl, 1-hydroxy-3-methylpent-2-yl, 1,3-dihydroxy-2-hydroxymethylprop-2-yl, 1,3-dihydroxybut-2-yl, 1,2-dimethylpyrrol-5-ylmethyl, 1-methylpyrrol-2-ylmethyl, imidazol-1-ylpropyl, furan-3-ylmethyl, 2,5-dimethylfuran-3-ylmethyl, 3-(methoxycarbonyl)furan-2-ylmethyl, 6-hydroxyhexyl, N-benzylpiperidin-4-yl, N-benzylpyrrolidin-3-yl, 2-phenyloxyethyl, benzyl, morpholin-4-yl, 2-(morpholin-4-yl)ethyl, 4,5-dihydrothiazol-2-yl, piperidin-4-yl, piperidin-4-ylmethyl, 2-methylpropyl, tert-butyl, methyl, tetrahydrofuran-2-ylmethyl, hydroxy, methoxy, ethyl, propyl, 2-fluoroethyl, 2,2,2-trifluoroethyl, 2,2,3,3,3-pentafluoropropyl, 2-methylthioethyl, —(CH₂)₂O(CH₂)₂O(CH₂)₂NH₂, 1-carboxy-3-methylbut-1-yl, 2-carboxyethyl, 3-aminocarbonyl-1-carboxypropyl, 1-carboxy-2-methylbutyl, carboxymethyl, 1-carboxy-2-methylpropyl, 2-phenyl-1-carboxyethyl, 2,3-dimethoxyphenylmethyl, 3,5-dimethoxyphenylmethyl, 3,4-difluorophenylmethyl, 2,4-difluorophenylmethyl, 4-fluorophenylmethyl, 3-difluoromethoxyphenylmethyl, 2,6-dimethoxyphenylmethyl, 2-(methylsulfinyl)ethyl, 2-(methylsulfonyl)ethyl, 2-hydroxyphenyl, 4-chloro-2-hydroxyphenyl, 2-amino-4-oxo-3H-pyrimidin-6-yl, 2-cyanophenyl, 5-amino-1-carboxypentyl, 5-amino-1-aminocarbonylpentyl, 2-(4-methoxyphenyl)ethyl, or guanidino,
more preferably, 4-methylpyrimidin-2-yl, 4,6-dimethylpyrimidin-2-yl, 2,4-dimethylpyrimidin-6-yl, pyrazin-2-yl, pyrid-4-yl, pyrid-2-yl, pyrid-3-yl, pyrazol-3-yl, furan-2-ylmethyl, furan-2-ylcarbonyl, 5,6-dimethyl-[1,2,4]-triazin-3-yl, pyrimidin-4-yl, [1,3,4]-thiadiazol-2-yl, thiazol-2-yl, isoxazol-3-yl, cyclopentyl, 1H-pyrimidin-2,4-dione-5-yl, 2-methoxyethyl, cyclobutyl, cyclopropylmethyl, or 3-hydroxyprop-2-yl; and
R^{3′} is hydrogen or alkyl, more preferably, hydrogen or methyl, even more preferably, or hydrogen.

Within the above preferred, more preferred and even more preferred groups, particularly preferred group of compounds is that wherein:
R^{3′} together with R³ and the nitrogen to which they are attached form heteroaryl or heterocycloamino, more preferably, 3,5-dimethylmorpholin-4-yl, 4-acetylpiperazin-1-yl, piperazinyl, morpholin-4-yl, or 3-amino-5-methylpyrazol-1-yl.

D. Yet another preferred group of compounds is that wherein:
R³ is alkyl, alkoxy, hydroxy, haloalkyl, alkylthioalkyl, cycloalkyl, cycloalkylalkyl, hydroxyalkyl, alkoxyalkyl, alkoxycarbonylalkyl, carboxyalkyl, substituted carboxyalkyl, guanidino, heterocycloamino, aminoalkyl, substituted aminoalkyl, heterocycloaminoalkyl, alkylsulfonylalkyl, alkylsulfinylalkyl, heterocycloalkyl, optionally substituted aryl, optionally substituted heteroaryl, optionally substituted aralkyl, optionally substituted heteroaralkyl, aralkenyl, aryloxyalkyl, heteroaryloxyalkyl, -[(alkylene)-O]ₘ-(alkylene)-NH₂ (where m is 1, 2, or 3), heterocycloalkylalkyl, —C(O)R¹² where R¹² is optionally substituted heteroaryl, or -(alkylene)-NR¹⁰R¹¹ where R¹⁰ and R¹¹ are independently selected from hydrogen, alkyl, optionally substituted aryl, optionally substituted heteroaryl, optionally substituted aralkyl, optionally substituted heteroaralkyl, or R¹⁰ and R¹¹ together with the nitrogen atom to which they are attached form saturated or unsaturated heterocycloamino,
preferably, 2-hydroxypyrid-6-yl, 2-chloropyrid-3-yl, 2-thio-[1,3,4]-thiadiazol-2-yl, 5,8-diphenyl-[1,2,4]triazocin-3-yl, 6-ethoxy-benzothiazol-2-yl, 6-fluoro-benzothiazol-2-yl, 3,5-dimethylisoxazol-4-yl, 5-methylisoxazol-3-ylmethyl, pyrimidin-2-yl, 3-methylpyrid-2-yl, 4-methylpyrid-2-yl, 5-methylpyrid-2-yl, 6-methylpyrid-2-yl, 4,6-dimethylpyrid-2-yl, 3-methylpyrid-4-yl, 2-methylpyrid-4-yl, 1,3-dimethylpyrazol-5-yl, 5-methylpyrazol-3-yl, 4-methylpyrimidin-2-yl, 4,6-dimethylpyrimidin-2-yl, 2,4-dimethylpyrimidin-6-yl, pyrazin-2-yl, pyrid-4-yl, pyrid-2-yl, pyrid-3-yl, pyrazol-3-yl, furan-2-ylmethyl, furan-2-ylcarbonyl, 5,6-dimethyl-[1,2,4]-triazin-3-yl, pyrimidin-4-yl, [1,3,4]-thiadiazol-2-yl, thiazol-2-yl, isoxazol-3-yl, cyclopentyl, 1H-pyrimidin-2,4-dione-5-yl, 2-methoxyethyl, cyclobutyl, cyclopropylmethyl, 3-hydroxyprop-2-yl, cyclohexylmethyl, pyrid-2-ylmethyl, pyrid-3-ylmethyl, pyrid-4-ylmethyl, pyrid-4-ylethyl, imidazol-4-ylethyl, thiophen-2-ylmethyl, cyclopentylmethyl, 2-hydroxypropyl, 3-hydroxypropyl, 2,3-dihydroxypropyl, 2-hydroxyethyl, 2-ethoxyethyl, 5-methylfuran-2-ylmethyl, cyclopropyl, cyclohexyl, 3-methoxypropyl, 1-hydroxy4-methylpent-2-yl, 1-(furan-2-yl)ethyl, 5-(dimethylaminomethyl)furan-2-ylmethyl, 5-bromofuran-2-ylmethyl, 5-chlorofuran-2-methyl, 1,3-dihydroxyprop-2-yl, 1,3-dihydroxy-2-methylprop-2-yl, 3-hydroxy-2-methylprop-2-yl, 3-methoxyprop-2-yl, 1-tert-butyl-2-hydroxyethyl, 1-hydroxy-3-methylpent-2-yl, 1,3-dihydroxy-2-hydroxymethylprop-2-yl, 1,3-dihydroxybut-2-yl, 1,2-dimethylpyrrol-5-ylmethyl, 1-methylpyrrol-2-ylmethyl, imidazol-1-ylpropyl, furan-3-ylmethyl, 2,5-dimethylfuran-3-ylmethyl, 3-(methoxycarbonyl)furan-2-ylmethyl, 6-hydroxyhexyl, N-benzylpiperidin-4-yl, N-benzylpyrrolidin-3-yl, 2-phenyloxyethyl, benzyl, morpholin-4-yl, 2-(morpholin-4-yl)ethyl, 4,5-dihydrothiazol-2-yl, piperidin-4-yl, piperidin-4-ylmethyl, 2-methylpropyl, tert-butyl, methyl, tetrahydrofuran-2-ylmethyl, hydroxy, methoxy, ethyl, propyl, 2-fluoroethyl, 2,2,2-trifluoroethyl, 2,2,3,3,3-pentafluoropropyl, 2-methylthioethyl, —(CH₂)₂O(CH₂)₂O(CH₂)₂NH₂, 1-carboxy-3-methylbut-1-yl, 2-carboxy-2-aminocarbonyl-1-carboxypropyl, 1-carboxy-2-methylbutyl, carboxymethyl, 1-carboxy-2-methylpropyl, 2-phenyl-1-carboxyethyl, 2,3-dimethoxyphenylmethyl, 3,5-dimethoxyphenylmethyl, 3,4-difluorophenylmethyl, 2,4-difluorophenylmethyl, 4-fluorophenylmethyl, 3-difluoromethoxyphenylmethyl, 2,6-dimethoxyphenylmethyl, 2-(methylsulfinyl)ethyl, 2-(methylsulfonyl)ethyl, 2-hydroxyphenyl, 4-chloro-2-hydroxyphenyl, 2-amino-4-oxo-3H-pyrimidin-6-yl, 2-cyanophenyl, 5-amino-1-carboxypentyl, 5-amino-1-aminocarbonylpentyl, 2-(4-methoxyphenyl)ethyl, or guanidino,
more preferably, 4-methylpyrimidin-2-yl, 4,6-dimethylpyrimidin-2-yl, 2,4-dimethylpyrimidin-6-yl, pyrazin-2-yl, pyridyl, pyrid-2-yl, pyrid-3-yl, pyrazol-3-yl, furan-2-ylmethyl, furan-2-ylcarbonyl, 5,6-dimethyl-[1,2,4]-triazin-3-yl, pyrimidin-4-yl, [1,3,4]-thiadiazol-2-yl, thiazol-2-yl, isoxazol-3-yl, cyclopentyl, 1H-pyrimidin-2,4-dione-5-yl, 2-methoxyethyl, cyclobutyl, cyclopropylmethyl, or 3-hydroxyprop-2-yl;
R^{3′} is hydrogen or alkyl, more preferably, hydrogen or methyl, even more preferably, hydrogen; or
R^{3′} together with R³ and the nitrogen to which they are attached form heteroaryl or heterocycloamino, more preferably, 3,5-dimethylmorpholin-4-yl, 4-acetylpiperazin-1-yl, piperazinyl, morpholin-4-yl, methyl, 2-(pyrid-4-yl)ethyl, furan-2-ylmethyl, or 3-amino-5-methylpyrazol-1-yl.

Within this group, a more preferred group of compounds is that wherein:
R¹ is hydrogen; or
R¹ is alkyl, preferably methyl, ethyl, or 2-propyl, more preferably methyl; or
R¹ is -alkylene-CONR⁸R⁹, where R⁸ and R⁹ together with the nitrogen atom to which they are attached form heterocycloamino, more preferably R¹ is 2-(piperidin-1-ylcarbonyl)ethyl, 2-(4-hydroxypiperidin-1-ylcarbonyl)ethyl, 2-(morpholin-4-ylcarbonyl)ethyl, 2-(4-acetylpiperazin-1-ylcarbonyl)ethyl, 2-(4-methylpiperidin-1-ylcarbonyl)ethyl, 2-(thiomorpholin-4-ylcarbonyl)ethyl, or 2-(4-formylpiperazin-1-ylcarbonyl)ethyl, even more preferably R¹ is 2-(4-hydroxypiperidin-1-ylcarbonyl)ethyl.

E. Yet another preferred group of compounds is that wherein:
R⁴ and R⁵ are independently of each other hydrogen, methyl, chloro, bromo, trifluoromethylthio, trifluoromethoxy, or trifluoromethyl, more preferably R⁴ and R⁵ are trifluoromethyl, even more preferably R⁴ and R⁵ are trifluoromethyl located at the 3- and 5-position of the phenyl ring.

Within this group, a more preferred group of compounds is that wherein R⁶ and R⁷ are independently of each other hydrogen, alkyl, alkoxy, hydroxy, halo, haloalkyl, amino, alkylamino, dialkylamino, or acylamino, preferably hydrogen, methyl, methoxy, or chloro, more preferably, hydrogen, 6-methyl, 7-methyl, 6-methoxy, 7-methoxy, 6-chloro, or 7-chloro, even more preferably hydrogen.

Within these preferred and more preferred groups, an even more preferred group of compounds is:
R¹ is hydrogen; or
R¹ is alkyl, preferably methyl, ethyl, or 2-propyl, more preferably methyl; or
R¹ is -alkylene-CONR⁸R⁹, where R⁸ and R⁹ together with the nitrogen atom to which they are attached form heterocycloamino, more preferably R¹ is 2-(piperidin-1-ylcarbonyl)ethyl, 2-(4-hydroxypiperidin-1-ylcarbonyl)ethyl, 2-(morpholin-4-ylcarbonyl)ethyl, 2-(4-acetylpiperazin-1-ylcarbonyl)ethyl, 2-(4-methylpiperidin-1-ylcarbonyl)ethyl, 2-(thiomorpholin-4-ylcarbonyl)ethyl, or 2-(4-formylpiperazin-1-ylcarbonyl)ethyl, even more preferably R¹ is 2-(4-hydroxypiperidin-1-ylcarbonyl)ethyl.

F. Yet another preferred group of compounds is that wherein:
R⁴ and R⁵ are trifluoromethyl and more preferably are located at the 3- and 5-position of the phenyl ring;
R⁶ and R⁷ are hydrogen;
R¹ is hydrogen, alkyl, or -alkylene-CONR⁸R⁹ where R⁸ and R⁹ together with the nitrogen atom to which they are attached form heterocycloamino, more preferably hydrogen, methyl, or 4-hydroxypiperidin-1-ylcarbonylethyl, even more preferably hydrogen or methyl;
R² is hydrogen or alkyl, more preferably hydrogen or methyl, even more preferably hydrogen; and
R^{3′} is hydrogen.

Within this group, a more preferred group is that wherein:
R³ is optionally substituted heteroaryl, more preferably, 2-hydroxypyrid-6-yl, 2-chloropyrid-3-yl, 2-thio-[1,3,4]-thiadiazol-2-yl, 5,8-diphenyl-[1,2,4]triazocin-3-yl, 6-ethoxy-benzothiazol-2-yl, 6-fluoro-benzothiazol-2-yl, 3,5-dimethylisoxazol-4-yl, 5-methylisoxazol-3-yl, pyrimidin-2-yl, 3-methylpyrid-2-yl, 4-methylpyrid-2-yl, 5-methylpyrid-2-yl, 6-methylpyrid-2-yl, 4,6-dimethylpyrid-2-yl, 3-methylpyrid-4-yl, 2-methylpyrid-4-yl, 1,3-dimethylpyrazol-5-yl, 5-methylpyrazol-3-yl, 4-methylpyrimidin-2-yl, 4,6-dimethylpyrimidin-2-yl, 2,4-dimethylpyrimidin-6-yl, pyrazin-2-yl, pyrid-4-yl, pyrid-2-yl, pyrid-3-yl, pyrazol-3-yl, 5,6-dimethyl-[1,2,4]-triazin-3-yl, pyrimidin-4-yl, [1,3,4]-thiadiazol-2-yl, thiazol-2-yl, or isoxazol-3-yl,
even more preferably, 4-methylpyrimidin-2-yl, 4,6-dimethylpyrimidin-2-yl, 2,4-dimethylpyrimidin-6-yl, pyrazin-2-yl, pyrid-4-yl, pyrid-2-yl, pyrid-3-yl, pyrazol-3-yl, 5,6-dimethyl-[1,2,4]-triazin-3-yl, pyrimidin-4-yl, [1,3,4]-thiadiazol-2-yl, thiazol-2-yl, or isoxazol-3-yl.

Within this group, a more preferred group is that wherein:
R³ is optionally substituted heteroaralkyl, hydroxyalkyl, cycloalkyl, cycloalkylalkyl, alkoxyalkyl, —C(O)R¹² where R¹² is optionally substituted heteroaryl, or heterocycloalkyl, more preferably, furan-2-ylcarbonyl, furan-2-ylmethyl, cyclohexylmethyl, pyrid-2-ylmethyl, pyrid-3-ylmethyl, pyrid-4-ylmethyl, pyrid-4-ylethyl, imidazol-4-ylethyl, thiophen-2-ylmethyl, cyclopentylmethyl, 2-hydroxypropyl, 3-hydroxyprop-2-yl, hydroxypropyl, 2,3-dihydroxypropyl, hydroxyethyl, ethoxyethyl, 5-methylfuran-2-ylmethyl, cyclopropyl, cyclohexyl, 3-methoxypropyl, 1-hydroxy4-methylpent-2-yl, 1-(furan-2-yl)ethyl, 5-(dimethylaminomethyl)furan-2-ylmethyl, 5-bromofuran-2-ylmethyl, 5-chlorofuran-2-methyl, 1,3-dihydroxyprop-2-yl, 1,3-dihydroxy-2-methylprop-2-yl, 3-hydroxy-2-methylprop-2-yl, 3-methoxyprop-2-yl, 1-tert-butyl-2-hydroxyethyl, 1-hydroxy-3-methylpent-2-yl, 1,3-dihydroxy-2-hydroxymethylprop-2-yl, 1,3-dihydroxybut-2-yl, 1,2-dimethylpyrrol-5-ylmethyl, 1-methylpyrrol-2-ylmethyl, imidazol-1-ylpropyl, furan-3-ylmethyl, 2,5-dimethylfuran-3-ylmethyl, 3-methoxycarbonylfuran-2-ylmethyl, 6-hydroxyhexyl, cyclopentyl, 1H-pyrimidin-2,4-dione-5-yl, 2-methoxyethyl, cyclobutyl, cyclopropylmethyl, furan-2-ylmethyl, or 3-hydroxyprop-2-yl,
even more preferably, cyclopentyl, 1H-pyrimidin-2,4-dione-5-yl, 2-methoxyethyl, cyclobutyl, cyclopropylmethyl, furan-2-ylmethyl, furan-2-ylcarbonyl, or 3-hydroxyprop-2-yl.

G. Yet another preferred group of compounds is that wherein the stereochemistry at *C and **C, as indicated in the following structure, is trans i.e., (R,S) or (S,R).

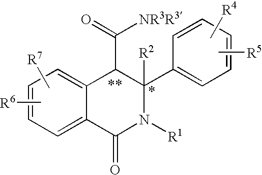

I. Representative compounds of Formula I, where R^{3′}, R⁶ and R⁷ are hydrogen and where R⁴ and R⁵ are trifluoromethyl, are disclosed in Table I. The stereochemistry at *C and **C of compounds prepared using methods described herein may be determined using standard analytical techniques known to one of ordinary skill in the art.

TABLE I

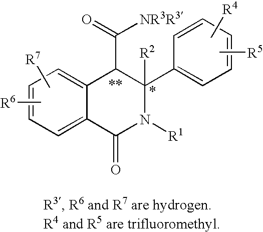

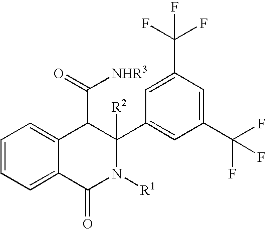

| Cmpd | | | |
|---|---|---|---|
| No. | R¹ | R² | R³ |
| 1 | CH₃ | H | furan-2-ylmethyl |
| 2 | CH₃ | CH₃ | furan-2-ylmethyl |
| 3 | H | H | furan-2-ylmethyl |
| 4 | 2-(piperidin-1-ylcarbonyl)ethyl | H | 2-methoxyethyl |
| 5 | CH₃ | H | 2-methoxyethyl |
| 6 | H | H | cyclopentyl |
| 7 | CH₃ | H | 1H-pyrimidin-2,4-dione-5-yl |
| 8 | CH₃ | H | [1,3,4]-thiadiazol-2-yl |
| 9 | H | H | 2-methoxyethyl |
| 10 | CH₃ | H | 3-hydroxyprop-2-yl |
| 11 | CH₃ | H | 1-hydroxy-3,3-dimethyl-2-butyl |
| 12 | 2-(4-hydroxypiperidin-1-yl- | H | furan-2-ylmethyl |
| | carbonyl)ethyl | | |
| 13 | CH₃ | H | cyclopropylmethyl |
| 14 | CH₃ | CH₃ | thiophen-2-ylmethyl |
| 15 | CH₃ | H | 2-ethoxyethyl |
| 16 | CH₃ | H | 2-fluoroethyl |
| 17 | CH₃ | CH₃ | 2-methoxyethyl |
| 18 | CH₃ | H | 2-methylthioethyl |
| 19 | CH₃ | H | 3-hydroxypropyl |
| 20 | CH₃ | H | cyclobutyl |
| 21 | CH₃ | H | n-propyl |
| 22 | CH₃ | H | 1-furan-2-ylethyl |
| 23 | CH₃ | H | S-methylfuran-2-yl-methyl |
| 24 | CH₃ | CH₃ | cyclopropylmethyl |
| 25 | CH₃ | H | 2-hydroxyethyl |
| 26 | CH₃ | H | cyclopropyl |
| 27 | CH₃ | H | tetrahydrofuran-2-ylmethyl |
| 28 | H | H | 5-methylfuran-2-yl-methyl |
| 29 | CH₃ | H | thiazol-2-yl |
| 30 | CH₃ | H | pyridin-2-ylmethyl |
| 31 | 2-piperidin-1-ylcarbonylethyl | H | furan-2-yl-methyl |
| 32 | CH₃ | H | cyclopentyl |
| 33 | H | H | cyclopropylmethyl |
| 34 | cyclopropyl | H | furan-2-ylmethyl |
| 35 | H | H | thiazol-2-yl |
| 36 | 2-(4-hydroxypiperidin-1-yl- | H | cyclopentyl |
| | carbonyl)ethyl | | |
| 37 | CH₃ | H | benzyl |
| 38 | 2-dimethylaminocarbonylethyl | H | benzyl |
| 39 | 2-dimethylaminocarbonylethyl | H | cyclopentyl |
| 40 | CH₃ | H | furan-3-ylmethyl |
| 41 | CH₃ | H | pyridin-3-ylmethyl |
| 42 | cyclopropyl | H | cyclopentyl |
| 43 | cyclopropyl | H | 2-methoxyethyl |
| 44 | CH₃ | H | 2-chloro-pyridin-3-yI |
| 45 | H | H | 4,5-dihydrothiazol-2-yl |
| 46 | 2-(4-hydroxypiperidin-1- | H | cyclopropylmethyl |
| | ylcarbonyl)-ethyl | | |
| 47 | CH₃ | H | 4,5-dihydrothiazol-2-yl |
| 48 | 2-dimethylaminocarbonylethyl | H | 2-methoxyethyl |
| 49 | 2-(morpholin-4-ylcarbonyl)- | H | benzyl |
| | ethyl | | |
| 50 | 2-(4-hydroxypiperidin-1- | H | benzyl |
| | ylcarbonyl)-ethyl | | |
| 51 | 2-(4-acetylpiperazin-1-yl- | H | cyclopentyl |
| | carbonyl)ethyl | | |
| 52 | 2-morpholin-4-ylethyl | H | cyclopentyl |
| 53 | 13 (CH₂)₂C(O){N(CH₃)— | H | benzyl |
| | [(CH₂)₂OCH₃]} | | |
| 54 | 2-propyl | H | furan-2-ylmethyl |
| 55 | 2-dimethylaminocarbonylethyl | H | cyclopropylmethyl |
| 56 | 2-dimethylaminocarbonylethyl | H | furan-2-ylmethyl |
| 57 | H | H | benzyl |
| 58 | 2-piperidin-1-ylcarbonylethyl | H | cyclopentyl |
| 59 | 2-propyl | H | cyclopentyl |
| 60 | CH₃ | H | pyridin-4-ylmethyl |
| 61 | 2-methoxyethyl | H | cyclopentyl |
| 62 | CH₃ | H | cyclopentylmethyl |
| 63 | —(CH₂)₂CO{N(CH₃)— | H | benzyl |
| | [(CH₂)₃CH₃]} | | |
| 64 | 2-methoxyethyl | H | 2-methoxyethyl |
| 65 | morpholin-4-ylethyl | H | furan-2-ylmethyl |
| 66 | —(CH₂)₂CO{N(CH₃)(benzyl)} | H | benzyl |
| 67 | —(CH₂)₂CO{N(CH₃)[2-3,4- | H | benzyl |
| | dimethyoxyphenyl)ethyl]} | | |
| 68 | CH₃ | H | 2-imidazol-4-ylethyl |
| 69 | 2-(4-acetylpiperazin-1-yl- | H | cyclopropylmethyl |
| | carbonyl)ethyl | | |
| 70 | furan-2-ylmethyl | H | 2-methoxyethyl |
| 71 | 2-(4-acetylpiperazin-1-yl- | H | 2-methoxyethyl |
| | carbonyl)ethyl | | |
| 72 | H | H | furan-3-ylmethyl |
| 73 | H | H | morpholin-4-yl |
| 74 | 2-morpholin-4-ylethyl | H | 2-methoxyethyl |
| 75 | CH₃ | H | 2-hydroxypyridin-6-yl |
| 76 | CH₃ | H | 2-pyridin-4-ylethyl |
| 77 | 2-(4-formylpiperazin-1-yl- | H | benzyl |
| | carbonyl)ethyl | | |
| 78 | H | H | 2-morpholin-4-ylethyl |
| 79 | 2-propyl | H | 2-methoxyethyl |
| 80 | furan-2-ylmethyl | H | cyclopentyl |
| 81 | CH₃ | H | piperidin-4-ylmethyl |
| 82 | CH₃ | H | 5-dimethylaminofuran-2- |
| | | | ylmethyl |
| 83 | CH₃ | H | 5-bromofuran-2-yl-methyl |
| 84 | CH₃ | H | hydroxy |
| 85 | CH₃ | H | isoxazol-3-yl |
| 86 | CH₃ | H | 1-carboxy-2-methyl-1-butyl |
| 87 | H | H | isoxazol-3-yl |
| 88 | CH₃ | H | 2,4-dimethylpyrid-6-yl |
| 89 | CH₃ | H | pyrazol-3-yl |
| 90 | CH₃ | H | 4-methylpyrimidin-2-yl |
| 91 | CH₃ | H | 4,6-dimethylpyrimidin-2-yl |
| 92 | CH₃ | H | 2,4-dimethylpyrimidin-6-yl |
| 93 | CH₃ | H | pyrazin-2-yl |
| 94 | CH₃ | H | pyridin-4-yl |
| 95 | CH₃ | H | pyridin-2-yl |
| 96 | CH₃ | H | pyridin-3-yI |
| 97 | CH₃ | H | 5,6-dimethyl-[1,2,4]triazin-3-yl |
| 98 | H | H | 5,6-dimethyl-[1,2,4]triazin-3-yl |
| 99 | CH₃ | H | pyrimidin-4-yl |
| 100 | CH₃ | H | fuxan-2-ylcarbonyl |
| 101 | CH₃ | H | 2-methylpyrid-4-yl |
| 102 | CH₃ | H | 2-methylpyrid-6-yl |
| 103 | CH₃ | H | 3-methylpyrid-4-yl |
| 104 | CH₃ | H | 3-methylpyrid-6-yl |
| 105 | CH₃ | H | 4-methylpyrid-2-yl |
| 106 | CH₃ | H | pyrimidin-2-yl |
| 107 | ethoxycarbonylethyl | H | 2-(4-methoxyphenyl)ethyl |
| 108 | aminocarbonylethyl | H | N-benzylpiperidin-4-yl |
| 109 | aminocarbonylethyl | H | N-benzylpyrrolidin-3-yl |
| 110 | aminocarbonylethyl | H | 2-phenoxyethyl |
| 111 | [2-(3- | H | benzyl |
| | methoxyphenyl)ethyl]amino- | | |
| | carbonylethyl | | |
| 112 | tetrahydrofuran-2-ylmethyl | H | benzyl |
| 113 | (4-methylpiperidin-1- | H | benzyl |
| | yl)carbonylethyl | | |
| 114 | thiomorpholin-4- | H | benzyl |
| | ylcarbonylethyl | | |
| 115 | 2-(3- | H | 2-methoxyethyl |
| | methoxyphenyl)ethylamino- | | |
| | carbonylethyl | | |
| 116 | 4-acetylpiperazin-1- | H | furan-2-ylmethyl |
| | ylcarbonylethyl | | |
| 117 | 2-methoxyethyl | H | fuxan-2-ylmethyl |
| 118 | H | H | cyclohexylmethyl |
| 119 | 4-methoxyphenylmethyl | H | 2-methoxyethyl |
| 120 | 4-methoxyphenylmethyl | H | cyclopropylmethyl |
| 121 | CH₃ | H | 2,3-dihydroxypropyl |
| 122 | CH₃ | H | methoxy |
| 123 | methoxycarbonylpentyl | H | cyclopentyl |
| 124 | CH₃ | H | 2-aminoethyloxyethyloxyethyl |
| 125 | CH₃ | H | 2-carboxy-3-methylbutyl |
| 126 | CH₃ | H | 2-hydroxy-1,1- |
| | | | (dihydroxyniethyl)ethyl |
| 127 | CH₃ | H | 3-aminocarbonyl-1- |
| | | | carboxypropyl |
| 128 | CH₃ | H | 1-carboxy-2-phenylethyl |
| 129 | CH₃ | H | N-methylpyrrol-2-ylmethyl |
| 130 | H | H | 1,2-dimethylpyrrol-5-ylmethyl |
| 131 | CH₃ | H | 2,3-dimethoxyphenylmethyl |
| 132 | CH₃ | H | 2,4-difluorophenylmethyl |
| 133 | CH₃ | H | 3- |
| | | | (difluoromethoxy)phenylmethyl |
| 134 | CH₃ | H | imidazol-1-ylpropyl |
| 135 | CH₃ | H | 2-(methylsulfinyl)ethyl |
| 136 | CH₃ | H | 2-(methylsulfonyl)ethyl |
| 137 | CH₃ | H | 2-amino-4-oxo-3H-pyrimidin-6-yl |
| 138 | H | H | 4-chloro-2-hydroxyphenyl |
| 139 | H | H | 2-cyanophenyl |
| 140 | CH₃ | H | 3-methoxycarbonylfuran-2- |
| | | | ylmethyl |
| 141 | H | H | 5-niercapto-[1,3,4]-thiadiazol-2- |
| | | | yl |
| 142 | CH₃ | H | 5-amino-1-carboxypentyl |
| 143 | CH₃ | H | 5-amino-1-aminocarbonylpentyl |
| 144 | CH₃ | H | 5,8-diphenyl-[1,2,4]triazocin-3-yl |
| 145 | CH₃ | H | 6-ethoxybenzothiazol-2-yl |
| 146 | CH₃ | H | guanidino |
| 147 | CH₃ | H | 3,5-dimethylisoxazol-4-yl |
| 148 | CH₃ | H | piperidin-4-yl |
| 149 | CH₃ | H | 1,3-pyrazol-5-yl |

are named as
3,5-bis-trifluoromethylphenyl)-4-[(furan-2-ylmethyl)-aminocarbonyl]-2-methyl-1-oxo-1,2,3,4-tetrahydroisoquinoline;
3,5-bis-trifluoromethylphenyl)-4-[(furan-2-ylmethyl)-aminocarbonyl]-2,3-dimethyl-1-oxo-1,2,3,4-tetrahydroisoquinoline;
3,5-bis-trifluoromethylphenyl)-4-[(furan-2-ylmethyl)-aminocarbonyl]-1-oxo-1,2,3,4-tetrahydroisoquinoline;
3,5-bis-trifluoromethylphenyl)-4-[(2-methoxyethyl)-aminocarbonyl]-2-[2-(piperidin-1-ylcarbonyl)ethyl]-1-oxo-1,2,3,4-tetrahydroisoquinoline;
3-(3,5-bis-trifluoromethylphenyl)-4-[(2-methoxyethyl)-aminocarbonyl]-2-methyl-1-oxo-1,2,3,4-tetrahydroisoquinoline;
3-(3,5-bis-trifluoromethylphenyl)-4-[(cyclopentyl)-aminocarbonyl]-1-oxo-1,2,3,4-tetrahydroisoquinoline;
3-(3,5-bis-trifluoromethylphenyl)-4-[(1H-pyrimidin-2,4-dione-5-yl)-aminocarbonyl]-2-methyl-1-oxo-1,2,3,4-tetrahydroisoquinoline;
3-(3,5-bis-trifluoromethylphenyl)-4-{([1,3,4]-thiadiazol-2-yl)-aminocarbonyl}-2-methyl-1-oxo-1,2,3,4-tetrahydroisoquinoline;
3-(3,5-bis-trifluoromethylphenyl)-4-[(2-methoxyethyl)-aminocarbonyl]-1-oxo-1,2,3,4-tetrahydroisoquinoline;
3-(3,5-bis-trifluoromethylphenyl)-4-[(3-hydroxyprop-2-yl)-aminocarbonyl]-2-methyl-1-oxo-1,2,3,4-tetrahydroisoquinoline;
3-(3,5-bis-trifluoromethylphenyl)-4-[(1-hydroxy-3,3-dimethyl-2-butyl)-aminocarbonyl]-2-methyl-1-oxo-1,2,3,4-tetrahydroisoquinoline;
3-(3,5-bis-trifluoromethylphenyl)-4-[(furan-2-ylmethyl)-aminocarbonyl]-2-[2-(4-hydroxypiperidin-1-yl-carbonyl)ethyl]-1-oxo-1,2,3,4-tetrahydroisoquinoline;
3-(3,5-bis-trifluoromethylphenyl)-4-[(cyclopropylmethyl)-aminocarbonyl]-2-methyl-1-oxo-1,2,3,4-tetrahydroisoquinoline;
3-(3,5-bis-trifluoromethylphenyl)-4-[(thiophen-2-ylmethyl)-aminocarbonyl]-2,3-dimethyl-1-oxo-1,2,3,4-tetrahydroisoquinoline;
3-(3,5-bis-trifluoromethylphenyl)-4-[(2-ethoxyethyl)-aminocarbonyl]-2-methyl-1-oxo-1,2,3,4-tetrahydroisoquinoline;
3-(3,5-bis-trifluoromethylphenyl)-4-[(2-fluoroethyl)-aminocarbonyl]-2-methyl-1-oxo-1,2,3,4-tetrahydroisoquinoline;
3-(3,5-bis-trifluoromethylphenyl)-4-[(2-methoxyethyl)-aminocarbonyl]-2,3-dimethyl-1-oxo-1,2,3,4-tetrahydroisoquinoline;
3-(3,5-bis-trifluoromethylphenyl)-4-[(2-methylthioethyl)-aminocarbonyl]-2-methyl-1-oxo-1,2,3,4-tetrahydroisoquinoline;
3-(3,5-bis-trifluoromethylphenyl)-4-[(3-hydroxypropyl)-aminocarbonyl]-2-methyl-1-oxo-1,2,3,4-tetrahydroisoquinoline;
3-(3,5-bis-trifluoromethylphenyl)-4-[(cyclobutyl)-aminocarbonyl]-2-methyl-1-oxo-1,2,3,4-tetrahydroisoquinoline;
3-(3,5-bis-trifluoromethylphenyl)-4-[(n-propyl)-aminocarbonyl]-2-methyl-1-oxo-1,2,3,4-tetrahydroisoquinoline;
3-(3,5-bis-trifluoromethylphenyl)-4-[(1-furan-2-ylethyl)-aminocarbonyl]-2-methyl-1-oxo-1,2,3,4-tetrahydroisoquinoline;
3-(3,5-bis-trifluoromethylphenyl)-4-[(5-methylfuran-2-yl-methyl)-aminocarbonyl]-2-methyl-1-oxo-1,2,3,4-tetrahydroisoquinoline;
3-(3,5-bis-trifluoromethylphenyl)-4-[(cyclopropylmethyl)-aminocarbonyl]-2,3-dimethyl-1-oxo-1,2,3,4-tetrahydroisoquinoline;
3-(3,5-bis-trifluoromethylphenyl)-4-[(2-hydroxyethyl)-aminocarbonyl]-2-methyl-1-oxo-1,2,3,4-tetrahydroisoquinoline;
3-(3,5-bis-trifluoromethylphenyl)-4-[(cyclopropyl)-aminocarbonyl]-2-methyl-1-oxo-1,2,3,4-tetrahydroisoquinoline;
3-(3,5-bis-trifluoromethylphenyl)-4-[(tetrahydrofuran-2-ylmethyl)-aminocarbonyl]-2-methyl-1-oxo-1,2,3,4-tetrahydroisoquinoline;
3-(3,5-bis-trifluoromethylphenyl)-4-[(5-methylfuran-2-yl-methyl)-aminocarbonyl]-1-oxo-1,2,3,4-tetrahydroisoquinoline;
3-(3,5-bis-trifluoromethylphenyl)-4-[(thiazol-2-yl)-aminocarbonyl-2-methyl-1-oxo-1,2,3,4-tetrahydroisoquinoline;
3-(3,5-bis-trifluoromethylphenyl)-4-[(pyridin-2-ylmethyl)-aminocarbonyl]-2-methyl-1-oxo-1,2,3,4-tetrahydroisoquinoline;
3-(3,5-bis-trifluoromethylphenyl)-4-[(furan-2-yl-methyl)-aminocarbonyl]-2-(2-piperidin-1-ylcarbonylethyl)-1-oxo-1,2,3,4-tetrahydroisoquinoline;
3-(3,5-bis-trifluoromethylphenyl)-4-[(cyclopentyl)-aminocarbonyl]-2-methyl-1-oxo-1,2,3,4-tetrahydroisoquinoline;
3-(3,5-bis-trifluoromethylphenyl)-4-[(cyclopropylmethyl)-aminocarbonyl]-1-oxo-1,2,3,4-tetrahydroisoquinoline;
3-(3,5-bis-trifluoromethylphenyl)-4-[(furan-2-ylmethyl)-aminocarbonyl]-2-(cyclopropyl)-1-oxo-1,2,3,4-tetrahydroisoquinoline;
3-(3,5-bis-trifluoromethylphenyl)-4-[(thiazol-2-yl)-aminocarbonyl]-1-oxo-1,2,3,4-tetrahydroisoquinoline;
3-(3,5-bis-trifluoromethylphenyl)-4-[(cyclopentyl)-aminocarbonyl]-2-[2-(4-hydroxypiperidin-1-yl-carbonyl)ethyl]-1-oxo-1,2,3,4-tetrahydroisoquinoline;
3-(3,5-bis-trifluoromethylphenyl)-4-[(benzyl)-aminocarbonyl]-2-methyl-1-oxo-1,2,3,4-tetrahydroisoquinoline;
3-(3,5-bis-trifluoromethylphenyl)-4-[(benzyl)-aminocarbonyl]-2-(2-dimethylaminocarbonylethyl)-1-oxo-1,2,3,4-tetrahydroisoquinoline;
3-(3,5-bis-trifluoromethylphenyl)-4-[(cyclopentyl)-aminocarbonyl]-2-(2-dimethylaminocarbonylethyl)-1-oxo-1,2,3,4-tetrahydroisoquinoline;
3-(3,5-bis-trifluoromethylphenyl)-4-[(furan-3-ylmethyl)-aminocarbonyl]-2-methyl-1-oxo-1,2,3,4-tetrahydroisoquinoline; 3-(3,5-bis-trifluoromethylphenyl)-4-[(pyridin-3-ylmethyl)-aminocarbonyl]-2-methyl-1-oxo-1,2,3,4-tetrahydroisoquinoline;
3-(3,5-bis-trifluoromethylphenyl)-4-[(cyclopentyl)-aminocarbonyl]-2-cyclopropyl-1-oxo-1,2,3,4-tetrahydroisoquinoline;
3-(3,5-bis-trifluoromethylphenyl)-4-[(2-methoxyethyl)-aminocarbonyl]-2-cyclopropyl-1-oxo-1,2,3,4-tetrahydroisoquinoline;
3-(3,5-bis-trifluoromethylphenyl)-4-[(2-chloro-pyridin-3-yl)-aminocarbonyl]-2-methyl-1-oxo-1,2,3,4-tetrahydroisoquinoline;
3-(3,5-bis-trifluoromethylphenyl)-4-[(4,5-dihydrothiazol-2-yl)-aminocarbonyl]-1-oxo-1,2,3,4-tetrahydroisoquinoline; 3-(3,5-bis-trifluoromethylphenyl)-4-[(cyclopropylmethyl)-aminocarbonyl]-2-[2-(4-hydroxypiperidin-1-ylcarbonyl)-ethyl]-1-oxo-1,2,3,4-tetrahydroisoquinoline;
3-(3,5-bis-trifluoromethylphenyl)-4-[(4,5-dihydrothiazol-2-yl)-aminocarbonyl]-2-methyl-1-oxo-1,2,3,4-tetrahydroisoquinoline;
3-(3,5-bis-trifluoromethylphenyl)-4-[(2-methoxyethyl)-aminocarbonyl]-2-(2-dimethylaminocarbonylethyl)-1-oxo-1,2,3,4-tetrahydroisoquinoline;
3-(3,5-bis-trifluoromethylphenyl)-4-[(benzyl)-aminocarbonyl]-2-[2-(morpholin-4-ylcarbonyl)-ethyl]-1-oxo-1,2,3,4-tetrahydroisoquinoline;
3-(3,5-bis-trifluoromethylphenyl)-4-[(benzyl)-aminocarbonyl]-2-[2-(4-hydroxypiperidin-1-yl-carbonyl)-ethyl]-1-oxo-1,2,3,4-tetrahydroisoquinoline; 3-(3,5-bis-trifluoromethylphenyl)-4-[(cyclopentyl)-aminocarbonyl]-2-[2-(4-acetylpiperazin-1-yl-carbonyl)ethyl]-1-oxo-1,2,3,4-tetrahydroisoquinoline;
3-(3,5-bis-trifluoromethylphenyl)-4-[(cyclopentyl)-aminocarbonyl]-2-(2-morpholin-4-ylethyl)-1-oxo-1,2,3,4-tetrahydroisoquinoline;
3-(3,5-bis-trifluoromethylphenyl)-4-[(benzyl)-aminocarbonyl]-2-{(CH₂)₂C(O){N(CH₃)—[(CH₂)₂OCH₃]}}-1-oxo-1,2,3,4-tetrahydroisoquinoline;
3-(3,5-bis-trifluoromethylphenyl)-4-[(furan-2-ylmethyl)-aminocarbonyl]-2-(2-propyl)-1-oxo-1,2,3,4-tetrahydroisoquinoline;
b 3-(3,5-bis-trifluoromethylphenyl)-4-[(cyclopropylmethyl)-aminocarbonyl]-2-(2-dimethylaminocarbonylethyl)-1-oxo-1,2,3,4-tetrahydroisoquinoline;
3-(3,5-bis-trifluoromethylphenyl)-4-[(furan-2-ylmethyl)-aminocarbonyl]-2-(2-dimethylaminocarbonylethyl)-1-oxo-1,2,3,4-tetrahydroisoquinoline;
3-(3,5-bis-trifluoromethylphenyl)-4-[(benzyl)-aminocarbonyl]-1-oxo-1,2,3,4-tetrahydroisoquinoline;
3-(3,5-bis-trifluoromethylphenyl)-4-[(cyclopentyl)-aminocarbonyl]-2-(2-piperidin-1-ylcarbonylethyl)-1-oxo-1,2,3,4-tetrahydroisoquinoline;
3-(3,5-bis-trifluoromethylphenyl)-4-[(cyclopentyl)-aminocarbonyl]-2-(2-propyl)-1-oxo-1,2,3,4-tetrahydroisoquinoline;
3-(3,5-bis-trifluoromethylphenyl)-4-[(pyridin-4-ylmethyl)-aminocarbonyl]-2-methyl-1-oxo-1,2,3,4-tetrahydroisoquinoline;
3-(3,5-bis-trifluoromethylphenyl)-4-[(cyclopentyl)-aminocarbonyl]-2-(2-methoxyethyl)-1-oxo-1,2,3,4-tetrahydroisoquinoline;
3-(3,5-bis-trifluoromethylphenyl)-4-[(cyclopentylmethyl)-aminocarbonyl]-2-methyl-1-oxo-1,2,3,4-tetrahydroisoquinoline;
3-(3,5-bis-trifluoromethylphenyl)-4-[(benzyl)-aminocarbonyl]-2-{(CH₂)₂C(O){N(CH₃)—[(CH₂)₃CH₃]}}-1-oxo-1,2,3,4-tetrahydroisoquinoline;
3-(3,5-bis-trifluoromethylphenyl)-4-[(2-methoxyethyl)-aminocarbonyl]-2-(2-methoxyethyl)-1-oxo-1,2,3,4-tetrahydroisoquinoline;
3-(3,5-bis-trifluoromethylphenyl)-4-[(furan-2-ylmethyl)-aminocarbonyl]-2-(morpholin-4-ylethyl)-1-oxo-1,2,3,4-tetrahydroisoquinoline;
3-(3,5-bis-trifluoromethylphenyl)-4-[(benzyl)-aminocarbonyl]-2-{(CH₂)₂CO{N(CH₃)(benzyl)}}-1-oxo-1,2,3,4-tetrahydroisoquinoline;
3-(3,5-bis-trifluoromethylphenyl)-4-[(benzyl)-aminocarbonyl]-2-{(CH₂)₂CO{N(CH₃)[2-(3,4-dimethyoxyphenyl)ethyl]}}-1-oxo-1,2,3,4-tetrahydroisoquinoline;
3-(3,5-bis-trifluoromethylphenyl)-4-[(2-imidazol-4-ylethyl)-aminocarbonyl]-2-methyl-1-oxo-1,2,3,4-tetrahydroisoquinoline;
3-(3,5-bis-trifluoromethylphenyl)-4-[(cyclopropylmethyl)-aminocarbonyl]-2-[2-(4-acetylpiperazin-1-yl-carbonyl)ethyl]-1-oxo-1,2,3,4-tetrahydroisoquinoline;
3-(3,5-bis-trifluoromethylphenyl)-4-[(2-methoxyethyl)-aminocarbonyl]-2-(furan-2-ylmethyl)-1-oxo-1,2,3,4-tetrahydroisoquinoline;
3-(3,5-bis-trifluoromethylphenyl)-4-[(2-methoxyethyl)-aminocarbonyl]-2-[2-(4-acetylpiperazin-1-yl-carbonyl)ethyl]-1-oxo-1,2,3,4-tetrahydroisoquinoline;
3-(3,5-bis-trifluoromethylphenyl)-4-[(furan-3-ylmethyl)-aminocarbonyl]-1-oxo-1,2,3,4-tetrahydroisoquinoline;
3-(3,5-bis-trifluoromethylphenyl)-4-[(morpholin-4-yl)-aminocarbonyl]-1-oxo-1,2,3,4-tetrahydroisoquinoline;
3-(3,5-bis-trifluoromethylphenyl)-4-[(2-methoxyethyl)-aminocarbonyl]-2-(2-morpholin-4-ylethyl)-1-oxo-1,2,3,4-tetrahydroisoquinoline;
3-(3,5-bis-trifluoromethylphenyl)-4-[(2-hydroxypyridin-6-yl)-aminocarbonyl]-2-methyl-1-oxo-1,2,3,4-tetrahydroisoquinoline;
3-(3,5-bis-trifluoromethylphenyl)-4-[(2-pyridin-4-ylethyl)-aminocarbonyl]-2-methyl-1-oxo-1,2,3,4-tetrahydroisoquinoline;
3-(3,5-bis-trifluoromethylphenyl)-4-[(benzyl)-aminocarbonyl]-2-[2-(4-formylpiperazin-1-yl-carbonyl)ethyl]-1-oxo-1,2,3,4-tetrahydroisoquinoline;
3-(3,5-bis-trifluoromethylphenyl)-4-[(2-morpholin-4-ylethyl)-aminocarbonyl]-1-oxo-1,2,3,4-tetrahydroisoquinoline;
3-(3,5-bis-trifluoromethylphenyl)-4-[(2-methoxyethyl)-aminocarbonyl]-2-(2-propyl)-1-oxo-1,2,3,4-tetrahydroisoquinoline;
3-(3,5-bis-trifluoromethylphenyl)-4-[(cyclopentyl)-aminocarbonyl]-2-(furan-2-ylmethyl)-1-oxo-1,2,3,4-tetrahydroisoquinoline;
3-(3,5-bis-trifluoromethylphenyl)4-[(piperidin-4-ylmethyl)-aminocarbonyl]-2-methyl-1-oxo-1,2,3,4-tetrahydroisoquinoline;
3-(3,5-bis-trifluoromethylphenyl)-4-[(5-dimethylaminofuran-2-ylmethyl)-aminocarbonyl]-2-methyl-1-oxo-1,2,3,4-tetrahydroisoquinoline;
3-(3,5-bis-trifluoromethylphenyl)-4-[(5-bromofuran-2-yl-methyl)-aminocarbonyl]-2-methyl-1-oxo-1,2,3,4-tetrahydroisoquinoline;
3-(3,5-bis-trifluoromethylphenyl)-4-[(hydroxy)-aminocarbonyl]-2-methyl-1-oxo-1,2,3,4-tetrahydroisoquinoline;
3-(3,5-bis-trifluoromethylphenyl)-4-[(isoxazol-3-yl)-aminocarbonyl]-2-methyl-1-oxo-1,2,3,4-tetrahydroisoquinoline;
3-(3,5-bis-trifluoromethylphenyl)-4-[(1-carboxy-2-methyl-1-butyl)-aminocarbonyl]-2-methyl-1-oxo-1,2,3,4-tetrahydroisoquinoline;
3-(3,5-bis-trifluoromethylphenyl)-4-[(isoxazol-3-yl)-aminocarbonyl]-1-oxo-1,2,3,4-tetrahydroisoquinoline;
3-(3,5-bis-trifluoromethylphenyl)-4-[(2,4-dimethylpyrid-6-yl)-aminocarbonyl]-2-methyl-1-oxo-1,2,3,4-tetrahydroisoquinoline;
3-(3,5-bis-trifluoromethylphenyl)-4-[(pyrazol-3-yl)-aminocarbonyl]-2-methyl-1-oxo-1,2,3,4-tetrahydroisoquinoline;
3-(3,5-bis-trifluoromethylphenyl)-4-[(4-methylpyrimidin-2-yl)-aminocarbonyl]-2-methyl-1-oxo-1,2,3,4-tetrahydroisoquinoline;
3-(3,5-bis-trifluoromethylphenyl)-4-[(4,6-dimethylpyrimidin-2-yl)-aminocarbonyl]-2-methyl-1-oxo-1,2,3,4-tetrahydroisoquinoline;
3-(3,5-bis-trifluoromethylphenyl)-4-[(2,4-dimethylpyrimidin-6-yl)-aminocarbonyl]-2-methyl-1-oxo-1,2,3,4-tetrahydroisoquinoline;
3-(3,5-bis-trifluoromethylphenyl)-4-[(pyrazin-2-yl)-aminocarbonyl]-2-methyl-1-oxo-1,2,3,4-tetrahydroisoquinoline;
3-(3,5-bis-trifluoromethylphenyl)-4-[(pyridin-4-yl)-aminocarbonyl]-2-methyl-1-oxo-1,2,3,4-tetrahydroisoquinoline;
3-(3,5-bis-trifluoromethylphenyl)-4-[(pyridin-2-yl)-aminocarbonyl]-2-methyl-1-oxo-1,2,3,4-tetrahydroisoquinoline;
3-(3,5-bis-trifluoromethylphenyl)-4-[(pyridin-3-yl)-aminocarbonyl]-2-methyl-1-oxo-1,2,3,4-tetrahydroisoquinoline;
3-(3,5-bis-trifluoromethylphenyl)-4-[(5,6-dimethyl-[1,2,4]triazin-3-yl)-aminocarbonyl]-2-methyl-1-oxo-1,2,3,4-tetrahydroisoquinoline;
3-(3,5-bis-trifluoromethylphenyl)-4-[(5,6-dimethyl-[1,2,4]triazin-3-yl)-aminocarbonyl]-1-oxo-1,2,3,4-tetrahydroisoquinoline;
3-(3,5-bis-trifluoromethylphenyl)-4-[(pyrimidin-4-yl)-aminocarbonyl]-2-methyl-1-oxo-1,2,3,4-tetrahydroisoquinoline;
3-(3,5-bis-trifluoromethylphenyl)-4-[(furan-2-ylcarbonyl)-aminocarbonyl]-2-methyl-1-oxo-1,2,3,4-tetrahydroisoquinoline;
3-(3,5-bis-trifluoromethylphenyl)-4-[(2-methylpyrid-4-yl)-aminocarbonyl]-2-methyl-1-oxo-1,2,3,4-tetrahydroisoquinoline;
3-(3,5-bis-trifluoromethylphenyl)-4-[(2-methylpyrid-6-yl)-aminocarbonyl]-2-methyl-1-oxo-1,2,3,4-tetrahydroisoquinoline;
3-(3,5-bis-trifluoromethylphenyl)-4-[(3-methylpyrid-4-yl)-aminocarbonyl]-2-methyl-1-oxo-1,2,3,4-tetrahydroisoquinoline;
3-(3,5-bis-trifluoromethylphenyl)-4-[(3-methylpyrid-6-yl)-aminocarbonyl]-2-methyl-1-oxo-1,2,3,4-tetrahydroisoquinoline;
3-(3,5-bis-trifluoromethylphenyl)-4-[(4-methylpyrid-2-yl)-aminocarbonyl]-2-methyl-1-oxo-1,2,3,4-tetrahydroisoquinoline;
3-(3,5-bis-trifluoromethylphenyl)-4-[(pyrimidin-2-yl)-aminocarbonyl]-2-methyl-1-oxo-1,2,3,4-tetrahydroisoquinoline;
3-(3,5-bis-trifluoromethylphenyl)-4-{[2-(4-methoxyphenyl)ethyl]-aminocarbonyl}-2-(ethoxycarbonylethyl)-1-oxo-1,2,3,4-tetrahydroisoquinoline;
3-(3,5-bis-trifluoromethylphenyl)-4-[(N-benzylpiperidin-4-yl)-aminocarbonyl]-2-(aminocarbonylethyl)-1-oxo-1,2,3,4-tetrahydroisoquinoline;
3-(3,5-bis-trifluoromethylphenyl)-4-[(N-benzylpyrrolid-3-yl)-aminocarbonyl]-2-(aminocarbonylethyl)-1-oxo-1,2,3,4-tetrahydroisoquinoline;
3-(3,5-bis-trifluoromethylphenyl)-4-[(2-phenoxyethyl)-aminocarbonyl]-2-(aminocarbonylethyl)-1-oxo-1,2,3,4-tetrahydroisoquinoline;
3-(3,5-bis-trifluoromethylphenyl)-4-[(benzyl)-aminocarbonyl]-2-{[2-(3-methoxyphenyl)ethyl]aminocarbonylethyl}-1-oxo-1,2,3,4-tetrahydroisoquinoline;
3-(3,5-bis-trifluoromethylphenyl)-4-[(benzyl)-aminocarbonyl]-2-(tetrahydrofuran-2-ylmethyl)-1-oxo-1,2,3,4-tetrahydroisoquinoline;
3-(3,5-bis-trifluoromethylphenyl)-4-[(benzyl)-aminocarbonyl]-2-[(4-methylpiperidin-1-yl)carbonylethyl]-1-oxo-1,2,3,4-tetrahydroisoquinoline;
3-(3,5-bis-trifluoromethylphenyl)-4-[(benzyl)-aminocarbonyl]-2-(thiomorpholin-4-ylcarbonylethyl)-1-oxo-1,2,3,4-tetrahydroisoquinoline;
3-(3,5-bis-trifluoromethylphenyl)-4-[(2-methoxyethyl)-aminocarbonyl]-2-[2-(3-methoxyphenyl)ethylaminocarbonylethyl]-1-oxo-1,2,3,4-tetrahydroisoquinoline;
3-(3,5-bis-trifluoromethylphenyl)-4-[(furan-2-ylmethyl)-aminocarbonyl]-2-(4-acetylpiperazin-1-ylcarbonylethyl)-1-oxo-1,2,3,4-tetrahydroisoquinoline;
3-(3,5-bis-trifluoromethylphenyl)-4-[(furan-2-ylmethyl)-aminocarbonyl]-2-(2-methoxyethyl)-1-oxo-1,2,3,4-tetrahydroisoquinoline;
3-(3,5-bis-trifluoromethylphenyl)-4-[(cyclohexylmethyl)-aminocarbonyl]-1-oxo-1,2,3,4-tetrahydroisoquinoline;
3-(3,5-bis-trifluoromethylphenyl)-4-[(2-methoxyethyl)-aminocarbonyl]-2-(4-methoxyphenylmethyl)-1-oxo-1,2,3,4-tetrahydroisoquinoline;
3-(3,5-bis-trifluoromethylphenyl)-4-[(cyclopropylmethyl)-aminocarbonyl]-2-(4-methoxyphenylmethyl)-1-oxo-1,2,3,4-tetrahydroisoquinoline;
3-(3,5-bis-trifluoromethylphenyl)-4-[(2,3-dihydroxypropyl)-aminocarbonyl]-2-methyl-1-oxo-1,2,3,4-tetrahydroisoquinoline;
3-(3,5-bis-trifluoromethylphenyl)-4-[(methoxy)-aminocarbonyl]-2-methyl-1-oxo-1,2,3,4-tetrahydroisoquinoline;
3-(3,5-bis-trifluoromethylphenyl)-4-[(cyclopentyl)-aminocarbonyl]-2-(methoxycarbonylpentyl)-1-oxo-1,2,3,4-tetrahydroisoquinoline;
3-(3,5-bis-trifluoromethylphenyl)-4-[(2-aminoethyloxyethyloxyethyl)-aminocarbonyl]-2-methyl-1-oxo-1,2,3,4-tetrahydroisoquinoline;
3-(3,5-bis-trifluoromethylphenyl)-4-[(2-carboxy-3-methylbutyl)-aminocarbonyl]-2-methyl-1-oxo-1,2,3,4-tetrahydroisoquinoline;
3-(3,5-bis-trifluoromethylphenyl)-4-{[2-hydroxy-1,1-(dihydroxymethyl)ethyl]-aminocarbonyl}-2-methyl-1-oxo-1,2,3,4-tetrahydroisoquinoline;
3-(3,5-bis-trifluoromethylphenyl)-4-[(3-aminocarbonyl-1-carboxypropyl)-aminocarbonyl]-2-methyl-1-oxo-1,2,3,4-tetrahydroisoquinoline;
3-(3,5-bis-trifluoromethylphenyl)-4-[(1-carboxy-2-phenylethyl)-aminocarbonyl]-2-methyl-1-oxo-1,2,3,4-tetrahydroisoquinoline;
3-(3,5-bis-trifluoromethylphenyl)-4-[(N-methylpyrrol-2-ylmethyl)-aminocarbonyl]-2-methyl-1-oxo-1,2,3,4-tetrahydroisoquinoline;
3-(3,5-bis-trifluoromethylphenyl)-4-[(1,2-dimethylpyrrol-5-ylmethyl)-aminocarbonyl]-1-oxo-1,2,3,4-tetrahydroisoquinoline;
3-(3,5-bis-trifluoromethylphenyl)-4-[(2,3-dimethoxyphenylmethyl)-aminocarbonyl]-2-methyl-1-oxo-1,2,3,4-tetrahydroisoquinoline;
3-(3,5-bis-trifluoromethylphenyl)-4-[(2,4-difluorophenylmethyl)-aminocarbonyl]-2-methyl-1-oxo-1,2,3,4-tetrahydroisoquinoline;
3-(3,5-bis-trifluoromethylphenyl)-4-{[3-(difluoromethoxy)phenylmethyl]-aminocarbonyl3-2-methyl-1-oxo-1,2,3,4-tetrahydroisoquinoline;
3-(3,5-bis-trifluoromethylphenyl)-4-[(imidazol-1-ylpropyl)-aminocarbonyl]-2-methyl-1-oxo-1,2,3,4-tetrahydroisoquinoline;
3-(3,5-bis-trifluoromethylphenyl)-4-{[2-(methylsulfinyl)ethyl]-aminocarbonyl}-2-methyl-1-oxo-1,2,3,4-tetrahydroisoquinoline;
3-(3,5-bis-trifluoromethylphenyl)-4-[(methylsulfonylethyl)-aminocarbonyl]-2-methyl-1-oxo-1,2,3,4-tetrahydroisoquinoline;
3-(3,5-bis-trifluoromethylphenyl)-4-[(2-amino-4-oxo-3H-pyrimidin-6-yl)-aminocarbonyl]-2-methyl-1-oxo-1,2,3,4-tetrahydroisoquinoline;
3-(3,5-bis-trifluoromethylphenyl)-4-[(4-chloro-2-hydroxyphenyl)-aminocarbonyl]-1-oxo-1,2,3,4-tetrahydroisoquinoline;
3-(3,5-bis-trifluoromethylphenyl)-4-[(2-cyanophenyl)-aminocarbonyl]-1-oxo-1,2,3,4-tetrahydroisoquinoline;
3-(3,5-bis-trifluoromethylphenyl)-4-[(3-methoxycarbonylfuran-2-ylmethyl)-aminocarbonyl]-2-methyl-1-oxo-1,2,3,4-tetrahydroisoquinoline;
3-(3,5-bis-trifluoromethylphenyl)-4-{(5-mercapto-[1,3,4]-thiadiazol-2-yl)-aminocarbonyl}-1-oxo-1,2,3,4-tetrahydroisoquinoline;
3-(3,5-bis-trifluoromethylphenyl)-4-[(5-amino-1-carboxypentyl)-aminocarbonyl]-2-methyl-1-oxo-1,2,3,4-tetrahydroisoquinoline;
3-(3,5-bis-trifluoromethylphenyl)-4-[(5-amino-1-aminocarbonylpentyl)-aminocarbonyl]-2-methyl-1-oxo-1,2,3,4-tetrahydroisoquinoline;
3-(3,5-bis-trifluoromethylphenyl)-4-[(5,8-diphenyl-[1,2,4]triazocin-3-yl)-aminocarbonyl]-2-methyl-1-oxo-1,2,3,4-tetrahydroisoquinoline;
3-(3,5-bis-trifluoromethylphenyl)-4-[(6-ethoxybenzothiazol-2-yl)-aminocarbonyl]-2-methyl-1-oxo-1,2,3,4-tetrahydroisoquinoline;
3-(3,5-bis-trifluoromethylphenyl)-4-[(guanidino)-aminocarbonyl]-2-methyl-1-oxo-1,2,3,4-tetrahydroisoquinoline;
3-(3,5-bis-trifluoromethylphenyl)-4-[(3,5-dimethylisoxazol-4-yl)-aminocarbonyl]-2-methyl-1-oxo-1,2,3,4-tetrahydroisoquinoline;
3-(3,5-bis-trifluoromethylphenyl)-4-[(piperidin-4-yl)-aminocarbonyl]-2-methyl-1-oxo-1,2,3,4-tetrahydroisoquinoline;
3-(3,5-bis-trifluoromethylphenyl)-4-[(1,3-pyrazol-5-yl)-aminocarbonyl]-2-methyl-1-oxo-1,2,3,4-tetrahydroisoquinoline.

II. Representative compounds of Formula I, where R², R^{3′}, R⁶ and R⁷ are hydrogen, are disclosed in Table II. The stereochemistry at *C and **C of compounds prepared using methods described herein may be determined using standard analytical techniques known to one of ordinary skill in the art.

TABLE II

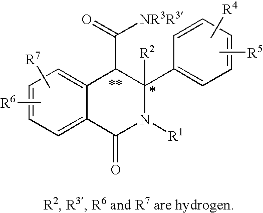

| Cpd | | | | |
|---|---|---|---|---|
| No. | R¹ | R³ | R⁴ | R⁵ |
| 150 | CH₃ | furan-2-ylmethyl | H | 3-SCF₃ |
| 151 | CH₃ | 2-methoxyethyl | H | 3-SCF₃ |
| 152 | CH₃ | cyclopentyl | H | 3-SCF₃ |
| 153 | CH₃ | furan-2-ylmethyl | 3-CH₃ | 5-CH₃ |
| 154 | CH₃ | cyclopentyl | 3-CH₃ | 5-CH₃ |
| 155 | OH | cyolopropylmethyl | 3-CH₃ | 5-CH₃ |
| 156 | CH₃ | 2-methoxyethyl | 3-Cl | 5-Cl |
| 157 | CH₃ | furan-2-ylmethyl | 3-CF₃ | H |
| 158 | CH₃ | cyclopentyl | 3-CF₃ | H |
| 159 | CH₃ | 2-methoxyethyl | 3-CF₃ | H |
| 160 | CH₃ | 2-methoxyethyl | 3-OCF₃ | H |
| 161 | CH₃ | furan-2-ylmethyl 3-OCF₃ | H | |
| 162 | CH₃ | thiazol-2-yl | 3-OCF₃ | H |
| 163 | CH₃ | thiazol-2-yl | 2-CF₃ | 4-CF₃ |
| 164 | CH₃ | thiophen-2-ylmethyl | 2-CF₃ | 4-CF₃ |
| 165 | CH₃ | furan-2-ylmethyl | 3-Br | 5-Br |
| 166 | CH₃ | 2,4-dimethylpyrid-6-yl | H | H |
| 167 | CH₃ | pyrazol-3-yl | H | H |
| 168 | H | 2-methoxyethyl | H | H |

and are named as
3-(3-trifluoromethylthiophenyl)-4-[(furan-2-ylmethyl)-aminocarbonyl]-2-methyl-1-oxo-1,2,3,4-tetrahydroisoquinoline;
3-(3-trifluoromethylthiophenyl)-4-[(2-methoxyethyl)-aminocarbonyl]-2-methyl-1-oxo-1,2,3,4-tetrahydroisoquinoline;
3-(3-trifluoromethylthiophenyl)-4-[(cyclopentyl)-aminocarbonyl]-2-methyl-1-oxo-1,2,3,4-tetrahydroisoquinoline;
3-(3,5-dimethylphenyl)-4-[(furan-2-ylmethyl)-aminocarbonyl]-2-methyl-1-oxo-1,2,3,4-tetrahydroisoquinoline;
3-(3,5-dimethylphenyl)-4-[(cyclopentyl)-aminocarbonyl]-2-methyl-1-oxo-1,2,3,4-tetrahydroisoquinoline;
3-(3,5-dimethylphenyl)-4-[(cyclopropylmethyl)-aminocarbonyl]-2-hydroxy-1-oxo-1,2,3,4-tetrahydroisoquinoline;
3-(3,5-dichlorophenyl)-4-[(2-methoxyethyl)-aminocarbonyl]-2-methyl-1-oxo-1,2,3,4-tetrahydroisoquinoline;
3-(3-trifluoromethylphenyl)-4-[(furan-2-ylmethyl)-aminocarbonyl]-2-methyl-1-oxo-1,2,3,4-tetrahydroisoquinoline;
3-(3-trifluoromethylphenyl)-4-[(cyclopentyl)-aminocarbonyl]-2-methyl-1-oxo-1,2,3,4-tetrahydroisoquinoline;
3-(3-trifluoromethylphenyl)-4-[(2-methoxyethyl)-aminocarbonyl]-2-methyl-1-oxo-1,2,3,4-tetrahydroisoquinoline;
3-(3-trifluoromethoxyphenyl)-4-[(2-methoxyethyl)-aminocarbonyl]-2-methyl-1-oxo-1,2,3,4-tetrahydroisoquinoline;
3-(3-trifluoromethoxyphenyl)-4-[(furan-2-ylmethyl)-aminocarbonyl]-2-methyl-1-oxo-1,2,3,4-tetrahydroisoquinoline;
3-(3-trifluoromethoxyphenyl)-4-[(thiazol-2-yl)-aminocarbonyl]-2-methyl-1-oxo-1,2,3,4-tetrahydroisoquinoline;
3-(2,4-bis-trifluoromethylphenyl)-4-[(thiazol-2-yl)-aminocarbonyl]-2-methyl-1-oxo-1,2,3,4-tetrahydroisoquinoline;
3-(2,4-bis-trifluoromethylphenyl)-4-[(thiophen-2-ylmethyl)-aminocarbonyl]-2-methyl-1-oxo-1,2,3,4-tetrahydroisoquinoline;
3-(3,5-dibromophenyl)-4-[(furan-2-ylmethyl)-aminocarbonyl]-2-methyl-1-oxo-1,2,3,4-tetrahydroisoquinoline;
3-phenyl-4-[(2,4-dimethylpyrid-6-yl)-aminocarbonyl]-2-methyl-1-oxo-1,2,3,4-tetrahydroisoquinoline;
3-phenyl-4-[(pyrazol-3-yl)-aminocarbonyl]-2-methyl-1-oxo-1,2,3,4-tetrahydroisoquinoline;
3-phenyl-4-[(2-methoxyethyl)-aminocarbonyl]-1-oxo-1,2,3,4-tetrahydroisoquinoline.

III. Representative compounds of Formula I, where R² and R^{3′} are hydrogen and where R⁴ and R⁵ are trifluoromethyl, are disclosed in Table III. The stereochemistry at *C and **C of compounds prepared using methods described herein may be determined using standard analytical techniques known to one of ordinary skill in the art.

TABLE III

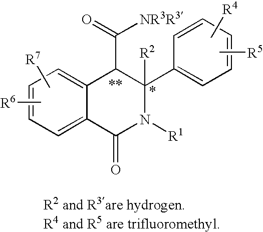

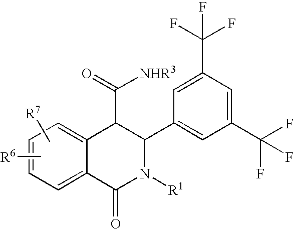

| Cpd | | | | |
|---|---|---|---|---|
| No. | R¹ | R³ | R⁶ | R⁷ |
| 169 | CH₃ | furan-2-ylmethyl | 7-OCH₃ | H |
| 170 | H | furan-2-ylmethyl | 6-OCH₃ | 7-OCH₃ |
| 171 | CH₃ | pyridin-2-ylmethyl 7-CH₃ | H | |
| 172 | CH₃ | furan-2-ylmethyl | 7-CH₃ | H |
| 173 | CH₃ | thiazol-2-ylmethyl | 7-CH₃ | H |
| 174 | OH | furan-2-ylmethyl | 6-OCH₃ | 7-OCH₃ |
| 175 | CH₃ | 2-methoxyethyl | 7-CH₃ | H |
| 176 | CH₃ | 2-methoxyethyl | 7-OCH₃ | H |
| 177 | CH₃ | cyclopropylmethyl | 6-CH₃ | H |
| 178 | CH₃ | furan-2-ylmethyl | 6-OCH₃ | 7-OCH₃ |
| 179 | CH₃ | thiazol-2-yl | 6-OCH₃ | 7-OCH₃ |
| 180 | H | cyclopropylmethyl | 6-OCH₃ | 7-OCH₃ |
| 181 | CH₃ | pyrid-4-ylmethyl | H | 7-OCH₃ |
| 182 | CH₃ | piperidin-4-ylmethyl | H | 7-OCH₃ |
| 183 | CH₃ | furan-2-ylmethyl | 6-CH₃ | H |
| 184 | CH₃ | 4,5-dihydro-thiazol-2-yl | 6-CH₃ | H |
| 185 | CH₃ | cyclopropylmethyl | 6-Cl | H |
| 186 | CH₃ | cyclopentyl | H | 7-Cl |
| 187 | CH₃ | 5-methylfuran-2-ylmethyl | H | 7-Cl |

and are named as
3-(3,5-bis-trifluoromethylphenyl)-4-[(furan-2-ylmethyl)-aminocarbonyl]-7-methoxy-2-methyl-1-oxo-1,2,3,4-tetrahydroisoquinoline;
3-(3,5-bis-trifluoromethylphenyl)-4-[(furan-2-ylmethyl)-aminocarbonyl]-6,7-dimethoxy-1-oxo-1,2,3,4-tetrahydroisoquinoline;
3-(3,5-bis-trifluoromethylphenyl)-4-[(pyridin-2-ylmethyl)-aminocarbonyl]-2,7-dimethyl-1-oxo-1,2,3,4-tetrahydroisoquinoline;
3-(3,5-bis-trifluoromethylphenyl)-4-[(furan-2-ylmethyl)-aminocarbonyl]-2,7-dimethyl-1-oxo-1,2,3,4-tetrahydroisoquinoline;
3-(3,5-bis-trifluoromethylphenyl)-4-[(thiazol-2-ylmethyl)-aminocarbonyl]-2,7-dimethyl-1-oxo-1,2,3,4-tetrahydroisoquinoline;
3-(3,5-bis-trifluoromethylphenyl)-4-[(furan-2-ylmethyl)-aminocarbonyl]-6,7-dimethoxy-2-hydroxy-1-oxo-1,2,3,4-tetrahydroisoquinoline;
3-(3,5-bis-trifluoromethylphenyl)-4-[(2-methoxyethyl)-aminocarbonyl]-2,7-dimethyl-1-oxo-1,2,3,4-tetrahydroisoquinoline;
3-(3,5-bis-trifluoromethylphenyl)-4-[(2-methoxyethyl)-aminocarbonyl]-7-methoxy-2-methyl-1-oxo-1,2,3,4-tetrahydroisoquinoline;
3-(3,5-bis-trifluoromethylphenyl)-4-[(cyclopropylmethyl)-aminocarbonyl]-2,6-dimethyl-1-oxo-1,2,3,4-tetrahydroisoquinoline;
3-(3,5-bis-trifluoromethylphenyl)-4-[(furan-2-ylmethyl)-aminocarbonyl]-6,7-dimethoxy-2-methyl-1-oxo-1,2,3,4-tetrahydroisoquinoline;
3-(3,5-bis-trifluoromethylphenyl)-4-[(thiazol-2-yl)-aminocarbonyl]-6,7-dimethoxy-2-methyl-1-oxo-1,2,3,4-tetrahydroisoquinoline;
3-(3,5-bis-trifluoromethylphenyl)-4-[(cyclopropylmethyl)-aminocarbonyl]-6,7-dimethoxy-1-oxo-1,2,3,4-tetrahydroisoquinoline;
3-(3,5-bis-trifluoromethylphenyl)-4-[(pyrid-4-ylmethyl)-aminocarbonyl]-7-methoxy-2-methyl-1-oxo-1,2,3,4-tetrahydroisoquinoline;
3-(3,5-bis-trifluoromethylphenyl)-4-[(piperidin-4-ylmethyl)-aminocarbonyl]-7-methoxy-2-methyl-1-oxo-1,2,3,4-tetrahydroisoquinoline;
3-(3,5-bis-trifluoromethylphenyl)-4-[(furan-2-ylmethyl)-aminocarbonyl]-2,6-dimethyl-1-oxo-1,2,3,4-tetrahydroisoquinoline;
3-(3,5-bis-trifluoromethylphenyl)-4-[(4,5-dihydro-thiazol-2-yl)-aminocarbonyl]-2,6-dimethyl-1-oxo-1,2,3,4-tetrahydroisoquinoline;
3-(3,5-bis-trifluoromethylphenyl)-4-[(cyclopropylmethyl)-aminocarbonyl]-6-chloro-2-methyl-1-oxo-1,2,3,4-tetrahydroisoquinoline;
3-(3,5-bis-trifluoromethylphenyl)-4-[(cyclopentyl)-aminocarbonyl]-7-chloro-2-methyl-1-oxo-1,2,3,4-tetrahydroisoquinoline;
3-(3,5-bis-trifluoromethylphenyl)-4-[(5-methylfuran-2-ylmethyl)-aminocarbonyl]-7-chloro-2-methyl-1-oxo-1,2,3,4-tetrahydroisoquinoline.

IV. Representative compounds of Formula I, where R² is hydrogen and where R⁴ and R⁵ are trifluoromethyl, are disclosed in Table IV. The stereochemistry at *C and **C of compounds prepared using methods described herein may be determined using standard analytical techniques known to one of ordinary skill in the art.

TABLE IV

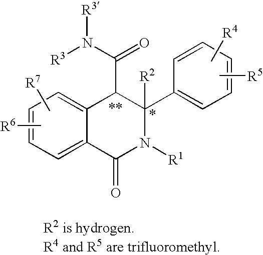

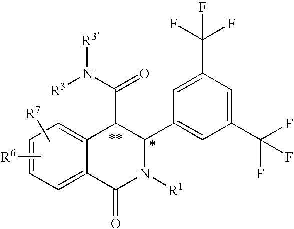

| Cpd | | | | |
|---|---|---|---|---|
| No. | R¹ | —NR³R^{3′} | R⁶ | R⁷ |
| 188 | aminocarbonylethyl | 3,5-dimethylmorpholin- | H | H |
| | | 4-yl | | |
| 189 | aminocarbonylethyl | 4-acetylpiperazin-1-yl | H | H |
| 190 | CH₃ | 4-acetylpiperazin-1-yl | H | H |
| 191 | 2-methoxyethyl | 4-acetylpiperazin-1-yl | H | H |
| 192 | CH₃ | piperazin-1-yl | H | H |
| 193 | CH₃ | morpholin-4-y1 | H | H |
| 194 | CH₃ | piperazin-1-yl | 6-OCH₃ | 7-OCH₃ |
| 195 | CH₃ | morpholin-4-yl | 6-OCH₃ | 7-OCH₃ |
| 196 | CH₃ | N-methyl-N-(2-pyrid-4- | H | H |
| | | ylethyl)amino | | |
| 197 | CH₃ | N-methyl-N-(furan-2- | H | H |
| | | ylmethyl)amino | | |
| 198 | CH₃ | 3-aminopyrazol-1-yl | H | H |

and are named as
3-(3,5-bis-trifluoromethylphenyl)-4-[(3,5-dimethylmorpholin-4-yl)-carbonyl]-2-(aminocarbonylethyl)-1-oxo-1,2,3,4-tetrahydroisoquinoline;
3-(3,5-bis-trifluoromethylphenyl)-4-[(4-acetylpiperazin-1-yl)-carbonyl]-2-(aminocarbonylethyl)-1-oxo-1,2,3,4-tetrahydroisoquinoline;
3-(3,5-bis-trifluoromethylphenyl)-4-[(4-acetylpiperazin-1-yl)-carbonyl]-2-methyl-1-oxo-1,2,3,4-tetrahydroisoquinoline;
3-(3,5-bis-trifluoromethylphenyl)-4-[(4-acetylpiperazin-1-yl)-carbonyl]-2-(2-methoxyethyl)-1-oxo-1,2,3,4-tetrahydroisoquinoline;
3-(3,5-bis-trifluoromethylphenyl)-4-[(piperazin-1-yl)-carbonyl]-2-methyl-1-oxo-1,2,3,4-tetrahydroisoquinoline;
3-(3,5-bis-trifluoromethylphenyl)-4-[(morpholin-4-yl)-carbonyl]-2-methyl-1-oxo-1,2,3,4-tetrahydroisoquinoline;
3-(3,5-bis-trifluoromethylphenyl)-6,7-dimethoxy4-[(piperazin-1-yl)-carbonyl]-2-methyl-1-oxo-1,2,3,4-tetrahydroisoquinoline;
3-(3,5-bis-trifluoromethylphenyl)-6,7-dimethoxy-4-[(morpholin-4-yl)-carbonyl]-2-methyl-1-oxo-1,2,3,4-tetrahydroisoquinoline;
3-(3,5-bis-trifluoromethylphenyl)-4-{[N-methyl-N-(2-pyrid-4-ylethyl)]-aminocarbonyl}-2-methyl-1-oxo-1,2,3,4-tetrahydroisoquinoline;
3-(3,5-bis-trifluoromethylphenyl)-4-{[N-methyl-N-(furan-2-ylmethyl)]-aminocarbonyl}-2-methyl-1-oxo-1,2,3,4-tetrahydroisoquinoline;
3-(3,5-bis-trifluoromethylphenyl)-4-[(3-aminopyrazol-1-yl)-carbonyl]-2-methyl-1-oxo-1,2,3,4-tetrahydroisoquinoline.

### General Synthesis

Compounds of this invention can be made by the methods depicted in the reaction schemes shown below.

The starting materials and reagents used in preparing these compounds are either available from commercial suppliers such as Aldrich Chemical Co. (Milwaukee, Wis.), Bachem (Torrance, Calif.), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4^{th} Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to filtration, distillation, crystallization, chromatography and the like. Such materials may be characterized using conventional means, including physical constants and spectral data. In particular stereochemistry of isomers may be determined by analytical methods known to one of ordinary skill in the art.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

Compounds of Formula I can be prepared by the procedure illustrated and described in Scheme A below.

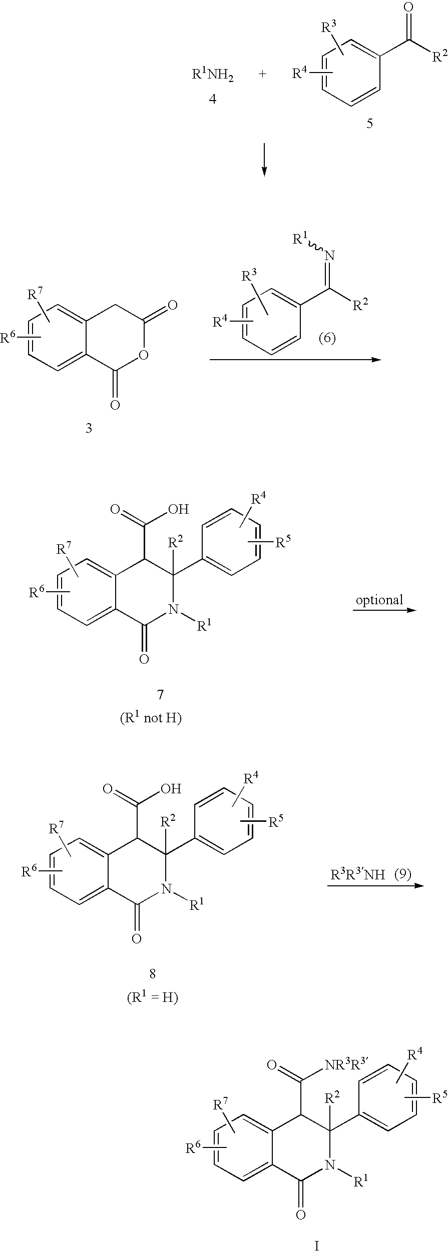

Compounds of formula 3, 4 and 5 are commercially available or they can be prepared by methods well known in the art. For example, see C. Weimer et. al., Archivc der Pharmazie, 324(8), 1991, 509-518. Reaction of a homophthalic anhydride of formula 1 (where R⁶ and R⁷ are as defined in the Summary of the invention except where amino group) with an imine intermediate of formula 6, prepared by reacting an amine R¹NH₂ of formula 4 (where R¹ is as defined in the Summary of the Invention except hydrogen) with a benzaldehyde (R² is hydrogen) or acetophenone (R² is methyl) of formula 5, gives a mixture of cis/trans-1-oxo-3-phenyl-1,2,3,4-tetrahydroisoquinoline4-carboxylic acid of formula 7. The reaction is carried out in an inert organic solvent such as methylene chloride, and the like. The individual diastereomers can be isolated, if desired, by methods well known in the art.

A compound of formula 7 prepared above, where R¹ is a group such as benzyl or substituted benzyl can be converted to a corresponding compound of formula 8 where R¹ is hydrogen, if desired, by removal of the R¹ group using methods known to one skilled in the art.

Reaction of a compound of formula 7 or 8 with an amine R³R^{3′}NH of formula 9 where R³ and R^{3′} are as defined in the Summary of the invention then provides a compound of Formula I. The reaction is carried out in the presence of a coupling agent such as benzotriazol-1-yloxytrispyrrolidinophosphonium hexafluorophosphate (PyBOP) or O-(7-azabenzotriazol-1-yl)-N,N,N′,N′-tetramethyluronium hexa fluorophosphate (HATU), and the like, and a non-nucleophilic organic amine such as N,N-diisopropylethylamine, triethylamine, or pyridine, and the like. The reaction is carried out in a suitable organic solvent such as dichloromethane, chloroform, or tetrahydrofuran, and the like. Instead of using coupling agents, compounds of Formula I can also be prepared by conversion of acids 7 or 8 into acid chlorides using methods known in the art followed by reaction with an amine R³R^{3′}NH of formula 9 in the presence of an organic base such as N,N-diisopropylethylamine or NaH, and the like, in organic solvents such as dimethylacetamide or dimethylformamide, and the like.

### Utility

The compounds of this invention are activators of caspases and inducers of apoptosis and are therefore useful in the treatment of a disease in which caspase cascade mediated physiological responses are implicated. In particular the compounds of this invention are useful in the treatment of proliferative diseases such as cancer which includes, but are not limited to, Hodgkin's disease, non-Hodgkin's lymphomas, acute and chronic lymphocytic leukemias, multiple myeloma, neuroblastoma, breast carcinomas, ovarian carcinomas, lung carcinomas, Wilms' tumor, cervical carcinomas, testicular carcinomas, soft tissue sarcomas, chronic lymphocytic leukemia, primary macroglobulinemia, bladder carcinomas, chronic granulocytic leukemia, primary brain carcinomas, malignant melanoma, small-cell lung carcinomas, stomach carcinomas, colon carcinomas, malignant pancreatic insulinoma, malignant carcinoid carcinomas, malignant melanomas, choriocarcinomas, mycosis fungoides, head and neck carcinomas, osteogenic sarcoma, pancreatic carcinomas, acute granulocytic leukemia, hairy cell leukemia, neuroblastoma, rhabdomyo sarcoma, Kaposi's sarcoma, genitourinary carcinomas, thyroid carcinomas, esophageal carcinomas, malignant hypercalcemia, cervical hyperplasia, renal cell carcinomas, endometrial carcinomas, polycythemia vera, essential thrombocytosis, adrenal cortex carcinomas, skin cancer, and prostatic carcinomas.

A wide range of immune mechanisms operate rapidly following exposure to an infectious agent. Depending on the type of infection, rapid clonal expansion of the T and B lymphocytes occurs to combat the infection. The elimination of the effector cells following an infection is one of the major mechanisms maintaining immune homeostasis. This deletion of reactive cell has been shown to be regulated by a phenomenon known as apoptosis. Autoimmune diseases have been lately identified as a consequence of deregulated cell death. In certain autoimmune diseases, the immune system directs its powerful cytotoxic effector mechanisms against specialized cells such as oligodendrocytes in multiple sclerosis, the beta cells of the pancreas in diabetes mellitus, and thyrocytes in Hashimoto's thyroiditis (Ohsako. S. & Elkon, K. B., Cell Death Differ. 1999, 6, 13-21). Mutations of the gene encoding the lymphocyte apoptosis receptor Fas/APO-1/CD95 are reported to be associated with defective lymphocyte apoptosis and autoimmune lymphoproliferative syndrome (ALPS), which is characterized by chronic, histologically benign splenomegaly and generalized lymphadenopathy, hypergammaglobulinemia, and autoantibody formation (Infante, A. J., et al., J Pediatr. 1998, 133 629-633 and Vaishnaw, A. K., et al., J Clin. Invest. 1999, 103, 355-363).

Overexpression of Bcl-2, which is a member of the bcl-2 gene family of programmed cell death regulators with anti-apoptotic activity in developing B cells of transgenic mice, in the presence of T cell dependent co-stimulatory signals, results in the generation of a modified B cell repertoire and in the production of pathogenic autoantibodies (Lopez-Hoyos, M., et al., Int. J Mol. Med. 1998, 1, 475483).

Accordingly, many types of autoimmune disease may be caused by defects of the apoptotic process, and one treatment strategy would be to turn on apoptosis in the lymphocytes that are causing autoimmune disease (O'Reilly, L. A. & Strasser, A., Inflamm. Res. 1999, 48, 5-21).

Fas-Fas ligand (FasL) interaction is known to be required for the maintenance of immune homeostasis. Experimental autoimmune thyroiditis (EAT), characterized by autoreactive T and B cell responses and a marked lymphocytic infiltration of the thyroid, is a good model to study the therapeutic effects of FasL. Batteux, F., et al., (J. Immunol. 1999, 162, 603-608) reported that by direct injection of DNA expression vectors encoding FasL into the inflamed thyroid, the development of lymphocytic infiltration of the thyroid was inhibited and induction of infiltrating T cells death was observed. These results show that FasL expression on thyrocytes may have a curative effect on ongoing EAT by inducing death of pathogenic autoreactive infiltrating T lymphocytes.

Bisindolylmaleimide VII is known to potentiate Fas-mediated apoptosis in human astrocytoma 1321NI cells and in Molt-4T cells, and both of which were resistant to apoptosis induced by anti-Fas antibody in the absence of bisindolylmaleimide VIII. Potentiation of Fas-mediated apoptosis by bisindolylmaleimide VII was reported to be selective for activated, rather than non-activated, T cells, and was Fas-dependent. Zhou T., el al., (Nat. Med 5:42-49 (1999)) reported that administration of bisindolylmaleimide VIII to rats during autoantigen stimulation prevented the development of symptoms of T cell-mediated autoimmune diseases in two models, the Lewis rat model of experimental allergic encephalitis and the Lewis adjuvant arthritis model. Therefore the application of a Fas-dependent apoptosis enhancer such as bisindolylmaleimide VIII may be therapeutically useful for the more effective elimination of detrimental cells and inhibition of T cell-mediated autoimmune diseases. Therefore the compounds of this invention should be an effective in the treatment of autoimmune diseases.

Psoriasis is a chronic skin disease that is characterized by scaly red patches. Psoralen plus ultraviolet A (PUVA) is a widely used and effective treatment for psoriasis vulgaris and Coven, et al., in Photodermatol. Photoimmunol. Photomed 1999, 15, 22-27, reported that lymphocytes treated with psoralen 8-MOP or TMP plus UVA displayed DNA degradation patterns typical of apoptotic cell death. Ozawa, et al. in J. Exp. Med 1999, 189, 711-718 reported that induction of T cell apoptosis could be the main mechanism by which 312-nm UVB resolves psoriasis skin lesions. Low doses of methotrexate may be used to treat psoriasis to restore a clinically normal skin. Heenen, et al. in Arch. Dermatol. Res. 1998, 290, 240-245 reported that low doses of methotrexate may induce apoptosis and this mode of action could explain the reduction in epidermal hyperplasia during treatment of psoriasis with methotrexate. Therefore the compounds of this invention which function as a caspase cascade activator and inducer of apoptosis, should be effective in the treatment of psoriasis.

Synovial cell hyperplasia is a characteristic of patients with rheumatoid arthritis (RA). Excessive proliferation of RA synovial cells as well as defects in synovial cell death might be responsible for the synovial cell hyperplasia. Wakisaka, et al., Clin. Exp. Immunol. 114:119-128 (1998), found that although RA synovial cells could die via apoptosis through Fas/FasL pathway, apoptosis of synovial cells was inhibited by proinflammatory cytokines present within the synovium, and suggested that inhibition of apoptosis by the proinflammatory cytokines may contribute to the outgrowth of synovial cells, and lead to pannus formation and the destruction of joints in patients with RA. Therefore the compounds of this invention which function as a caspase cascade activator and inducer of apoptosis should also be effective in the treatment of rheumatoid arthritis.

An accumulation of convincing evidence suggests that apoptosis plays a major role in promoting resolution of the acute inflammatory response. Neutrophils are constitutively programmed to undergo apoptosis, thus limiting their pro-inflammatory potential and leading to rapid, specific, and non-phlogistic recognition by macrophages and semi-professional phagocytes (Savill, J., J. Leukoc. Biol. 1997, 61, 375-380). Boirivant, et al. in Gastroenterology 1999, 116, 557-565 reported that lamina propria T cells isolated from areas of inflammation in Crohn's disease, ulcerative colitis, and other inflammatory states manifest decreased CD2 pathway-induced apoptosis, and that studies of cells from inflamed Crohn's disease tissue indicate that this defect is accompanied by elevated Bcl-2 levels. Therefore the compounds of this invention which function as a caspase cascade activator and inducer of apoptosis should also be effective in the treatment of inflammation and inflammatory bowel disease.

### Administration and Pharmaceutical Compositions

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors.

Therapeutically effective amounts of compounds of Formula I or Ia may range from approximately 0.1-50 mg per kilogram body weight of the recipient per day; preferably about 0.5-20 mg/kg/day. Thus, for administration to a 70 kg person, the dosage range would most preferably be about 35 mg to 1.4 g per day. If a known chemotherapeutic agent is also administered, it is administered in an amount which is effective to achieve its intended purpose. The amounts of such known cancer chemotherapeutic agents effective for cancer are well known to those of skill in the art.

In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral or parenteral using a convenient daily dosage regimen, which can be adjusted according to the degree of affliction. Oral compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailabihty based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of Formula I or Ia in combination with at least one pharmaceutically acceptable excipient. Acceptable excipients are non-toxic, aid administration, and do not adversely affect the therapeutic benefit of the compound of Formula I or Ia. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 18^{th} ed., 1990).

The amount of the compound in a formulation can vary within the full range employed by those skilled in the art. Typically, the formulation will contain, on a weight percent (wt %) basis, from about 0.01-99.99 wt % of a compound of Formula I or Ia based on the total formulation, with the balance being one or more suitable pharmaceutical excipients. Preferably, the compound is present at a level of about 1-80 wt %. Representative pharmaceutical formulations containing a compound of Formula I or Ia are described below.

As stated previously, the compounds of this invention can be administered in combination with known anti-cancer agents. Such known anti-cancer agents include the following: estrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors, and other angiogenesis inhibitors. The compound of the present invention compounds are particularly useful when adminsitered in combination with radiation therapy. Preferred angiogenesis inhibitors are selected from the group consisting of a tyrosine kinase inhibitor, an inhibitor of epidermal-derived growth factor, an inhibitor of fibroblast-derived growth factor, an inhibitor of platelet derived growth factor, an MMP (matrix metalloprotease) inhibitor, an integrin blocker, interferon-cc, interleukin-12, pentosan polysulfate, a cyclooxygenase inhibitor, carboxyamidotriazole, combretastatin A-4, squalamine, 6-O-chloroacetyl-carbonyl)-fumagillol, thalidomide, angiostatin, troponin-1, and an antibody to VEGF.

Preferred estrogen receptor modulators are tamoxifen and raloxifene.

“Estrogen receptor modulators” refers to compounds that interfere or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl) retinamide, and N-4-carboxyphenyl retinamide.

“Cytotoxic agents” refer to compounds which cause cell death primarily by interfering directly with the cell's functioning or inhibit or interfere with cell myosis, including alkylating agents, tumor necrosis factors, intercalators, microtubulin inhibitors, and topoisomerase inhibitors.

Examples of cytotoxic agents include, but are not limited to, tirapazimine, sertenef, cachectin, ifosfamide, tasonermin, lonidamine, carboplatin, altretamine, prednimustine, dibromodul citol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine) platinum, benzylguanine, glufosfamide, GPX100, (trans, trans, trans)-bis-mu-(hexane-1,6-diamine)-mu-[diamine-platinum(II)]bis[diamine(chloro)platinum(II)]tetrachloride, diarizidinylspermine, arsenic trioxide, 1-(11-dodecylamino-10-hydroxyundecyl)-3,7-dimethylxanthine, zorubicin, idarubicin, daunorubicin, mitoxantrone, pirarubicin, bisantrene, antineoplaston, amrubicin, valrubicin, pinafide, annamycin, 3′-deamino-3′-morpholino-13-deoxo-10-hydroxycarminomycin, galarubicin, elinafide, MEN10755, and 4-demethoxy-3-deamino-3-aziridinyl-4-methylsulphonyl-daunorubicin (see WO 00/50032).

Examples of microtubulin inhibitors include paclitaxel, vindesine sulfate, 3′,4′-didehydro4′-deoxy-8′-norvincaleukoblastine, docetaxol, rhizoxin, dolastatin, mivobulin isethionate, auristatin, cemadotin, RPR109881, BMS184476, cryptophycin, 2,3,4,5,6-pentafluoro-N-(3-fluoro4-methoxyphenyl)benzene sulfonamide, anhydrovinblastine, vinflunine, N,N-dimethyl-L-valyl-L-valyl-N-methyl-L-valyl-L-prolyl-L-proline-t-butylamide, TDX258, and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)-ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro(3′,4′:6,7)colchic(2,3-d)-1,3-dioxol-6-one, 2,3-(methylenedioxy)-5-methyl-7-hydroxy-8-methoxybenzo[c]-phenanthridinium, 6,9-bis[(2-aminoethyl)-amino]benzo[g]isoguinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, and dimesna.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]-adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-flurouracil, alanosine, 11-acetyl-8-(carbamoyloxymethyl)-4-formyl-6-methoxy-14-oxa-1,11-diazatetra cyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine, and 3-aminopyridine-2-carboxaldehyde thiosemicarbazone. “Antiproliferative agents” also includes monoclonal antibodies to growth factors, other than those listed under “angiogenesis inhibitors”, such as trastuzumab, and tumor suppressor genes, such as p53, which can be delivered via recombinant virus-mediated gene transfer (see U.S. Pat. No. 6,069,134, for example).

“HMG-CoA reductase inhibitors” refers to inhibitors of 3-hydroxy-3-methylglutaryl-CoA reductase. Compounds which have inhibitory activity for HMG-CoA reductase can be readily identified by using assays well-known in the art. For example, see the assays described or cited in U.S. Pat. No. 4,231,938 at col. 6, and WO 84/02131 at pp. 30-33. The terms “HMG-CoA reductase inhibitor” and “inhibitor of HMG-CoA reductase” have the same meaning when used herein. It has been reported that (Int. J. Cancer, 20;97(6):746-50, 2002) combination therapy with lovastatin, a HMG-CoA reductase inhibitor, and butyrate, an inducer of apoptosis in the Lewis lung carcinoma model in mice showed potentiating antitumor effects

Examples of HMG-CoA reductase inhibitors that may be used include but are not limited to lovastatin (MEVACOR®; see U.S. Pat. Nos. 4,231,938; 4,294,926; 4,319,039), simvastatin (ZOCOR®; see U.S. Pat. Nos. 4,444,784; 4,820,850; 4,916,239), pravastatin (PRAVACHOL®; see U.S. Pat. Nos. 4,346,227; 4,537,859; 4,410,629; 5,030,447 and 5,180,589), fluvastatin (LESCOL®; see U.S. Pat. Nos. 5,354,772; 4,911,165; 4,929,437; 5,189,164; 5,118,853; 5,290,946; 5,356,896), atorvastatin (LIPITOR®; see U.S. Pat. Nos. 5,273,995; 4,681,893; 5,489,691; 5,342,952) and cerivastatin (also known as rivastatin and BAYCHOL®; see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89 (Feb. 5, 1996) and U.S. Pat Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and □lolchicin the use of such salts, esters, open-acid and lactone forms is included within the scope of this invention.

In HMG-CoA reductase inhibitors where an open-acid form can exist, salt and ester forms may preferably be formed from the open-acid, and all such forms are included within the meaning of the term “HMG-CoA reductase inhibitor” as used herein. Preferably, the HMG-CoA reductase inhibitor is selected from lovastatin and simvastatin, and most preferably simvastatin.

Herein, the term “pharmaceutically acceptable salts” with respect to the EMG-CoA reductase inhibitor shall mean non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, omithine, choline, N,N′-dibenzylethylenediamine, chloroprocaine, diethanolamine, procaine, N-benzylphenethylamine, 1-p-chlorobenzyl-2-pyrrolidine-1′-yl-methylbenzimidazole, diethylamine, piperazine; and tris(hydroxymethyl) aminomethane. Further examples of salt forms of HMG-CoA reductase inhibitors may include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, and valerate.

Ester derivatives of the described HMG-CoA reductase inhibitor compounds may act as prodrugs which, when absorbed into the bloodstream of a warm-blooded animal, may cleave in such a manner as to release the drug form and permit the drug to afford improved therapeutic efficacy.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase). Examples of prenyl-protein transferase inhibiting compounds include (±)-6-[amino(4-chlorophenyl)(1-methyl-1H-imidazol-5-yl)methyl]4-(3-chlorophenyl)-1-methyl-2(1H)-quinolinone, (−)-6-[amino(4-chlorophenyl)(1-methyl-1H-imidazol-5-yl)methyl]-4-(3-chlorophenyl)-1-methyl-2(1H)-quinolinone, (+)-6-[amino(4-chlorophenyl)(1-methyl-1H-imidazol-5-yl)methyl]-4-(3-chloro phenyl)-1-methyl-2(1H)-quinolinone, 5(S)-n-butyl-1-(2,3-dimethylphenyl)-4-[1-(4-cyanobenzyl)-5-imidazolylmethyl]-2-piperazinone, (S)-1-(3-chlorophenyl)-4-[1-(4-cyanobenzyl)-5-imidazolylmethyl]-5-[2-(ethanesulfonyl)-methyl)-2-piperazinone, 5(S)-n-butyl-1-(2-methylphenyl)-4-[1-(4-cyanobenzyl)-5-imidazolylmethyl]-2-piperazinone, 1-(3-chlorophenyl) -4-[1-(4-cyanobenzyl)-2-methyl-5-imidazolylmethyl]-2-piperazinone, 1-(2,2-diphenylethyl)-3-[N-(1-(4-cyanobenzyl)-1H-imidazol-5-ylethyl)carbamoyl]piperidine, 4-{5-[4-hydroxymethyl-4-(4-chloropyridin-2-ylmethyl)-piperidine-1-ylmethyl]-2-methylimidazol-1-ylmethyl}benzonitrile, 4-{5-[4-hydroxymethyl-4-(3-chlorobenzyl)-piperidin-1-ylmethyl]-2-methylimidazol-1-ylmethyl}benzonitrile, 4-{3-[4-(2-oxo-2H-pyridin-1-yl)benzyl]-3H-imidazol-4-ylmethyl}benzonitrile, 4-{3-[4-(5-chloro-2-oxo-2H-[1,2′]bipyridin-5′-ylmethyl]imidazol-4-ylmethyl}benzonitrile, 4-{3-[4-(2-oxo-2H-[1,2′]bipyridin-5′-ylmethyl]imidazol-4-ylmethyl}benzonitrile, 4-[3-(2-oxo-1-phenyl-1,2-dihydropyridin-4-ylmethyl)-3H-imidazol-4-ylmethyl}benzonitrile, 18,19-dihydro-19-oxo-5H,17H-6,10:12,16-dimetheno-1H-imidazo[4,3-c][1,11,4]dioxa-azacyclononadecine-9-carbonitrile, (±)-19,20-dihydro-19-oxo-5H-18,21-ethano-12,14-etheno-6,10-metheno-22H-benzo[d]imidazo[4,3-k][1,6,9,12]-oxatriaza-cyclooctadecine-9-carbonitrile, 19,20-dihydro-19-oxo-5H,17H-18,21-ethano-6,10:12,16-dimetheno-22H-imidazo[3,4-h][1,8,11,14]oxatriazacyclo-eicosine-9-carbonitrile, and CD-19,20-dihydro-3-methyl-19-oxo-5H-18,21-ethano-12,14-etheno-6,10-metheno-22H-benzo[d]imidazo[4,3-k][1,6,9,12]oxa-triazacyclooctadecine-9-carbonitrile.

Other examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. Nos. 5,420,245, 5,523,430, 5,532,359, 5,510,510, 5,589,485, 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see J. of Cancer, Vol. 35, No. 9, pp.1394-1401 (1999).

Examples of HIV protease inhibitors include amprenavir, abacavir, CGP-73547, CGP-61755, DMP-450, indinavir, nelfinavir, tipranavir, ritonavir, saquinavir, ABT-378, AG 1776, and BMS-232, 632. Examples of reverse transcriptase inhibitors include delaviridine, efavirenz, GS-840, HB Y097, lamivudine, nevirapine, AZT, 3TC, ddC, and ddI. It has been reported (Nat. Med 2002, 8(3), 225-32) that HIV protease inhibitors, such as indinavir or saquinavir, have potent anti-angiogenic activities and promote regression of Kaposi sarcoma

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR20), inhibitors of epidermal-derived, fibroblast-derived, or platelet derived growth factors, MMP (matrix metalloprotease) inhibitors, integrin blockers, interferon-∝, interleukin-12, pentosan polysulfate, cyclooxygenase inhibitors, including nonsteroidal anti-inflammatories (NSAIDs) like aspirin and ibuprofen as well as selective cyclooxygenase-2 inhibitors like celecoxib, valecoxib, and rofecoxib (PNAS 1992, 89, 7384; JNCI 1982, 69, 475; Arch. Opthamol. 1990, 108, 573; Anat. Rec., 1994, 238, 68; FEBS Letters 1995, 372, 83; Clin. Orthop. 1995, 313, 76; J. Mol. Endocrinol. 1996, 16, 107; Jpn. J. Pharmacol. 1997, 75, 105; Cancer Res. 1997, 57, 1625; Cell 1998, 93, 705; Intl. J. Mol. Med. 1998, 2, 715; J. Biol. Chem. 1999, 274, 9116), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 1985, 105,141-145), and antibodies to VEGF (see Nature Biotechnology 1999, 17, 963-968; Kim et al., Nature 1993, 362, 841-844; WO 00/44777; and WO 00/61186).

As described above, the combinations with NSAID's are directed to the use of NSAID's which are potent COX-2 inhibiting agents. For purposes of this specification an NSAID is potent if it possess an IC₅₀ for the inhibition of COX-2 of 1 μM or less as measured by the cell or microsomal assay known in the art.

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC₅₀ for COX-2 over IC₅₀ for COX-1 evaluated by the cell or microsomal assay disclosed hereinunder. Such compounds include, but are not limited to those disclosed in U.S. Pat. No. 5,474,995, issued Dec. 12, 1995, U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued Jul. 25, 1995, U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. Pat. No. 5,698,584, issued Dec. 16, 1997, U.S. Pat. No. 5,710,140, issued Jan. 20, 1998, WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999, all of which are hereby incorporated by reference. Other examples of specific inhibitors of COX-2 include those disclosed in U.S. Pat. No. 6,313,138 the disclosure of which is incorporated herein by reference in its entirety.

General and specific synthetic procedures for the preparation of the COX-2 inhibitor compounds described above are found in U.S. Pat. No. 5,474,995, issued Dec. 12, 1995, U.S. Pat. No. 5,861,419, issued Jan. 19, 1999, and U.S. Pat. No. 6,001,843, issued Dec. 14, 1999, all of which are herein incorporated by reference.

Compounds that have been described as specific inhibitors of COX-2 and are therefore useful in the present invention include, but are not limited to, the following: 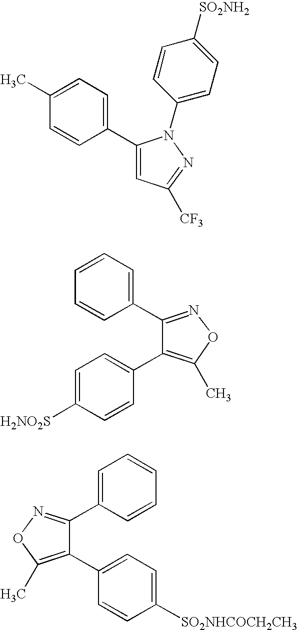
or a pharmaceutically acceptable salt thereof.

Compounds which are described as specific inhibitors of COX-2 and are therefore useful in the present invention, and methods of synthesis thereof, can be found in the following patents, pending applications and publications, which are herein incorporated by reference: WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999.

Compounds which are specific inhibitors of COX-2 and are therefore useful in the present invention, and methods of synthesis thereof, can be found in the following patents, pending applications and publications, which are herein incorporated by reference: U.S. Pat. No. 5,474,995, issued Dec. 12, 1995, U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued Jul. 25, 1995, U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. Pat. No. 5,698,584, issued Dec. 16, 1997, and U.S. Pat. No. 5,710,140, issued Jan. 20, 1998.

Other examples of angiogenesis inhibitors include, but are not limited to, endostatin, ukrain, ranpirnase, IM862, 5-methoxy4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]-methyl]-1H-1,2,3-triazole-4-carboxamide, CM101, squalamine, combretastatin, RPI4610, NX31838, sulfated mannopentose phosphate, 7,7-(carbonyl-bis[imino-N-methyl-4,2-pyrrolocarbonyl-imino[N-methyl4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counter-act binding of a physiological ligand to the αᵥβ₅ integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆; αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy -1H-diindolo[1,2,3-fg:3′,2′,1′-kl]pyrrolo[3,4-i][1,6]benzodiazocin-1-one, SH268, genistein, ST1571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, SU11248, ST1571A, N-4-chlorophenyl-4-(4-pyridylmethyl)-1-phthalazinamine, and EMD121974.

The instant compounds are also useful, alone or in combination with platelet fibrinogen receptor (GP IIb/IIIa) antagonists, such as tirofiban, to inhibit metastasis of cancerous cells. Tumor cells can activate platelets largely via thrombin generation. This activation is associated with the release of VEGF. The release of VEGF enhances metastasis by increasing extravasation at points of adhesion to vascular endothelium (Amirkhosravi, Platelets 1999, 10, 285-292). Therefore, the present compounds can serve to inhibit metastasis, alone or in combination with GP IIb/IIIa) antagonists. Examples of other fibrinogen receptor antagonists include abciximab, eptifibatide, sibrafiban, lamifiban, lotrafiban, cromofiban, and CT50352.

If formulated as a fixed dose, such combination products employ the compounds of this invention within the dosage range described above and the other pharmaceutically active agent(s) within its approved dosage range. Compounds of the instant invention may alternatively be used sequentially with known pharmaceutically acceptable agent(s) when a combination formulation is inappropriate.

The term administration and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

As used herein, the term “composition” is intended to encompass a product comprising the specified ingredients in the specified amounts, as well as any product which results, directly or indirectly, from combination of the specified ingredients in the specified amounts.

The compounds of the instant invention may also be co-administered with other well known therapeutic agents that are selected for their particular usefulness against the condition that is being treated. For example, the compounds of the instant invention may also be co-administered with other well known cancer therapeutic agents that are selected for their particular usefulness against the condition that is being treated. Included in such combinations of therapeutic agents are combinations of the farnesyl-protein transferase inhibitors disclosed in U.S. Pat. No. 6,313,138 and an antineoplastic agent It is also understood that such a combination of antineoplastic agent and inhibitor of farnesyl-protein transferase may be used in conjunction with other methods of treating cancer and/or tumors, including radiation therapy and surgery.

Examples of an antineoplastic agent include, in general, microtubule-stabilizing agents (such as paclitaxel (also known as Taxol®), docetaxel (also known as Taxoteresepothilone A, epothilone B, desoxyepothilone A, desoxyepothilone B or their derivatives); microtubule-disruptor agents; alkylating agents, anti-metabolites; epidophyllotoxin; an antineoplastic enzyme; a topoisomerase inhibitor; procarbazine; mitoxantrone; platinum coordination complexes; biological response modifiers and growth inhibitors; hormonal/anti-hormonal therapeutic agents and haematopoietic growth factors.

Example classes of antineoplastic agents include, for example, the anthracycline family of drugs, the vinca drugs, the mitomycins, the bleomycins, the cytotoxic nucleosides, the taxanes, the epothilones, discodermolide, the pteridine family of drugs, diynenes and the podophyllotoxins. Particularly useful members of those classes include, for example, doxorubicin, carminomycin, daunorubicin, aminopterin, methotrexate, methopterin, dichloro-methotrexate, mitomycin C, porfiromycin, Herceptin®, Rituxan®, 5-fluorouracil, 6-mercaptopurine, gemcitabine, cytosine arabinoside, podophyllotoxin or podo-phyllotoxin derivatives such as colchicines, etoposide, etoposide phosphate or teniposide, melphalan, vinblastine, vincristine, leurosidine, vindesine, leurosine, paclitaxel and the like. Other useful antineoplastic agents include estramustine, cisplatin, carboplatin, cyclophosphamide, bleomycin, tamoxifen, ifosamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, pyridobenzoindole derivatives, interferons and interleukins. The preferred class of antineoplastic agents is the taxanes and the preferred antineoplastic agent is paclitaxel.

Radiation therapy, including x-rays or gamma rays which are delivered from either an externally applied beam or by implantation of tiny radioactive sources, may also be used in combination with the compounds of this invention alone to treat cancer.

### EXAMPLES

The following preparations and examples are given to enable those skilled in the art to practice and to understand more clearly the present invention. They should not be considered as limiting the scope of the invention, but merely as being illustrative and representative thereof.

### Synthetic Examples

### Example 1

### Synthesis of trans-3-(3,5-bis-trifluoromethylphenyl)-4-carboxy-2-methyl-1-oxo-1,2,3,4-tetrahydroisoquinoline

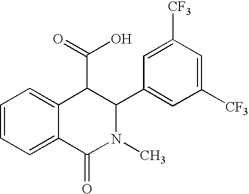

A suspension of methylamine hydrochloride (1.62 g, 24 mmol), 3,5-bis(trifluoromethyl)-benzaldehyde (5.80 g, 24 mmol), triethylamine (3.3 ml, 24 mmol), and magnesium sulfate (800 mg) in dichloromethene (30 ml) was stirred at room temperature for 8 h. Homophthalic anhydride (4.2 g, 25.9 mmol) was added and reaction mixture was heated at reflux for 15 h. After cooling down the solvent was evaporated, the residue was partitioned between ethyl acetate (300 ml) and water (100 ml). The organic phase was washed successively with 0.1 N hydrochloric acid, water, dried over sodium sulfate, filtered, and evaporated to a small volume to form a white precipitate of the title compound (8.20 g, 82.0%), which was filtered, washed with small amount of diethyl ether, and dried. The filtrate contained a mixture of cis and trans isomers, and the solid material obtained was exclusively the trans isomer, as determined by 1H-NMR and analytical HPLC. ¹H NMR (400 MHz, DMSO-d₆) δ (ppm) 7.97 (s, 1 H), 7.89 (d, J=6.8 Hz, 1H), 7.69 (s, 2 H), 7.45-7.37 (m, 2 H), 7.22 (d, J=7.2 Hz, 1H), 5.55 (s, 1 H), 4.28 (s, 1H), 3.04 (s, 3H); MS (ES) m/z 418.6 (MH⁺); MS calcd: 417.1 (M).

### Example 2

### Synthesis of trans-3-(3,5-bis-trifluoromethylphenyl)-4-carboxy-2-(2,4-dimethoxybenzyl)-1-oxo-1,2,3,4-tetrahydroisoquinoline

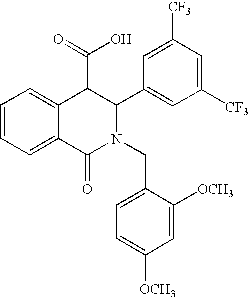 Step 1

A suspension of 3,5-bis(trifluoromethyl)benzaldehyde (15.6 g; 62.4 mmol based on reagent purity of 97%), 2,4-dimethoxybenzylamine (10.7 g; 62.4 mmol, based on reagent purity of 98%), triethylamine (18 mL; 13 g; 129 mmol) and MgSO₄ (700 mg) in CH₂Cl₂ (130 mL) was stirred for 6 h at room temperature. The reaction mixture was then treated with homophthalic anhydride (12.6 g; 78 mmol; 125 mole%/o), followed by heating at reflux for 15 h. The reaction mixture was cooled to room temperature, diluted with CH₂Cl₂ (300 mL) and the resulting suspension was filtered. The filtrate was concentrated in vacuo and the residue was partitioned between ethyl acetate (600 mL) and saturated aqueous sodium hydrogencarbonate (200 mL). The layers were separated and the organic phase was washed with saturated aqueous sodium chloride (2×150 mL), dried over MgSO₄ and concentrated in vacuo to give 39.8 g of a cis/trans mixture of 3-(3,5-bis-trifluoromethylphenyl)-4-carboxy-2-(2,4-dimethoxybenzyl)-1-oxo-1,2,3,4-tetrahydroisoquinoline as a light yellow foam. HPLC-UV and LC-MS analysis showed this material to be a 3:2 mixture of isomers.

Step 2

A 35-g sample of the above cis/trans mixture was dissolved in acetic acid (250 mL) and heated at reflux with stirring for 6 h. The reaction mixture was then allowed to cool to room temperature, frozen in a dry ice-acetone bath, and lyophilized to give 35 g of a yellow powder. Traces of triethylamine salts were removed by washing the solid with 0.65 N aqueous HCl, then drying the material overnight on a lyophilizer to remove residual water. HPLC-UV and NMR analysis indicated that this material was >95% of the desired trans-isomer. This material was used directly in the next reaction.

¹H NMR (DMSO-d6, 400 MHz) δ (ppm) 7.93-7.62 (m, 1H), 7.75 (s, 1H), 7-36-7.42 (m, 4H), 7.27 (d, J=5.0 Hz, 1H), 7.18 (t, J=3.5 Hz, 1H), 6.36 (d, J=5.5 Hz, 1H), 6.04 (s, 1H), 5.57 (s, 1H), 5.00 (d, J=8.5 Hz, 1H), 4.31 (d, J=8.5 Hz, 1H), 4.12 (s, 1H), 3.61 (s, 3H), 3.39 (s, 3H); MS (ES) m/z=554.2 (MH⁺); MS calcd: 553.1 (M).

### Example 3

### Synthesis of trans-3-(3,5-bis-trifluoromethylphenyl)-4-carboxy-1-oxo-1,2,3,4-tetrahydroisoquinoline

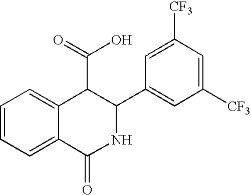

Trans-3-[3,5-bis(trifluoromethyl)phenyl]-4-carboxy-2-(2,4-dimethoxybenzyl)-1-oxo-1,2,3,4-tetrahydroisoquinoline (27.4 g; 49.5 mmol), prepared as described in Example 2 above, was added to a mixture of TFA (100 mL) and water (6 mL) and then the resulting solution was heated to reflux with stirring for a period of 2.5 h. The reaction mixture was cooled to room temperature and diluted with water (100 mL). The resulting solution was extracted with CH₂Cl₂ (5×100 mL) until HPLC-UV analysis showed that no product remained in the aqueous layer. The organic phase concentrated in vacuo to give a purple-colored solid (30 g). The residue was dissolved in water (100 mL) then 2M aqueous sodium hydroxide (50 mL) was added and the resulting suspension stirred until most of the solids had dissolved. The cloudy basic solution was washed with diethyl ether (2×100 mL), CH₂Cl₂ (200 mL) and finally acidified with 85% H₃PO₄ (100 mL) to give a precipitate which was filtered. The filtrate was extracted with CH₂Cl₂ (2×100 mL) and the precipitate was dissolved in the combined CH₂Cl₂ extracts. Residual solids from the filtration were dissolved in ethanol and this was added to the combined CH₂Cl₂ extracts. The CH₂Cl₂/ethanol mixture was filtered through a pad of celite, and the filtrate concentrated in vacuo to give 22.1 g of a brownish-yellow solid.

This solid was dissolved in a minimum amount of hot ethanol (100-150 mL) then just enough water was added until a cloudy solution was obtained. The resulting solution was allowed to cool to room temperature and filtered to remove a small amount of a solid impurity. The process was repeated once more to remove an additional amount of the solid impurity, then the filtrate was concentrated in vacuo, and the residue dissolved in an acetonitrile-water mixture (1:1; about 100 mL). This solution was frozen in a dry ice-acetone bath, then lyophilized to give 15.5 g (78%) of the title compound as an off-white powder. ¹H-NMR (DMSO-d₆, 400 MHz) δ (ppm) 8.65 (d, J=2.5 Hz, 1H), 7.97 (s, 1H), 7.91 (s, 2H), 7.87 (d, J=4.8 Hz, 1H), 7.47 (t, J=4.8 Hz, 1H), 7.39 (t, J=4.8 Hz, 1H), 7.28 (d, J=4.8 Hz, 1H), 5.36 (s, 1H), 4.45 (s, 1H); MS (ES) m/z 404.2 (MH⁺); MS calcd: 403.1 (M).

### Example 4

### Synthesis of 3-3,5-bis-trifluoromethylphenyl)-2-(2-methoxyethyl)-1-oxo-4-carboxy-1,2,3,4-tetrahydroisoquinoline

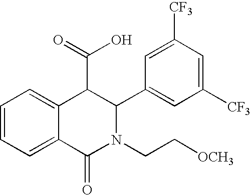

A suspension of 2-methoxyethylamine (70 μl, 0.8 mmol), 3,5-bis-(trifluoromethyl)benzaldehyde (180 mg, 0.8 mmol), triethylamine (110 μl, 0.8 mmol), and magnesium sulfate (30 mg) in dichloromethene (1 ml) was stirred at room temperature for 8 h, then homophthalic anhydride (162 mg, 1 mmol) was added and reaction mixture was heated at reflux for 15 h. After cooling, the solvent was evaporated, the residue was dissolved in 3 ml acetonitrile/water, 2:1 and purified by reversed phase HPLC; 170 mg (46.2%) of the title compound was isolated after lyophilization as a white solid. MS (ES) m/z 462.5 (MH⁺); MS calcd: 461.1 (M).

### Example 5

### Synthesis of 3-(3,5-bis-trifluoromethylphenyl)-2,3-dimethyl-1-oxo-4-carboxy-1,2,3,4-tetrahydroisoquinoline

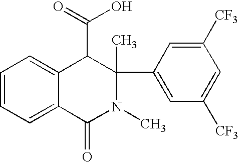

A suspension of methylamine hydrochloride (204 mg, 3 mmol), 3,5-bis(trifluoromethyl)-acetophenone (768 mg, 3 mmol), triethylamine (440 μl, 5 mmol), and magnesium sulfate (50 mg) in dichloromethane (30 ml) was heated at reflux for 15 h, then homophthalic anhydride (486 mg, 3 mmol) was added and the reaction mixture was heated at reflux for 24 h. After cooling the solvent was evaporated, the residue was dissolved in 5 ml acetonitrile/water, 2:1 and purified by reversed phase HPLC; 205 mg (15.7%) of the title compound (˜1:1 mixture of cis and trans isomers) was isolated after lyophilization as a white solid. MS (ES) m/z 432.4 (MH⁺); MS calcd: 431.1 (M).

### Example 6

### Synthesis of trans-3-(3,5-bis-trifluoromethylphenyl)-4-[(furan-2-ylmethyl)-aminocarbonyl]-2-methyl-1-oxo-1,2,3,4-tetrahydroisoquinoline

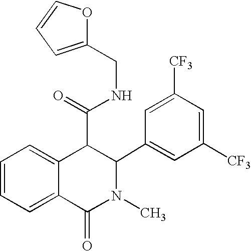

To a solution of trans-3-(3,5-bis-trifluoromethylphenyl)-4-carboxy-2-methyl-1-oxo-1,2,3,4-tetrahydroisoquinoline (7.37g, 17.6 mmol) in dichloromethane (40 ml) was added PyBOP (11.03 g, 21.2 mmol), furfurylamine (2.06 g, 21.2 mmol) and N,N-diisopropylethylamine (6.15 ml, 35.3 mmol). The reaction mixture was stirred for 15 h at room temperature, then diluted with dichloromethane (400 ml), washed with 0.1 N HCl, water, dried, concentrated, and purified by flash chromatography (ethyl acetate-hexane, 1:1). The solvent was removed by evaporation to afford 8.0 g (91.5%) of the title compound as a white solid. ¹H NMR (400 MHz, DMSO-d₆) δ (ppm) 8.68 (t, 1H), 7.99 (s, 1 H), 7.91 (m, 1H), 7.73 (s, 2 H), 7.57 (s, 1H), 7.40-7.38 (m, 2 H), 7.19-7.17 (m, 1H), 6.38 (d, J=3.6 Hz, 1H), 6.18 (s, 1H), 5.35 (s, 1 H), 4.29 (d, J=4.8 Hz, 2H), 4.08 (s, 1H), 2.97 (s, 3H); MS (ES) m/z 497.3 (MH⁺); MS calcd: 496.1 (M).

### Example 7

### Synthesis of trans-3-(3,5-bis-trifluoromethyl-phenyl)-4-[(thiazol-2-yl)aminocarbonyl]-2-methyl-1-oxo-1,2,3,4-tetrahydroisoquinoline

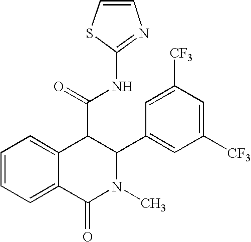

To a solution of trans-3-(3,5-bis-trifluoromethylphenyl)-4-carboxy-2-methyl-1-oxo-1,2,3,4-tetrahydroisoquinoline (1, 35 mg, 0.083 mmol) in dichloromethane (1 ml) was added PyBOP (52 mg, 0.1 mmol), 2-aminothiazole (10 mg, 0.1 mmol) and N,N-diisopropylethylamine (30 μl, 0.17 mmol). The reaction mixture was stirred for 15 h at room temperature, then solvent was evaporated and the residue was purified by reversed phase HPLC to give after lyophilization the title compound (19 mg, 46.3%) as a white solid. ¹H NMR (400 MHz, DMSO-d₆) δ (ppm) 12.67 (s, 1H), 8.02 (s, 1 H), 7.97-7.95 (m, 1H), 7.81 (s, 2 H), 7.51 (d, J=3.6 Hz, 1H), 7.44-7.41 (m, 2H), 7.33-7.31 (m, 1H), 7.24 (d, J=4.0 Hz, 1H), 5.51 (s, 1 H), 4.37 (d, J=1.2 Hz, 1H), 3.02 (s, 3H); MS (ES) m/z 500.2 (MH⁺); MS calcd: 499.1 (M).

### Example 8

### Synthesis of trans-3-(3,5-bis-trifluoromethylphenyl)-4-[(2-methoxyethyl)-aminocarbonyl]2-(2-methoxyethyl)-1-oxo-1,2,3,4-tetrahydroisoquinoline

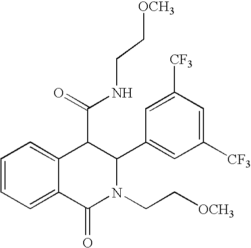

To a solution of 3-(3,5-bis-trifluoromethyl-phenyl)-4-carboxy-2-(2-methoxyethyl)-1-oxo-1,2,3,4-tetrahydroisoquinoline (2, 33 mg, 0.072 mmol) in DMF (1 ml) was added PyBOP (45 mg, 0.086 mmol), 2-methoxyethylamine (10 μl, 0.1 mmol) and N,N-diisopropylethylamine (30 μl, 0.17 mmol). The reaction mixture was stirred for 15 h at room temperature, then solvent was evaporated in high vacuum and the residue was treated with acetonitrile/water, 1: 1 to form a white precipitate, which was filtered, washed with water and dried to give the title compound (10 mg, 27.0%) as a white solid. ¹H NMR (400 MHz, DMSO-d₆) δ (ppm) 8.34 (t, J=5.2 Hz, 1H), 7.94 (s, 1 H), 7.90-7.87 (m, 1H), 7.76 (s, 2 H), 7.37-7.35 (m, 2H), 7.10-7.08 (m, 1H), 5.41 (s, 1 H), 4.04 (d, J=1.2 Hz, 1H), 3.71-3.65 (m, 1H), 3.54-3.48 (m, 1H), 3.40-3.22 (m, 9H), 3.00 (s, 3H); MS (ES) m/z 519.4 (MH⁺); MS calcd: 518.2 (M).

### Example 9

### Synthesis of trans-3-(3,5-bis-trifluoromethyl-phenyl)-4-[(morpholin-4-yl)-aminocarbonyl]-1-oxo-1,2,3,4-tetrahydroisoquinoline

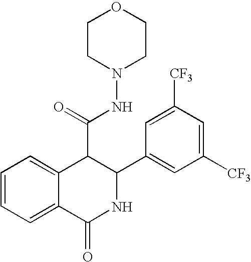

To a solution of trans-3-(3,5-bis-trifluoromethyl-phenyl)-4-carboxy-1-oxo-1,2,3,4-tetrahydroisoquinoline (30 mg, 0.075 mmol) in dichloromethane (1 ml) was added PyBOP (52 mg, 0.1 mmol), 4-aminomorpholine (10 mg, 0.1 mmol) and N,N-diisopropylethylamine (30 μl, 0.17 mmol). The reaction mixture was stirred for 15 h at room temperature, then solvent was evaporated and the residue was purified by reversed phase HPLC to give after lyophilization the title compound (30 mg, 83.2%, 1:1 mixture of cis and trans isomers) as a white solid; MS (ES) m/z 488.4 (MH⁺); MS calcd: 487.1 (M).

### Example 10

### Synthesis of 3-(3,5-bis-trifluoromethylphenyl)-4-[(furan-2-ylmethyl)-aminocarbonyl]-2,3-dimethyl-1-oxo-1,2,3,4-tetrahydroisoquinoline

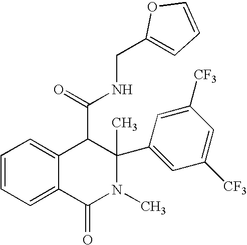

To a solution of 3-(3,5-bis-trifluoromethyl-phenyl)-4-carboxy-2,3-dimethyl-1-oxo-1,2,3,4-tetrahydroisoquinoline (3, 35 mg, 0.081 mmol) in dichloromethane (1 ml) was added PyBOP (52 mg, 0.1 mmol), furfurylamine (10 μl, 0.1 mmol) and N)N-diisopropylethylamine (30 μl, 0.17 mmol). The reaction mixture was stirred for 15 h at room temperature, then the solvent was evaporated and the residue was treated with acetonitrile/water, 1:1 to form a white precipitate, which was filtered, washed with water and dried to give the title compound as 1:1 mixture of cis and trails isomers (36 mg, 87.8%) as a white solid; MS (ES) m/z 511.1 (MH⁺); MS calcd: 510.1 (M).

### Example 11

### Synthesis of trans-3-(3,5-bis-trifluoromethylphenyl)-4-[(3-hydroxypropyl)aminocarbonyl]-2-methyl-1-oxo-1,2,3,4-tetrahydroisoquinoline

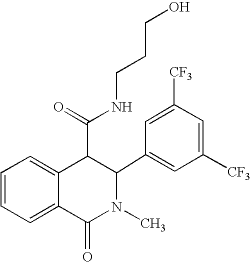

To a solution of trans-3-(3,5-bis-trifluoromethylphenyl)-4-carboxy-2-dimethyl-1-oxo-1,2,3,4-tetrahydroisoquinoline (3, 35 mg, 0.081 mmol) in dichloromethane (1 ml) was added PyBOP (52 mg, 0.1 mmol), propanolamine (10 μl, 0.1 mmol) and N,N-diisopropylethylamine (30 μl, 0.17 mmol). The reaction mixture was stirred for 15 h at room temperature, then the solvent was evaporated and the residue was treated with the mixture of acetonitrile/water, 1:1 to form a white precipitate, which was filtered, washed with water and dried to give the title compound (38 mg, 97.2%) as a white solid. ¹H NMR (400 MHz, DMSO-d₆) δ (ppm) 8.11 (t, J=5.6 Hz, 1H), 7.99 (s, 1 H), 7.92-7.89 (m, 1H), 7.72 (s, 2 H), 7.40-7-36 (m, 2H), 7.18-7.16 (m, 1H), 5.32 (d, J=1.2 Hz, 1H), 4.13 (t, J=4.8 Hz, 1H), 4.00 (d, J=2.0 Hz, 1H1), 3.36 (q, J=6.0 Hz, J=11.6 Hz, 2H), 3.15-3.09 ( m, 2H), 2.97 (s, 3H), 1.57-1.52 (m, 2H) ; MS (ES) m/z 475.1 (MH⁺); MS calcd: 474.1(M).

### Example 12

### Synthesis of trans-3-(3,5-bis-trifluoromethyl-phenyl)-4-[(5,6-dimethyl-[1,2,4]triazin-3-yl)aminocarbonyl]-2-methyl-1-oxo-1,2,3,4-tetrahydroisoquinoline

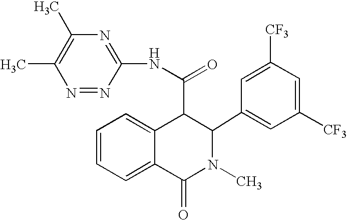

To a solution of trans-3-(3,5-bis-trifluoromethyl-phenyl)-4-carboxy-2-methyl-1-oxo-1,2,3,4-tetrahydroisoquinoline (500 mg, 1.2 mmol) in dimethylformamide (5 ml) was added HATU (380 mg, 1.44 mmol), 3-amino-5,6-dimethyl-1,2,4-triazine (445 mg, 3.6 mmol) and N,N-diisopropylethylamine (430 μl, 2.4 mmol). The reaction mixture was stirred for 36 h at room temperature, then partitioned between ethyl acetate and water (20 ml/20 ml). The organic layer was washed with saturated sodium bicarbonate solution, 0.5 N aqueous HCl, water, brine, dried over magnesium sulfate; then solvent was evaporated and the residue was purified by flash chromatography on silica gel (using ethyl acetate as eluent). The solvent was removed by evaporation to give the title compound (160 mg, 25.5%) as a white solid. ¹H NMR (400 MHz, DMSO-d₆) δ (ppm) 11.38 (t, 1H), 8.01 (s, 1 H), 7.96-7.94 (m, 1H), 7.83 (s, 2 H), 7.42-7.40 (m, 2H), 7.33-7.31 (m, 1H), 5.52 (s, 1 H), 4.46 (s, 1H), 3.02 (s, 3H), 2.56 (s, 3H), 2.45 (s, 3H); MS (ES). m/z 524.2 (MH⁺); MS calcd: 523.1 (M).

### Example 13

### Synthesis of trans-3-(3,5-bis-trifluoromethyl-phenyl)-4-[(4-methyl-pyrimidin-2-yl)aminocarbonyl]-2-methyl-1-oxo-1,2,3,4-tetrahydroisoquinoline

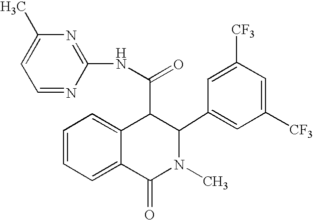

To a solution of trans-3-(3,5-bis-trifluoromethyl-phenyl)-4-carboxy-2-methyl-1-oxo-1,2,3,4-tetrahydroisoquinoline (1, 500 mg, 1.2 mmol) in dimethylformamide (5 ml) was added HATU (380 mg, 1.44 mmol), 2-amino4-methylpyrimidine (390 mg, 3.6 mmol) and N,N-diisopropylethylamine (430 μl, 2.4 mmol). The reaction mixture was stirred for 36 h at room temperature, then partitioned between ethyl acetate and water (20 ml/20 ml). The organic layer was washed with saturated sodium bicarbonate solution, 0.5 N aqueous HCl, water, brine, and dried over magnesium sulfate. The solvent was evaporated and the residue was purified by flash chromatography on silica gel (using ethyl acetate as eluent). The solvent was removed by evaporation to give the title compound (80 mg, 12.7%) as a white solid. ¹H NMR (400 MHz, DMSO-d₆) δ (ppm) 11.05 (s, 1H), 8.47 (d, J=5.2 Hz, 1H), 8.00 (s, 1H), 7.95-7.93 (m, 1H), 7.81 (s, 2 H), 7.41-7.39 (m, 2H), 7.31-7.29 (m, 1H), 7.08 (d, J=5.2 Hz, 1H), 5.48 (s, 1 H), 4.46 (s, 1H), 3.02 (s, 3H), 2.41 (s, 3H); MS (ES) m/z 509.2 (MH⁺); MS calcd: 508.1 (M).

### Example 14

### Synthesis of trans-3-(3,5-bis-trifluoromethyl-phenyl)-4-[(2,4-dioxo-1,2,3,4-tetrahydro-pyrimidin-5-yl)aminocarbonyl]-2-methyl-1-oxo-1,2,3,4-tetrahydroisoquinoline

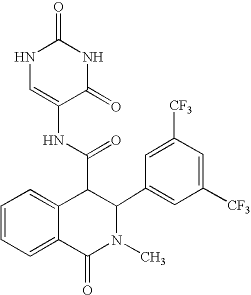

A solution of trans-3-(3,5-bis-trifluoromethylphenyl)-4-carboxy-2-methyl-1-oxo-1,2,3,4-tetrahydroisoquinoline (1, 85 mg, 0.2 mmol) in thionyl chloride (1 ml) was heated for 1 h at 60° C., then the solvent was evaporated to give the corresponding acyl chloride as a yellowish solid, which was dried under reduced pressure. A solution of acyl chloride in dimethylacetamide (1 ml) was treated with 5-aminouracil (32 mg, 0.25 mmol) and N,N-diisopropylethylamine (70 μl, 0.4 mmol) and the reaction mixture was allowed to stirr for 15 h at room temperature. The reaction was purified directly by reversed phase HPLC to give after lyophillzation the title compound (36 mg, 34.2%) as a white solid. ¹H NMR (400 MHz, DMSO-d₆) δ (ppm) 11.50 (d, J=2.0 Hz, 1 H), 10.66 (dd, J=2.0 Hz, J=6.0 Hz, 1 H), 9.67 (s, 1H), 8.07 (d, J=5.6 Hz, 1 H), 7.99 (s, 1H), 7.92-7.90 (m, 1 H), 7.75 (s, 2H), 7.40-7.38 (m, 2H), 7.24 (m, 1H), 5.42 (s, 1 H), 4.60 (d, J=1.6 Hz, 1H)m 3.00 (s, 3H); MS (ES) m/z 527.2 (MH⁺); MS calcd: 526.1 (M).

### Example 15

### Synthesis of trans-3-(3,5-bis-trifluoromethyl-phenyl)-4-[(2-furoyl)aminocarbonyl]-2-methyl-1-oxo-1,2,3,4-tetrahydroisoquinoline-

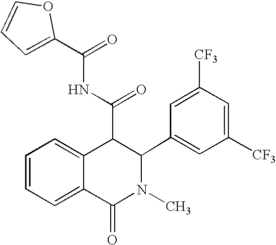

To a solution of 2-furamide (170 mg, 1.53 mmol) in anhydrous THE (5 ml), at 0° C. was added sodium hydride ( 60% in mineral oil, 90 mg, 2.25 mmol). The reaction mixture was stirred for 15 min at 0° C., then treated with 3-(3,5-bis-trifluoromethyl-phenyl)-2-methyl-1-oxo-1,2,3,4-tetrahydroisoquinoline-4-carbonyl chloride (660 mg, 1.52 mmol; prepared as described in example 14). After 15 h at room temperature the reaction mixture was carefully quenched with water and extracted with ethyl acetate (50 ml). The organic layer was washed with water, 0.1 N HCl, brine, dried, and concentrated under reduced pressure. The title compound (82 mg, 10.7%) crystallized from ethyl acetate solution as a white solid. ¹H NMR (400 MHz, DMSO) δ (ppm) 11.28 (s, 1H), 8.10 (d, J=1.6 Hz, 1 H), 8.05 (s, 1H), 7.99-7.97 (m, 1 H), 7.84 (s, 2H), 7.68 (d, J=3.6 Hz, 1H), 7.46-7.44 (m, 2H), 7.35-7.33 (m, 1H), 6.80-6.79 (m, 1H), 5.61 (s, 1 H), 4.96 (s, 1H), 3.05 (s, 3H); MS (ES) m/z 511.1 (MH⁺); MS calcd: 510.1 (M).

### Biological Examples

### Example 1

### Identification of Caspase Cascade Activators and Inducers of Apoptosis in Solid Tumor Cells

Human breast cancer cell lines T47D and ZR-75-1 were grown according to media component mixtures designated by American Type Culture Collection +10% fetal calf sera (FCS) (Invitrogen Corporation) in a 5% CO₂-95% humidity incubator as 37° C. The T-47 and ZR-75-1 cells were maintained at a cell density between 30 and 80% confluency at a cell density of 0.1 to 0.6×10⁶ cells/mnL.

Cells were harvested at 600× g and resuspended at 0.65×10⁶ cells/mL into appropriate media +10% FCS. An aliquot of 45 μL of cells was added to a well of a 96-well microtiter plate containing 5 μL of a 10% DMSO in RPMI-1640 media solution containing 1.6 to 100 μM of test compound (0.16 to 10 μM final). An aliquot of 45 μL of cells was added to a well of a 96-well microtiter plate containing 5 μL of a 10% DMSO in RPMI-1640 media solution without test compound as the control sample. The samples were mixed by agitation and then incubated at 37° C. for 24 h in a 5% CO₂-95% humidity incubator. After incubation, the samples were removed from the incubator and 50 μL of a solution containing 20 μL of N-(Ac-DEVD)-N′-ethoxycarbonyl-R110 fluorogenic substrate (Cytovia, Inc.; WO99/18856), 20% sucrose (Sigma), 20 mM dithiothreitol (DTT) (Sigma), 200 mM NaCl (Sigma), 40 mM Na piperazine-N,N′-bis[2-ethanesulfonic acid] (PIPES) buffer pH 7.2 (Sigma), and 500 μg/mL lysolecithin (Calbiochem) was added. The samples were mixed by agitation and incubated at room temperature. Using a fluorescent plate reader (Model 1420 Wallac Instruments), an initial reading (T=0) was made approximately 1-2 minutes after addition of the substrate solution, employing excitation at 485 nm and emission at 530 nm, to determine the background fluorescence of the control sample. After the 3 hour incubation, the samples were read for fluorescence as above (T=3 h).

Calculation:

The Relative Fluorescence Unit (RFU) values were used to calculate the sample readings as follows:
RFU_{(T=3h)}−Control RFU_{(T=0)}=Net RFU_{(T=3h)}

The level of caspase cascade activation was determined by the ratio of the net RFU value for the test compound to that of the control samples. The EC₅₀ (nM) was determined by a sigmoidal dose-response calculation (Prism 2.0, GraphPad Software, Inc.). The compounds of the invention were determined to have caspase cascade activating effects by proceeding as in Example 1.

### Example 2

### Identification of Antineoplastic Activity in Cell Proliferation

T-47D and ZR-75-1 cells are grown and harvested by proceeding as in Example 1.

An aliquot of 90 μL of cells (2.2×10⁴ cells/mL) is added to a well of a 96-well microtiter plate containing 10 μL of a 10% DMSO in PRMI-1640 media solution containing 1 mM to 100 μM of test compound. An aliquot of 90 μL of cells is added to a well of a 96-well microtiter plate containing 10 μL of a 10% DMSO in RPMI-1640 media solution without test compound as the control sample for maximal cell proliferation (Aₘₐₓ). The samples are mixed by agitation and then incubated at 37° C. for 48 h in a 5% CO₂-95% humidity incubator. After incubation, the samples are removed from the incubator and 20 μL of CellTiter 96 Aqueous One Solution Cell Proliferation® reagent (Promega) is added. The samples are mixed by agitation and incubated at 37° C. for 24, h in a 5% CO₂-95% humidity incubator. Using an absorbance plate reader (Model 1420 Wallac Instruments), an initial reading (T=0) is made approximately 1-2 minutes after addition of the solution, employing absorbance at 490 nm, to determine any background absorbance of the test compound. After the 2-4 h incubation, the samples are read for absorbance as above (Aₜₑₛₜ).

Baseline for the dose producing 50% inhibition of cell proliferation (GI₅₀) of initial cell numbers is determined by adding an aliquot of 90 μL of cells or 90 μL of media, respectively, to wells of a 96-well microtiter plate containing 10 μL of a 10% DMSO in RPMI-1640 media solution. The samples are mixed by agitation and then incubated at 37° C. for 0.5 h in a 5% CO₂-95% humidity incubator. After incubation, the samples are removed from the incubator and 20 μL of CellTiter 96 Aqueous One Solution Cell Proliferation® reagent (Promega) is added. The samples are mixed by agitation and incubated at 37° C. for 24 h in a 5% CO₂-95% humidity incubator. Absorbance is read as above, (A_{T=0}) defining absorbance for initial cell number used as baseline GI₅₀ determinations.
Calculation: GI₅₀(nM)=100×[Aₜₑₛₜ−A_{T=0}/(Aₘₐₓ−A_{T=0})].

### Example 3

### Nuclear Fragmentation in T47D Cells

T47D cells are grown and harvested by proceeding as in Example 1 and treated with test compound followed by staining of the cell nuclei with Syto 16, a fluorescent DNA dye which stains nuclei. Shrunken and fragmented nuclei are hallmarks of caspase-mediated apoptosis. T47D cells treated with test compound for 48 h exhibit shrunken and fragmented nuclei.

### Example 4

### Mitotic Arrest in Jurkat Cells

Jurkat cells are incubated with a range of concentrations of test compounds (0.02 μM to 5 μM) for 6 h under normal growth conditions. Control cultures are treated with DMSO vehicle. The cells are then treated for 20 minutes with 800 nM Syto 16. Cytospin preparation is then prepared and the samples were viewed by fluorescent microscopy using a fluorescein filter set. For each concentration of test compound, the number of mitotic figures are counted and expressed as a percentage of the total number of cells. Three fields from each condition are evaluated and the mean and SEM were calculated and plotted as a function of drug concentration.

### Example 5

### Cell Cycle Arrest in Solid Tumor Cell Lines

T47D cells are grown and harvested by proceeding as in Example 1. 10⁶ Cells are treated with test compound for 48 h at 37° C. As a control, cells are also incubated with DMSO. Cells were harvested at 1200 rpm and washed twice with 5 mM EDTA/PBS. Cells are then resuspended in 300 μL of EDTA/PBS and 700 mL of 100% ethanol, vortexed and incubated at room temperature for 1 hour. Samples are spun down at 12000 rpm for 5 minutes and the supernatant is removed. A solution containing 100 μg/mL of propidium iodide and 1 mg/mL of RNAse A (fresh) is added to the samples and the samples are incubated for 1 hour at room temperature. Samples are then transferred to 12×75 mm polystyrene tubes and analyzed on a flow cytometer. All flow cytometry analyses are performed on FACScalibur (Becton Dickison) using Cell Quest analysis software.

### Pharmaceutical Composition Examples

The following are representative pharmaceutical formulations containing a compound of Formula I or Ia.

### Tablet Formulation

The following ingredients are mixed intimately and pressed into single scored tablets.

| Ingredient | Quantity per tablet, mg |
|---|---|
| compound of this invention | 400 |
| cornstarch | 50 |
| croscarmellose sodium | 25 |
| lactosemagnesium stearate | 120 |

### Capsule Formulation

The following ingredients are mixed intimately and loaded into a hard-shell gelatin capsule.

| Ingredient | Quantity per capsule, mg |
|---|---|
| compound of this | 200 |
| invention | |
| lactose, spray-dried | 148 |
| magnesium stearate | 2 |

### Suspension Formulation

The following ingredients are mixed to form a suspension for oral administration.

| Ingredient | Amount | |
|---|---|---|
| compound of this invention | 1.0 | g |
| fumaric acid | 0.5 | g |
| sodium chloride | 2.0 | g |
| methyl paraben | 0.15 | g |
| propyl paraben | 0.05 | g |
| granulated sugar | 25.5 | g |
| sorbitol (70% solution) | 12.85 | g |
| Veegum K (Vanderbilt Co.) | 1.0 | g |
| flavoring | 0.035 | ml |
| colorings | 0.5 | mg |
| distilled water | q.s. to 100 | ml |

### Injectable Formulation

The following ingredients are mixed to form an injectable formulation.

| Ingredient | Amount |
|---|---|
| compound of this invention | 1.2 g |
| sodium acetate buffer solution | 0.4 M, 2.0 ml |
| HCl (1 N) or NaOH (1 M) | q.s. to suitable pH |
| water (distilled, sterile) | q.s. to 20 ml |

All of the above ingredients, except water, are combined and heated to 60-70.degree. C. with stirring. A sufficient quantity of water at 60.degree. C. is then added with vigorous stirring to emulsify the ingredients, and water then added q.s. to 100 g.

### Suppository Formulation

A suppository of total weight 2.5 g is prepared by mixing the compound of the invention with Witepsol.RTM. H-15 (triglycerides of saturated vegetable fatty acid; Riches-Nelson, Inc., New York), and has the following composition:

| Ingredient | Amount |
|---|---|
| compound of this invention | 500 mg |
| Witepsol ® H-15 | balance |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

1. A compound of Formula I: 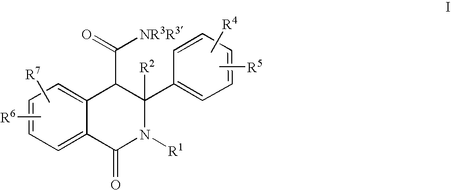
wherein:
R¹ is hydrogen, alkyl, cycloalkyl, cycloalkylalkyl, hydroxy, alkoxy, alkoxyalkyl, alkoxycarbonylalkyl, hydroxyalkyl, aryl, heteroaryl, aralkyl, heteroaralkyl, heterocycloalkylalkyl, or -alkylene-CONR⁸R⁹ where R⁸ is hydrogen, alkyl or alkoxyalkyl, and R⁹ is alkyl, optionally substituted aryl, optionally substituted aralkyl, alkoxyalkyl, optionally substituted heteroaryl, optionally substituted heteroaralkyl, heterocycloalkylalkyl, or saturated or unsaturated heterocycloaminoalkyl, or R⁸ and R⁹ together with the nitrogen atom to which they are attached form heterocycloamino;
R² is hydrogen or alkyl;
R³ is alkyl, alkoxy, hydroxy, haloalkyl, alkylthioalkyl, cycloalkyl, cycloalkylalkyl, hydroxyalkyl, alkoxyalkyl, alkoxycarbonylalkyl, carboxyalkyl, substituted carboxyalkyl, guanidino, heterocycloamino, aminoalkyl, substituted aminoalkyl, heterocycloaminoalkyl, alkylsulfonylalkyl, alkylsulfinylalkyl, heterocycloalkyl, optionally substituted aryl, optionally substituted heteroaryl, optionally substituted aralkyl, optionally substituted heteroaralkyl, aralkenyl, aryloxyalkyl, heteroaryloxyalkyl, -[(alkylene)-O]ₘ-(alkylene)-NH₂ (where m is 1, 2, or 3), heterocycloalkylalkyl, —C(O)R¹² where R¹² is optionally substituted heteroaryl, or -(alkylene)-NR¹⁰R¹⁰ where R¹⁰ and R¹¹ are independently selected from hydrogen, alkyl, optionally substituted aryl, optionally substituted heteroaryl, optionally substituted aralkyl, optionally substituted heteroaralkyl, or R¹⁰ and R¹¹ together with the nitrogen atom to which they are attached form saturated or unsaturated heterocycloamino;
R^{3′} is hydrogen or alkyl, or R^{3′} together with R³ and the nitrogen to which they are attached form heteroaryl or heterocycloamino;
R⁴ and R⁵ are independently of each other hydrogen, alkyl, halo, trifluoromethylthio, haloalkoxy, or haloalkyl; and
R⁶ and R⁷ are independently of each other hydrogen, alkyl, alkoxy, hydroxy, halo, haloalkyl, amino, alkylamino, dialkylamino, or acylamino; or
a pharmaceutically acceptable salt thereof;
provided that:
a) when R¹ is methyl, R², R^{3′}, R⁴, R⁵, R⁶ and R⁷ are hydrogen, then R³ is not —CH₂CO₂CH₃;
b) when R¹ is phenyl and R², R⁴, R⁵, R⁶, and R⁷ are hydrogen, then R³ and R^{3′} together with the nitrogen to which they are attached do not form pyrrolidinyl, piperidinyl, or morpholin-4-yl;
c) when R¹ is -alkylene-CONR⁸R⁹ and R² and R⁸ are hydrogen, then R^{3′} is hydrogen and R³ is aryloxyalkyl or substituted heterocycloalkyl (provided that substituted heterocycloamino is not substituted with alkoxyalkyl, alkyl, or hydroxyalkyl); or R³ and R^{3′} together with the nitrogen to which they are attached form substituted heterocycloamino (provided that the heterocycloamino is not substituted with hydroxy, hydroxyalkyl, or alkyl); or R⁹ is optionally substituted phenylalkyl.

2. The compound of claim 1 wherein R¹ is hydrogen or alkyl.

3. The compound of claim 1 wherein R¹ is -alkylene-CONR⁸R⁹, where R⁸ and R⁹ together with the nitrogen atom to which they are attached form heterocycloamino.

4. The compound of claim 3 wherein R¹ is 2-(piperidin-1-ylcarbonyl)ethyl, 2-(4-hydroxypiperidin-1-ylcarbonyl)ethyl, 2-(morpholin-4-ylcarbonyl)ethyl, 2-(4-acetylpiperazin-1-ylcarbonyl)ethyl, 2-(4-methylpiperidin-1-ylcarbonyl)ethyl, 2-(thiomorpholin-4-ylcarbonyl)ethyl, or 2-(4-formylpiperazin-1-ylcarbonyl)ethyl.

5. The compound of claim 2 wherein R⁴ and R⁵ are trifluoromethyl and are located at the 3- and 5-position of the phenyl ring; and
R⁶ and R⁷ are hydrogen.

6. The compound of claim 3 wherein R⁴ and R⁵ are trifluoromethyl and are located at the 3- and 5-position of the phenyl ring; and
R⁶ and R⁷ are hydrogen.

7. The compound of claim 5 wherein R² is hydrogen;
R³ is alkyl, alkoxy, hydroxy, haloalkyl, alkylthioalkyl, cycloalkyl, cycloalkylalkyl, hydroxyalkyl, alkoxyalkyl, alkoxycarbonylalkyl, carboxyalkyl, substituted carboxyalkyl, guanidino, heterocycloamino, aminoalkyl, substituted aminoalkyl, heterocycloaminoalkyl, alkylsulfonylalkyl, alkylsulfinylalkyl, heterocycloalkyl, optionally substituted aryl, optionally substituted heteroaryl, optionally substituted aralkyl, optionally substituted heteroaralkyl, aralkenyl, aryloxyalkyl, heteroaryloxyalkyl, -[(alkylene)-O]ₘ-(alkylene)-NH₂ (where m is 1, 2, or 3), heterocycloalkylalkyl, —C(O)R¹² where R¹² is optionally substituted heteroaryl, or -(alkylene)-NR¹⁰R¹¹ where R¹⁰ and R¹¹ are independently selected from hydrogen, alkyl, optionally substituted aryl, optionally substituted heteroaryl, optionally substituted aralkyl, optionally substituted heteroaralkyl, or R¹⁰ and R¹¹ together with the nitrogen atom to which they are attached form saturated or unsaturated heterocycloamino; and
R^{3′} is hydrogen or alkyl; or
R^{3′} together with R³ and the nitrogen to which they are attached form heteroaryl or heterocycloamino.

8. The compound of claim 5 wherein R² is methyl;
R³ is alkyl, alkoxy, hydroxy, haloalkyl, alkylthioalkyl, cycloalkyl, cycloalkylalkyl, hydroxyalkyl, alkoxyalkyl, alkoxycarbonylalkyl, carboxyalkyl, substituted carboxyalkyl, guanidino, heterocycloamino, aminoalkyl, substituted aminoalkyl, heterocycloaminoalkyl, alkylsulfonylalkyl, alkylsulfinylalkyl, heterocycloalkyl, optionally substituted aryl, optionally substituted heteroaryl, optionally substituted arallyl, optionally substituted heteroaralkyl, aralkenyl, aryloxyalkyl, heteroaryloxyalkyl, -[(alkylene)-O]ₘ-(alkylene)-NH₂ (where m is 1, 2, or 3), heterocycloalkylalkyl, —C(O)R¹² where R¹² is optionally substituted heteroaryl, or -(alkylene)-NR¹⁰R¹¹ where R¹⁰ and R¹¹ are independently selected from hydrogen, alkyl, optionally substituted aryl, optionally substituted heteroaryl, optionally substituted aralkyl, optionally substituted heteroaralkyl, or R¹⁰ and R¹¹ together with the nitrogen atom to which they are attached form saturated or unsaturated heterocycloamino; and
R^{3′} is hydrogen or alkyl; or
R^{3′} together with R³ and the nitrogen to which they are attached form heteroaryl or heterocycloamino.

9. The compound of claim 6 wherein R² is hydrogen;
R³ is alkyl, alkoxy, hydroxy, haloalkyl, alkylthioalkyl, cycloalkyl, cycloalkylalkyl, hydroxyalkyl, alkoxyalkyl, alkoxycarbonylalkyl, carboxyalkyl, substituted carboxyalkyl, guanidino, heterocycloamino, aminoalkyl, substituted aminoalkyl, heterocycloaminoalkyl, alkylsulfonylalkyl, alkylsulfinylalkyl, heterocycloalkyl, optionally substituted aryl, optionally substituted heteroaryl, optionally substituted aralkyl, optionally substituted heteroaralkyl, aralkenyl, aryloxyalkyl, heteroaryloxyalkyl, -[(alkylene)-O]ₘ-(alkylene)-NH₂ (where m is 1, 2, or 3), heterocycloalkylalkyl, —C(O)R¹² where R¹² is optionally substituted heteroaryl, or -(alkylene)-NR¹⁰R¹¹ where R¹⁰ and R¹¹ are independently selected from hydrogen, alkyl, optionally substituted aryl, optionally substituted heteroaryl, optionally substituted aralkyl, optionally substituted heteroaralkyl, or R¹⁰ and R¹¹ together with the nitrogen atom to which they are attached form saturated or unsaturated heterocycloamino; and
R^{3′} is hydrogen or alkyl; or
R^{3′} together with R³ and the nitrogen to which they are attached form heteroaryl or heterocycloamino.

10. The compound of claim 7 wherein R³ is 2-hydroxypyrid-6-yl, 2-chloropyrid-3-yl, 2-thio-[1,3,4]-thiadiazol-2-yl, 5,8-diphenyl-[1,2,4]triazocin-3-yl, 6-ethoxy-benzothiazol-2-yl, 6-fluoro-benzothiazol-2-yl, 3,5-dimethylisoxazol-4-yl, 5-methylisoxazol-3-ylmethyl, pyrimidin-2-yl, 3-methylpyrid-2-yl, 4-methylpyrid-2-yl, 5-methylpyrid-2-yl, 6-methylpyrid-2-yl, 4,6-dimethylpyrid-2-yl, 3-methylpyrid-4-yl, 2-methylpyrid-4-yl, 1,3-dimethylpyrazol-5-yl, 5-methylpyrazol-3-yl, 4-methylpyrimidin-2-yl, 4,6-dimethylpyrimidin-2-yl, 2,4-dimethylpyrimidin-6-yl, pyrazin-2-yl, pyrid-4-yl, pyrid-2-yl, pyrid-3-yl, pyrazol-3-yl, furan-2-ylmethyl, furan-2-ylcarbonyl, 5,6-dimethyl-[1,2,4]-triazin-3-yl, pyrimidin-4-yl, [1,3,4]-thiadiazol-2-yl, thiazol-2-yl, isoxazol-3-yl, cyclopentyl, 1H-pyrimidin-2,4-dione-5-yl, 2-methoxyethyl, cyclobutyl, cyclopropylmethyl, 3-hydroxyprop-2-yl, cyclohexylmethyl, pyrid-2-ylmethyl, pyrid-3-ylmethyl, pyrid-4-ylmethyl, pyrid-4-ylethyl, imidazol-4-ylethyl, thiophen-2-ylmethyl, cyclopentylmethyl, 2-hydroxypropyl, 3-hydroxypropyl, 2,3-dihydroxypropyl, 2-hydroxyethyl, 2-ethoxyethyl, 5-methylfuran-2-ylmethyl, cyclopropyl, cyclohexyl, 3-methoxypropyl, 1-hydroxy-4-methylpent-2-yl, 1-(furan-2-yl)ethyl, 5-(dimethylaminomethyl)furan-2-ylmethyl, 5-bromofuran-2-ylmethyl, 5-chlorofuran-2-methyl, 1,3-dihydroxyprop-2-yl, 1,3-dihydroxy-2-methylprop-2-yl, 3-hydroxy-2-methylprop-2-yl, 3-methoxyprop-2-yl, 1-tert-butyl-2-hydroxyethyl, 1-hydroxy-3-methylpent-2-yl, 1,3-dihydroxy-2-hydroxymethylprop-2-yl, 1,3-dihydroxybut-2-yl, 1,2-dimethylpyrrol-5-ylmethyl, 1-methylpyrrol-2-ylmethyl, imidazol-1-ylpropyl, furan-3-ylmethyl, 2,5-dimethylfuran-3-ylmethyl, 3-(methoxycarbonyl)furan-2-ylmethyl, 6-hydroxyhexyl, N-benzylpiperidin-4-yl, N-benzylpyrrolidin-3-yl, 2-phenyloxyethyl, benzyl, morpholin-4-yl, 2-(morpholin-4-yl)ethyl, 4,5-dihydrothiazol-2-yl, piperidin-4-yl, piperidin-4-ylmethyl, 2-methylpropyl, tert-butyl, methyl, tetrahydrofuran-2-ylmethyl, hydroxy, methoxy, ethyl, propyl, 2-fluoroethyl, 2,2,2-trifluoroethyl, 2,2,3,3,3-pentafluoropropyl, 2-methylthioethyl, —(CH₂)₂O(CH₂)₂O(CH₂)₂NH₂, 1-carboxy-3-methylbut-1-yl, 2-carboxyethyl, 3-aminocarbonyl-1-carboxypropyl, 1-carboxy-2-methylbutyl, carboxymethyl, 1-carboxy-2-methylpropyl, 2-phenyl-1-carboxyethyl, 2,3-dimethoxyphenylmethyl, 3,5-dimethoxyphenylmethyl, 3,4-difluorophenylmethyl, 2,4-difluorophenylmethyl, 4-fluorophenylmethyl, 3-difluoromethoxyphenylmethyl, 2,6-dimethoxyphenylmethyl, 2-(methylsulfinyl)ethyl, 2-(methylsulfonyl)ethyl, 2-hydroxyphenyl, 4-chloro-2-hydroxyphenyl, 2-amino-4-oxo-3H-pyrimidin-6-yl, 2-cyanophenyl, 5-amino-1-carboxypentyl, 5-amino-1-aminocarbonylpentyl, 2-(4-methoxyphenyl)ethyl, or guanidino; and
R^{3′} is hydrogen or methyl.

11. The compound of claim 8 wherein R³ is 2-hydroxypyrid-6-yl, 2-chloropyrid-3-yl, 2-thio-[1,3,4]-thiadiazol-2-yl, 5,8-diphenyl-[1,2,4]triazocin-3-yl, 6-ethoxy-benzothiazol-2-yl, 6-fluoro-benzothiazol-2-yl, 3,5-dimethylisoxazol-4-yl, 5-methylisoxazol-3-ylmethyl, pyrimidin-2-yl, 3-methylpyrid-2-yl, 4-methylpyrid-2-yl, 5-methylpyrid-2-yl, 6-methylpyrid-2-yl, 4,6-dimethylpyrid-2-yl, 3-methylpyrid-4-yl, 2-methylpyrid-4-yl, 1,3-dimethylpyrazol-5-yl, 5-methylpyrazol-3-yl, 4-methylpyrimidin-2-yl, 4,6-dimethylpyrimidin-2-yl, 2,4-dimethylpyrimidin-6-yl, pyrazin-2-yl, pyrid-4-yl, pyrid-2-yl, pyrid-3-yl, pyrazol-3-yl, furan-2-ylmethyl, furan-2-ylcarbonyl, 5,6-dimethyl-[1,2,4]-triazin-3-yl, pyrimidin-4-yl, [1,3,4]-thiadiazol-2-yl, thiazol-2-yl, isoxazol-3-yl, cyclopentyl, 1H-pyrimidin-2,4-dione-5-yl, 2-methoxyethyl, cyclobutyl, cyclopropylmethyl, 3-hydroxyprop-2-yl, cyclohexylmethyl, pyrid-2-ylmethyl, pyrid-3-ylmethyl, pyrid-4-ylmethyl, pyrid-4-ylethyl, imidazol-4-ylethyl, thiophen-2-ylmethyl, cyclopentylmethyl, 2-hydroxypropyl, 3-hydroxypropyl, 2,3-dihydroxypropyl, 2-hydroxyethyl, 2-ethoxyethyl, 5-methylfuran-2-ylmethyl, cyclopropyl, cyclohexyl, 3-methoxypropyl, 1-hydroxy-4-methylpent-2-yl, 1-(furan-2-yl)ethyl, 5-(dimethylaminomethyl)furan-2-ylmethyl, 5-bromofuran-2-ylmethyl, 5-chlorofuran-2-methyl, 1,3-dihydroxyprop-2-yl, 1,3-dihydroxy-2-methylprop-2-yl, 3-hydroxy-2-methylprop-2-yl, 3-methoxyprop-2-yl, 1-tert-butyl-2-hydroxyethyl, 1-hydroxy-3-methylpent-2-yl, 1,3-dihydroxy-2-hydroxymethylprop-2-yl, 1,3-dihydroxybut-2-yl, 1,2-dimethylpyrrol-5-ylmethyl, 1-methylpyrrol-2-ylmethyl, imidazol-1-ylpropyl, furan-3-ylmethyl, 2,5-dimethylfuran-3-ylmethyl, 3-(methoxycarbonyl)furan-2-ylmethyl, 6-hydroxyhexyl, N-benzylpiperidin-4-yl, N-benzylpyrrolidin-3-yl, 2-phenyloxyethyl, benzyl, morpholin-4-yl, 2-(morpholin-4-yl)ethyl, 4,5-dihydrothiazol-2-yl, piperidin-4-yl, piperidin-4-ylmethyl, 2-methylpropyl, tert-butyl, methyl, tetrahydrofuran-2-ylmethyl, hydroxy, methoxy, ethyl, propyl, 2-fluoroethyl, 2,2,2-trifluoroethyl, 2,2,3,3,3-pentafluoropropyl, 2-methylthioethyl, —(CH₂)₂O(CH₂)₂O(CH₂)₂NH₂, 1-carboxy-3-methylbut-1-yl, 2-carboxyethyl, 3-aminocarbonyl-1-carboxypropyl, 1-carboxy-2-methylbutyl, carboxymethyl, 1-carboxy-2-methylpropyl, 2-phenyl-1-carboxyethyl, 2,3-dimethoxyphenylmethyl, 3,5-dimethoxyphenylmethyl, 3,4-difluorophenylmethyl, 2,4-difluorophenylmethyl, 4-fluorophenylmethyl, 3-difluoromethoxyphenylmethyl, 2,6-dimethoxyphenylmethyl, 2-(methylsulfinyl)ethyl, 2-(methylsulfonyl)ethyl, 2-hydroxyphenyl, 4-chloro-2-hydroxyphenyl, 2-amino-4-oxo-3H-pyrimidin-6-yl, 2-cyanophenyl, 5-amino-1-carboxypentyl, 5-amino-1-aminocarbonylpentyl, 2-(4-methoxyphenyl)ethyl, or guanidino; and
R^{3′} is hydrogen or methyl.

12. The compound of claim 9 wherein R³ is 2-hydroxypyrid-6-yl, 2-chloropyrid-3-yl, 2-thio-[1,3,4]-thiadiazol-2-yl, 5,8-diphenyl-[1,2,4]triazocin-3-yl, 6-ethoxy-benzothiazol-2-yl, 6-fluoro-benzothiazol-2-yl, 3,5-dimethylisoxazol-4-yl, 5-methylisoxazol-3-ylmethyl, pyrimidin-2-yl, 3-methylpyrid-2-yl, 4-methylpyrid-2-yl, 5-methylpyrid-2-yl, 6-methylpyrid-2-yl, 4,6-dimethylpyrid-2-yl, 3-methylpyrid-4-yl, 2-methylpyrid-4-yl, 1,3-dimethylpyrazol-5-yl, 5-methylpyrazol-3-yl, 4-methylpyrimidin-2-yl, 4,6-dimethylpyrimidin-2-yl, 2,4-dimethylpyrimidin-6-yl, pyrazin-2-yl, pyrid-4-yl, pyrid-2-yl, pyrid-3-yl, pyrazol-3-yl, furan-2-ylmethyl, furan-2-ylcarbonyl, 5,6-dimethyl-[1,2,4]-triazin-3-yl, pyrimidin-4-yl, [1,3,4]-thiadiazol-2-yl, thiazol-2-yl, isoxazol-3-yl, cyclopentyl, 1H-pyrimidin-2,4-dione-5-yl, 2-methoxyethyl, cyclobutyl, cyclopropylmethyl, 3-hydroxyprop-2-yl, cyclohexylmethyl, pyrid-2-ylmethyl, pyrid-3-ylmethyl, pyrid-4-ylmethyl, pyrid-4-ylethyl, imidazol-4-ylethyl, thiophen-2-ylmethyl, cyclopentylmethyl, 2-hydroxypropyl, 3-hydroxypropyl, 2,3-dihydroxypropyl, 2-hydroxyethyl, 2-ethoxyethyl, 5-methylfuran-2-ylmethyl, cyclopropyl, cyclohexyl, 3-methoxypropyl, 1-hydroxy-4-methylpent-2-yl, 1-(furan-2-yl)ethyl, 5-(dimethylaminomethyl)furan-2-ylmethyl, 5-bromofuran-2-ylmethyl, 5-chlorofuran-2-methyl, 1,3-dihydroxyprop-2-yl, 1,3-dihydroxy-2-methylprop-2-yl, 3-hydroxy-2-methylprop-2-yl, 3-methoxyprop-2-yl, 1-tert-butyl-2-hydroxyethyl, 1-hydroxy-3-methylpent-2-yl, 1,3-dihydroxy-2-hydroxymethylprop-2-yl, 1,3-dihydroxybut-2-yl, 1,2-dimethylpyrrol-5-ylmethyl, 1-methylpyrrol-2-ylmethyl, imidazol-1-ylpropyl, furan-3-ylmethyl, 2,5-dimethylfuran-3-ylmethyl, 3-(methoxycarbonyl)furan-2-ylmethyl, 6-hydroxyhexyl, N-benzylpiperidin-4-yl, N-benzylpyrrolidin-3-yl, 2-phenyloxyethyl, benzyl, morpholin-4-yl, 2-(morpholin-4-yl)ethyl, 4,5-dihydrothiazol-2-yl, piperidin-4-yl, piperidin-4-ylmethyl, 2-methylpropyl, tert-butyl, methyl, tetrahydrofuran-2-ylmethyl, hydroxy, methoxy, ethyl, propyl, 2-fluoroethyl, 2,2,2-trifluoroethyl, 2,2,3,3,3-pentafluoropropyl, 2-methylthioethyl, —(CH₂)₂O(CH₂)₂O(CH₂)₂NH₂, 1-carboxy-3-methylbut-1-yl, 2-carboxyethyl, 3-aminocarbonyl-1-carboxypropyl, 1-carboxy-2-methylbutyl, carboxymethyl, 1-carboxy-2-methylpropyl, 2-phenyl-1-carboxyethyl, 2,3-dimethoxyphenylmethyl, 3,5-dimethoxyphenylmethyl, 3,4-difluorophenylmethyl, 2,4-difluorophenylmethyl, 4-fluorophenylmethyl, 3-difluoromethoxyphenylmethyl, 2,6-dimethoxyphenylmethyl, 2-(methylsulfinyl)ethyl, 2-(methylsulfonyl)ethyl, 2-hydroxyphenyl, 4-chloro-2-hydroxyphenyl, 2-amino-4-oxo-3H-pyrimidin-6-yl, 2-cyanophenyl, 5-amino-1-carboxypentyl, 5-amino-1-aminocarbonylpentyl, 2-(4-methoxyphenyl)ethyl, or guanidino; and
R3′ is hydrogen or methyl.

13. The compound of claim 1 wherein R³ is 2-hydroxypyrid-6-yl, 2-chloropyrid-3-yl, 2-thio-[1,3,4]-thiadiazol-2-yl, 5,8-diphenyl-[1,2,4]triazocin-3-yl, 6-ethoxy-benzothiazol-2-yl, 6-fluoro-benzothiazol-2-yl, 3,5-dimethylisoxazol-4-yl, 5-methylisoxazol-3-ylmethyl, pyrimidin-2-yl, 3-methylpyrid-2-yl, 4-methylpyrid-2-yl, 5-methylpyrid-2-yl, 6-methylpyrid-2-yl, 4,6-dimethylpyrid-2-yl, 3-methylpyrid-4-yl, 2-methylpyrid-4-yl, 1,3-dimethylpyrazol-5-yl, 5-methylpyrazol-3-yl, 4-methylpyrimidin-2-yl, 4,6-dimethylpyrimidin-2-yl, 2,4-dimethylpyrimidin-6-yl, pyrazin-2-yl, pyrid-4-yl, pyrid-2-yl, pyrid-3-yl, pyrazol-3-yl, furan-2-ylmethyl, furan-2-ylcarbonyl, 5,6-dimethyl-[1,2,4]-triazin-3-yl, pyrimidin-4-yl, [1,3,4]-thiadiazol-2-yl, thiazol-2-yl, isoxazol-3-yl, cyclopentyl, 1H-pyrimidin-2,4-dione-5-yl, 2-methoxyethyl, cyclobutyl, cyclopropylmethyl, 3-hydroxyprop-2-yl, cyclohexylmethyl, pyrid-2-ylmethyl, pyrid-3-ylmethyl, pyrid-4-ylmethyl, pyrid-4-ylethyl, imidazol-4-ylethyl, thiophen-2-ylmethyl, cyclopentylmethyl, 2-hydroxypropyl, 3-hydroxypropyl, 2,3-dihydroxypropyl, 2-hydroxyethyl, 2-ethoxyethyl, 5-methylfuran-2-ylmethyl, cyclopropyl, cyclohexyl, 3-methoxypropyl, 1-hydroxy-4-methylpent-2-yl, 1-(furan-2-yl)ethyl, 5-(dimethylaminomethyl)furan-2-ylmethyl, 5-bromofuran-2-ylmethyl, 5-chlorofuran-2-methyl, 1,3-dihydroxyprop-2-yl, 1,3-dihydroxy-2-methylprop-2-yl, 3-hydroxy-2-methylprop-2-yl, 3-methoxyprop-2-yl, 1-tert-butyl-2-hydroxyethyl, 1-hydroxy-3-methylpent-2-yl, 1,3-dihydroxy-2-hydroxymethylprop-2-yl, 1,3-dihydroxybut-2-yl, 1,2-dimethylpyrrol-5-ylmethyl, 1-methylpyrrol-2-ylmethyl, imidazol-1-ylpropyl, furan-3-ylmethyl, 2,5-dimethylfuran-3-ylmethyl, 3-(methoxycarbonyl)furan-2-ylmethyl, 6-hydroxyhexyl, N-benzylpiperidin-4-yl, N-benzylpyrrolidin-3-yl, 2-phenyloxyethyl, benzyl, morpholin-4-yl, 2-(morpholin-4-yl)ethyl, 4,5-dihydrothiazol-2-yl, piperidin-4-yl, piperidin-4-ylmethyl, 2-methylpropyl, tert-butyl, methyl, tetrahydrofuran-2-ylmethyl, hydroxy, methoxy, ethyl, propyl, 2-fluoroethyl, 2,2,2-trifluoroethyl, 2,2,3,3,3-pentafluoropropyl, 2-methylthioethyl, —(CH₂)₂O(CH₂)₂O(CH₂)₂NH₂, 1-carboxy-3-methylbut-1-yl, 2-carboxyethyl, 3-aminocarbonyl-1-carboxypropyl, 1-carboxy-2-methylbutyl, carboxymethyl, 1-carboxy-2-methylpropyl, 2-phenyl-1-carboxyethyl, 2,3-dimethoxyphenylmethyl, 3,5-dimethoxyphenylmethyl, 3,4-difluorophenylmethyl, 2,4-difluorophenylmethyl, 4-fluorophenylmethyl, 3-difluoromethoxyphenylmethyl, 2,6-dimethoxyphenylmethyl, 2-(methylsulfinyl)ethyl, 2-(methylsulfonyl)ethyl, 2-hydroxyphenyl, 4-chloro-2-hydroxyphenyl, 2-amino-4-oxo-3H-pyrimidin-6-yl, 2-cyanophenyl, 5-amino-1-carboxypentyl, 5-amino-1-aminocarbonylpentyl, 2-(4-methoxyphenyl)ethyl, or guanidino; and
R^{3′} is hydrogen or methyl.

14. The compound of claims 5 wherein R^{3′} is hydrogen.

15. The compound of claims 6 wherein R^{3′} is hydrogen.

16. A compound of Formula I wherein:
R⁴ and R⁵ are trifluoromethyl and are located at the 3- and 5-position of the phenyl ring;
R⁶ and R⁷ are hydrogen;
R¹ is hydrogen, alkyl, or -alkylene-CONR⁸R⁹ where R⁸ and R⁹ together with the nitrogen atom to which they are attached form heterocycloamino;
R² is hydrogen or alkyl; and
R^{3′} is hydrogen.

17. The compound of claim 16 wherein R³ is optionally substituted heteroaryl, optionally substituted heteroaralkyl, hydroxyalkyl, cycloalkyl, cycloalkylalkyl, alkoxyalkyl, —C(O)R¹² where R¹² is optionally substituted heteroaryl or heterocycloalkyl.

18. The compound of claim 17 wherein R³ is furan-2-ylcarbonyl, furan-2-ylmethyl, cyclohexylmethyl, pyrid-2-ylmethyl, pyrid-3-ylmethyl, pyrid-4-ylmethyl, pyrid-4-ylethyl, imidazol-4-ylethyl, thiophen-2-ylmethyl, cyclopentylmethyl, 2-hydroxypropyl, 3-hydroxyprop-2-yl, hydroxypropyl, 2,3-dihydroxypropyl, hydroxyethyl, ethoxyethyl, 5-methylfuran-2-ylmethyl, cyclopropyl, cyclohexyl, 3-methoxypropyl, 1-hydroxy-4-methylpent-2-yl, 1-(furan-2-yl)ethyl, 5-(dimethylaminomethyl)furan-2-ylmethyl, 5-bromofuran-2-ylmethyl, 5-chlorofuran-2-methyl, 1,3-dihydroxyprop-2-yl, 1,3-dihydroxy-2-methylprop-2-yl, 3-hydroxy-2-methylprop-2-yl, 3-methoxyprop-2-yl, 1-tert-butyl-2-hydroxyethyl, 1-hydroxy-3-methylpent-2-yl, 1,3-dihydroxy-2-hydroxymethylprop-2-yl, 1,3-dihydroxybut-2-yl, 1,2-dimethylpyrrol-5-ylmethyl, 1-methylpyrrol-2-ylmethyl, imidazol-1-ylpropyl, furan-3-ylmethyl, 2,5-dimethylfuran-3-ylmethyl, 3-methoxycarbonylfuran-2-ylmethyl, 6-hydroxyhexyl, cyclopentyl, 1H-pyrimidin-2,4-dione-5-yl, 2-methoxyethyl, cyclobutyl, cyclopropylmethyl, furan-2-ylmethyl, or 3-hydroxyprop-2-yl.

19. A compound selected from the group consisting of:
3-(3,5-bis-trifluoromethylphenyl)-4-[(furan-2-ylmethyl)-aminocarbonyl]-2-methyl-1-oxo-1,2,3,4-tetrahydroisoquinoline;
3-(3,5-bis-trifluoromethylphenyl)-4-[(furan-2-ylmethyl)-aminocarbonyl]-2,3-dimethyl-1-oxo-1,2,3,4-tetrahydroisoquinoline;
3-(3,5-bis-trifluoromethylphenyl)-4-[(furan-2-ylmethyl)-aminocarbonyl]-1-oxo-1,2,3,4-tetrahydroisoquinoline;
3-(3,5-bis-trifluoromethylphenyl)-4-[(2-methoxyethyl)-aminocarbonyl]-2-[2-(piperidin-1-ylcarbonyl)ethyl]-1-oxo-1,2,3,4-tetrahydroisoquinoline;
3-(3,5-bis-trifluoromethylphenyl)-4-[(2-methoxyethyl)-aminocarbonyl]-2-methyl-1-oxo-1,2,3,4-tetrahydroisoquinoline;
3-(3,5-bis-trifluoromethylphenyl)-4-[(cyclopentyl)-aminocarbonyl]-1-oxo-1,2,3,4-tetrahydroisoquinoline;
3-(3,5-bis-trifluoromethylphenyl)-4-[(1H-pyrimidin-2,4-dione-5-yl)-aminocarbonyl]-2-methyl-1-oxo-1,2,3,4-tetrahydroisoquinoline;
3-(3,5-bis-trifluoromethylphenyl)-4-{([1,3,4]-thiadiazol-2-yl)-aminocarbonyl}-2-methyl-1-oxo-1,2,3,4-tetrahydroisoquinoline;
3-(3,5-bis-trifluoromethylphenyl)-4-[(2-methoxyethyl)-aminocarbonyl]-1-oxo-1,2,3,4-tetrahydroisoquinoline;
3-(3,5-bis-trifluoromethylphenyl)-4-[(3-hydroxyprop-2-yl)-aminocarbonyl]-2-methyl-1-oxo-1,2,3,4-tetrahydroisoquinoline;
3-(3,5-bis-trifluoromethylphenyl)-4-[(1-hydroxy-3,3-dimethyl-2-butyl)-aminocarbonyl]-2-methyl-1-oxo-1,2,3,4-tetrahydroisoquinoline;
3-(3,5-bis-trifluoromethylphenyl)-4-[(furan-2-ylmethyl)-aminocarbonyl]-2-[2-(4-hydroxypiperidin-1-yl-carbonyl)ethyl]-1-oxo-1,2,3,4-tetrahydroisoquinoline;
3-(3,5-bis-trifluoromethylphenyl)-4-[(cyclopropylmethyl)-aminocarbonyl]-2-methyl-1-oxo-1,2,3,4-tetrahydroisoquinoline;
3-(3,5-bis-trifluoromethylphenyl)-4-[(thiophen-2-ylmethyl)-aminocarbonyl]-2,3-dimethyl-1-oxo-1,2,3,4-tetrahydroisoquinoline;
3-(3,5-bis-trifluoromethylphenyl)-4-[(2-ethoxyethyl)-aminocarbonyl)-2-methyl-1-oxo-1,2,3,4-tetrahydroisoquinoline;
3-(3,5-bis-trfluoromethylphenyl)-4-[(2-fluoroethyl)-aminocarbonyl]-2-methyl-oxo-1,2,3,4-tetrahydroisoquinoline;
3-(3,5-bis-trifluoromethylphenyl)-4-[(2-methoxyethyl)-aminocarbonyl]-2,3-dimethyl-1-oxo-1,2,3,4-tetrahydroisoquinoline;
3-(3,5-bis-trifluoromethylphenyl)-4-[(2-methylthioethyl)-aminocarbonyl]-2-methyl-1-oxo-1,2,3,4-tetrahydroisoquinoline;
3-(3,5-bis-trifluoromethylphenyl)-4-[(3-hydroxypropyl)-aminocarbonyl]-2-methyl-1-oxo-1,2,3,4-tetrahydroisoquinoline;
3-(3,5-bis-trifluoromethylphenyl)-4-[(cyclobutyl)-aminocarbonyl]-2-methyl-1-oxo-1,2,3,4-tetrahydroisoquinoline;
3-(3,5-bis-trifluoromethylphenyl)-4-[(n-propyl)-aminocarbonyl]-2-methyl-1-oxo-1,2,3,4-tetrahydroisoquinoline;
3-(3,5-bis-trifluoromethylphenyl)-4-[(1-furan-2-ylethyl)-aminocarbonyl]-2-methyl-1-oxo-1,2,3,4-tetrahydroisoquinoline;
3-(3,5-bis-trifluoromethylphenyl)-4-[(5-methylfuran-2-yl-methyl)-aminocarbonyl]-2-methyl-1-oxo-1,2,3,4-tetrahydroisoquinoline;
3-(3,5-bis-trifluoromethylphenyl)-4-[(cyclopropylmethyl)-aminocarbonyl]-2,3-dimethyl-1-oxo-1,2,3,4-tetrahydroisoquinoline;
3-(3,5-bis-trifluoromethylphenyl)-4-[(2-hydroxyethyl)-aminocarbonyl]-2-methyl-1-oxo-1,2,3,4-tetrahydroisoquinoline;
3-(3,5-bis-trifluoromethylphenyl)-4-[(cyclopropyl)-aminocarbonyl]-2-methyl-1-oxo-1,2,3,4-tetrahydroisoquinoline;
3-(3,5-bis-trifluoromethylphenyl)-4-[(tetrahydrofuran-2-ylmethyl)-aminocarbonyl]-2-methyl-1-oxo-1,2,3,4-tetrahydroisoquinoline;
3-(3,5-bis-trifluoromethylphenyl)-4-[(5-methylfuran-2-yl-methyl)-aminocarbonyl]-1-oxo-1,2,3,4-tetrahydroisoquinoline;
3-(3,5-bis-trifluoromethylphenyl)-4-[(thiazol-2-yl)-aminocarbonyl]-2-methyl-1-oxo-1,2,3,4-tetrahydroisoquinoline;
3-(3,5-bis-trifluoromethylphenyl)-4-[(pyridin-2-ylmethyl)-aminocarbonyl]-2-methyl-1-oxo-1,2,3,4-tetrahydroisoquinoline;
3-(3,5-bis-trifluoromethylphenyl)-4-[(furan-2-yl-methyl)-aminocarbonyl]-2-(2-piperidin-1-ylcarbonylethyl)-1-oxo-1,2,3,4-tetrahydroisoquinoline;
3-(3,5-bis-trifluoromethylphenyl)-4-[(cyclopentyl)-aminocarbonyl]-2-methyl-1-oxo-1,2,3,4-tetrahydroisoquinoline;
3-(3,5-bis-trifluoromethylphenyl)-4-[(cyclopropylmethyl)-aminocarbonyl]-1-oxo-1,2,3,4-tetrahydroisoquinoline;
3-(3,5-bis-trifluoromethylphenyl)-4-[(furan-2-ylmethyl)-aminocarbonyl]-2-(cyclopropyl)-1-oxo-1,2,3,4-tetrahydroisoquinoline;
3-(3,5-bis-trifluoromethylphenyl)-4-[(thiazol-2-yl)-aminocarbonyl]-1-oxo-1,2,3,4-tetrahydroisoquinoline;
3-(3,5-bis-trifluoromethylphenyl)-4-[(cyclopentyl)-aminocarbonyl]-2-[2-(4-hydroxypiperidin-1-yl-carbonyl)ethyl]-1-oxo-1,2,3,4-tetrahydroisoquinoline;
3-(3,5-bis-trifluoromethylphenyl)-4-[(benzyl)-aminocarbonyl]-2-methyl-1-oxo-1,2,3,4-tetrahydroisoquinoline;
3-(3,5-bis-trifluoromethylphenyl)-4-[(benzyl)-aminocarbonyl]-2-(2-dimethylaminocarbonylethyl)-1-oxo-1,2,3,4-tetrahydroisoquinoline;
3-(3,5-bis-trifluoromethylphenyl)-4-[(cyclopentyl)-aminocarbonyl]-2-(2-dimethylaminocarbonylethyl)-1-oxo-1,2,3,4-tetrahydroisoquinoline;
3-(3,5-bis-trifluoromethylphenyl)-4-[(furan-3-ylmethyl)-aminocarbonyl]-2-methyl-1-oxo-1,2,3,4-tetrahydroisoquinoline;
3-(3,5-bis-trifluoromethylphenyl)-4-[(pyridin-3-ylmethyl)-aminocarbonyl]-2-methyl-1-oxo-1,2,3,4-tetrahydroisoquinoline;
3-(3,5-bis-trifluoromethylphenyl)-4-[(cyclopentyl)-aminocarbonyl]-2-cyclopropyl-1-oxo-1,2,3,4-tetrahydroisoquinoline;
3-(3,5-bis-trifluoromethylphenyl)-4-[(2-methoxyethyl)-aminocarbonyl]-2-cyclopropyl-1-oxo-1,2,3,4-tetrahydroisoquinoline;
3-(3,5-bis-trifluoromethylphenyl)-4-[(2-chloro-pyridin-3-yl)-aminocarbonyl]-2-methyl-1-oxo-1,2,3,4-tetrahydroisoquinoline;
3-(3,5-bis-trifluoromethylphenyl)-4-[(4,5-dihydrothiazol-2-yl)-aminocarbonyl]-1-oxo-1,2,3,4-tetrahydroisoquinoline;
3-(3,5-bis-trifluoromethylphenyl)-4-[(cyclopropylmethyl)-aminocarbonyl]-2-[2-(4-hydroxypiperidin-1-ylcarbonyl)-ethyl]-1-oxo-1,2,3,4-tetrahydroisoquinoline;
3-(3,5-bis-trifluoromethylphenyl)-4-[(4,5-dihydrothiazol-2-yl)-aminocarbonyl]-2-methyl-1-oxo-1,2,3,4-tetrahydroisoquinoline;
3-(3,5-bis-trifluoromethylphenyl)-4-[(2-methoxyethyl)-aminocarbonyl]-2-(2-dimethylaminocarbonylethyl)-1-oxo-1,2,3,4-tetrahydroisoquinoline;
3-(3,5-bis-trifluoromethylphenyl)-4-[(benzyl)-aminocarbonyl]-2-[2-(morpholin-4-ylcarbonyl)-ethyl]-1-oxo-1,2,3,4-tetrahydroisoquinoline;
3-(3,5-bis-trifluoromethylphenyl)-4-[(benzyl)-aminocarbonyl]-2-[2-(4-hydroxypiperidin-1-yl-carbonyl)-ethyl]-1-oxo-1,2,3,4-tetrahydroisoquinoline;
3-(3,5-bis-trifluoromethylphenyl)-4-[(cyclopentyl)-aminocarbonyl]-2-[2-(4-acetylpiperazin-1-yl-carbonyl)ethyl]-1-oxo-1,2,3,4-tetrahydroisoquinoline;
3-(3,5-bis-trifluoromethylphenyl)-4-[(cyclopentyl)-aminocarbonyl]-2-(2-morpholin-4-ylethyl)-1-oxo-1,2,3,4-tetrahydroisoquinoline;
3-(3,5-bis-trifluoromethylphenyl)-4-[(benzyl)-aminocarbonyl]-2-{(CH₂)₂C(O){N(CH₃)—[(CH₂)₂OCH₃]}}-1-oxo-1,2,3,4-tetrahydroisoquinoline;
3-(3,5-bis-trifluoromethylphenyl)-4-[(furan-2-ylmethyl)-aminocarbonyl]-2-(2-propyl)-1-oxo-1,2,3,4-tetrahydroisoquinoline;
3-(3,5-bis-trifluoromethylphenyl)-4-[(cyclopropylmethyl)-aminocarbonyl]-2-(2-dimethylaminocarbonylethyl)-1-oxo-1,2,3,4-tetrahydroisoquinoline;
3-(3,5-bis-trifluoromethylphenyl)-4-[(furan-2-ylmethyl)-aminocarbonyl]-2-(2-dimethylaminocarbonylethyl)-1-oxo-1,2,3,4-tetrahydroisoquinoline;
3-(3,5-bis-trifluoromethylphenyl)-4-[(benzyl)-aminocarbonyl]-1-oxo-1,2,3,4-tetrahydroisoquinoline;
3-(3,5-bis-trifluoromethylphenyl)-4-[(cyclopentyl)-aminocarbonyl]-2-(2-piperidin-1-ylcarbonylethyl)-1-oxo-1,2,3,4-tetrahydroisoquinoline;
3-(3,5-bis-trifluoromethylphenyl)-4-[(cyclopentyl)-aminocarbonyl]-2-(2-propyl)-1-oxo-1,2,3,4-tetrahydroisoquinoline;
3-(3,5-bis-trifluoromethylphenyl)-4-[(pyridin-4-ylmethyl)-aminocarbonyl]-2-methyl-1-oxo-1,2,3,4-tetrahydroisoquinoline;
3-(3,5-bis-trifluoromethylphenyl)-4-[(cyclopentyl)-aminocarbonyl]-2-(2-methoxyethyl)-1-oxo-1,2,3,4-tetrahydroisoquinoline;
3-(3,5-bis-trifluoromethylphenyl)-4-[(cyclopentylmethyl)-aminocarbonyl]-2-methyl-1-oxo-1,2,3,4-tetrahydroisoquinoline;
3-(3,5-bis-trifluoromethylphenyl)-4-[(benzyl)-aminocarbonyl]-2-{(CH₂)₂C(O){N(CH₃)—[(CH₂)₃CH₃]}}-1-oxo-1,2,3,4-tetrahydroisoquinoline;
3-(3,5-bis-trifluoromethylphenyl)-4-[(2-methoxyethyl)-aminocarbonyl]-2-(2-methoxyethyl)-1-oxo-1,2,3,4-tetrahydroisoquinoline;
3-(3,5-bis-trifluoromethylphenyl)-4-[(furan-2-ylmethyl)-aminocarbonyl]-2-(morpholin-4-ylethyl)-1-oxo-1,2,3,4-tetrahydroisoquinoline;
3-(3,5-bis-trifluoromethylphenyl)-4-[(benzyl)-aminocarbonyl]-2-{(CH₂)₂CO{N(CH₃)(benzyl)}}-1-oxo-1,2,3,4-tetrahydroisoquinoline;
3-(3,5-bis-trifluoromethylphenyl)-4-[(benzyl)-aminocarbonyl]-2-{(CH₂)₂CO{N(CH₃)[2-(3,4-dimethyoxyphenyl)ethyl]}}-1-oxo-1,2,3,4-tetrahydroisoquinoline;
3-(3,5-bis-trifluoromethylphenyl)-4-[(2-imidazol-4-ylethyl)-aminocarbonyl]-2-1-oxo-1,2,3,4-tetrahydroisoquinoline;
3-(3,5-bis-trifluoromethylphenyl)-4-[(cyclopropylmethyl)-aminocarbonyl]-2-[2-(4-acetylpiperazin-1-yl-carbonyl)ethyl]-1-oxo-1,2,3,4-tetrahydroisoquinoline;
3-(3,5-bis-trifluoromethylphenyl)-4-[(2-methoxyethyl)-aminocarbonyl]-2-(furan-2-ylmethyl)-1-oxo-1,2,3,4-tetrahydroisoquinoline;
3-(3,5-bis-trifluoromethylphenyl)-4-[(2-methoxyethyl)-aminocarbonyl]-2-[2-(4-acetylpiperazin-1-yl-carbonyl)ethyl]-1-oxo-1,2,3,4-tetrahydroisoquinoline;
3-(3,5-bis-trifluoromethylphenyl)-4-[(furan-3-ylmethyl)-aminocarbonyl]-1-oxo-1,2,3,4-tetrahydroisoquinoline;
3-(3,5-bis-trifluoromethylphenyl)-4-[(morpholin-4-yl)-aminocarbonyl]-1-oxo-1,2,3,4-tetrahydroisoquinoline;
3-(3,5-bis-trifluoromethylphenyl)-4-[(2-methoxyethyl)-aminocarbonyl]-2-(2-morpholin-4-ylethyl)-1-oxo-1,2,3,4-tetrahydroisoquinoline;
3-(3,5-bis-trifluoromethylphenyl)-4-[(2-hydroxypyridin-6-yl)-aminocarbonyl]-2-methyl-1-oxo-1,2,3,4-tetrahydroisoquinoline;
3-(3,5-bis-trifluoromethylphenyl)-4-[(2-pyridin-4-ylethyl)-aminocarbonyl]-2-methyl-1-oxo-1,2,3,4-tetrahydroisoquinoline;
3-(3,5-bis-trifluoromethylphenyl)-4-[(benzyl)-aminocarbonyl]-2-[2-(4-formylpiperazin-1-yl-carbonyl)ethyl]-1-oxo-1,2,3,4-tetrahydroisoquinoline;
3-(3,5-bis-trifluoromethylphenyl)-4-[(2-morpholin-4-ylethyl)-aminocarbonyl]-1-oxo-1,2,3,4-tetrahydroisoquinoline;
3-(3,5-bis-trifluoromethylphenyl)-4-[(2-methoxyethyl)-aminocarbonyl]-2-(2-propyl)-1-oxo-1,2,3,4-tetrahydroisoquinoline;
3-(3,5-bis-trifluoromethylphenyl)-4-[(cyclopentyl)-aminocarbonyl]-2-(furan-2-ylmethyl)-1-oxo-1,2,3,4-tetrahydroisoquinoline;
3-(3,5-bis-trifluoromethylphenyl)-4-[(piperidin-4-ylmethyl)-aminocarbonyl]-2-methyl-1-oxo-1,2,3,4-tetrahydroisoquinoline; 3-(3,5-bis-trifluoromethylphenyl)-4-[(5-dimethylaminofuran-2-ylmethyl)-aminocarbonyl]-2-methyl-1-oxo-1,2,3,4-tetrahydroisoquinoline;
3-(3,5-bis-trifluoromethylphenyl)-4-[(5-bromofuran-2-y-methyl)-aminocarbonyl]-2-methyl-1-oxo-1,2,3,4-tetrahydroisoquinoline;
3-(3,5-bis-trifluoromethylphenyl)-4-[(hydroxy)-aminocarbonyl]-2-methyl-1-oxo-1,2,3,4-tetrahydroisoquinoline;
3-(3,5-bis-trifluoromethylphenyl)-4-[(isoxazol-3-yl)-aminocarbonyl]-2-methyl-1-oxo-1,2,3,4-tetrahydroisoquinoline;
3-(3,5-bis-trifluoromethylphenyl)-4-[(1-carboxy-2-methyl-1-butyl)-aminocarbonyl]-2-methyl-1-oxo-1,2,3,4-tetrahydroisoquinoline;
3-(3,5-bis-trifluoromethylphenyl)-4-[(isoxazol-3-yl)-aminocarbonyl]-1-oxo-1,2,3,4-tetrahydroisoquinoline;
3-(3,5-bis-trifluoromethylphenyl)-4-[(2,4-dimethylpyrid-6-yl)-aminocarbonyl]-2-methyl-1-oxo-1,2,3,4-tetrahydroisoquinoline;
3-(3,5-bis-trifluoromethylphenyl)-4-[(pyrazol-3-yl)-aminocarbonyl]-2-methyl-1-oxo-1,2,3,4-tetrahydroisoquinoline;
3-(3,5-bis-trifluoromethylphenyl)-4-[(4-methylpyrimidin-2-yl)-aminocarbonyl]-2-methyl-1-oxo-1,2,3,4-tetrahydroisoquinoline;
3-(3,5-bis-trifluoromethylphenyl)-4-[(4,6-dimethylpyrimidin-2-yl)-aminocarbonyl]-2-methyl-1-oxo-1,2,3,4-tetrahydroisoquinoline;
3-(3,5-bis-trifluoromethylphenyl)-4-[(2,4-dimethylpyrimidin-6-yl)-aminocarbonyl]-2-methyl-1-oxo-1,2,3,4-tetrahydroisoquinoline;
3-(3,5-bis-trifluoromethylphenyl)-4-[(pyrazin-2-yl)-aminocarbonyl]-2-methyl-1-oxo-1,2,3,4-tetrahydroisoquinoline;
3-(3,5-bis-trifluoromethylphenyl)-4-[(pyridin-4-yl)-aminocarbonyl]-2-methyl-1-oxo-1,2,3,4-tetrahydroisoquinoline;
3-(3,5-bis-trifluoromethylphenyl)-4-[(pyridin-2-yl)-aminocarbonyl]-2-methyl-1-oxo-1,2,3,4-tetrahydroisoquinoline;
3-(3,5-bis-trifluoromethylphenyl)-4-[(pyridin-3-yl)-aminocarbonyl]-2-methyl-1-oxo-1,2,3,4-tetrahydroisoquinoline;
3-(3,5-bis-trifluoromethylphenyl)-4-[(5,6-dimethyl-[1,2,4]triazin-3-yl)-aminocarbonyl]-2-methyl-1-oxo-1,2,3,4-tetrahydroisoquinoline;
3-(3,5-bis-trifluoromethylphenyl)-4-[(5,6-dimethyl-[1,2,4]triazin-3-yl)-aminocarbonyl]-1-oxo-1,2,3,4-tetrahydroisoquinoline;
3-(3,5-bis-trifluoromethylphenyl)-4-[(pyrimidin-4-yl)-aminocarbonyl]-2-methyl-1-oxo-1,2,3,4-tetrahydroisoquinoline;
3-(3,5-bis-trifluoromethylphenyl)-4-[(furan-2-ylcarbonyl)-aminocarbonyl]-2-methyl-1-oxo-1,2,3,4-tetrahydroisoquinoline;
3-(3,5-bis-trifluoromethylphenyl)-4-[(2-methylpyrid-4-yl)-aminocarbonyl]-2-methyl-1-oxo-1,2,3,4-tetrahydroisoquinoline;
3-(3,5-bis-trifluoromethylphenyl)-4-[(2-methylpyrid-6-yl)-aminocarbonyl]-2-methyl-1-oxo-1,2,3,4-tetrahydroisoquinoline;
3-(3,5-bis-trifluoromethylphenyl)-4-[(3-methylpyrid-4-yl)-aminocarbonyl]-2-methyl-1-oxo-1,2,3,4-tetrahydroisoquinoline;
3-(3,5-bis-trifluoromethylphenyl)-4-[(3-methylpyrid-6-yl)-aminocarbonyl]-2-methyl-1-oxo-1,2,3,4-tetrahydroisoquinoline;
3-(3,5-bis-trifluoromethylphenyl)-4-[(4-methylpyrid-2-yl)-aminocarbonyl]-2-methyl-1-oxo-1,2,3,4-tetrahydroisoquinoline;
3-(3,5-bis-trifluoromethylphenyl)-4-[(pyrimidin-2-yl)-aminocarbonyl]-2-methyl-1-oxo-1,2,3,4-tetrahydroisoquinoline;
3-(3,5-bis-trifluoromethylphenyl)-4-{[2-(4-methoxyphenyl)ethyl]-aminocarbonyl}-2-(ethoxycarbonylethyl)-1-oxo-1,2,3,4-tetrahydroisoquinoline;
3-(3,5-bis-trifluoromethylphenyl)-4-[(N-benzylpiperidin-4-yl)-aminocarbonyl]-2-(aminocarbonylethyl)-1-oxo-1,2,3,4-tetrahydroisoquinoline;
3-(3,5-bis-trifluoromethylphenyl)-4-[(N-benzylpyrrolidin-3-yl)-aminocarbonyl]-2-(aminocarbonylethyl)-1-oxo-1,2,3,4-tetrahydroisoquinoline;
3-(3,5-bis-trifluoromethylphenyl)-4-[(2-phenoxyethyl)-aminocarbonyl]-2-(aminocarbonylethyl)-1oxo-1,2,3,4-tetrahydroisoquinoline;
3-(3,5-bis-trifluoromethylphenyl)-4-[(benzyl)-aminocarbonyl]-2-{[2-(3-methoxyphenyl)ethyl]aminocarbonylethyl}-1-oxo-1,2,3,4-tetrahydroisoquinoline;
3-(3,5-bis-trifluoromethylphenyl)-4-[(benzyl)-aminocarbonyl]-2-(tetrahydrofuran-2-ylmethyl)-1-oxo-1,2,3,4-tetrahydroisoquinoline;
3-(3,5-bis-trifluoromethylphenyl)-4-[(benzyl)-aminocarbonyl]-2-[(4-methylpiperidin-1-yl)carbonylethyl]-1-oxo-1,2,3,4-tetrahydroisoquinoline;
3-(3,5-bis-trifluoromethylphenyl)-4-[(benzyl)-aminocarbonyl]-2-(thiomorpholin-4-ylcarbonylethyl)-1-oxo-1,2,3,4-tetrahydroisoquinoline;
3-(3,5-bis-trifluoromethylphenyl)-4-[(2-methoxyethyl)-aminocarbonyl]-2-[2-(3-methoxyphenyl)ethylaminocarbonylethyl]-1-oxo-1,2,3,4-tetrahydroisoquinoline;
3-(3,5-bis-trifluoromethylphenyl)-4-[(furan-2-ylmethyl)-aminocarbonyl]-2-(4-acetylpiperazin-1-ylcarbonylethyl)-1-oxo-1,2,3,4-tetrahydroisoquinoline;
3-(3,5-bis-trifluoromethylphenyl)-4-[(furan-2-ylmethyl)-aminocarbonyl]-2-(2-methoxyethyl)-1-oxo-1,2,3,4-tetrahydroisoquinoline;
3-(3,5-bis-trifluoromethylphenyl)-4-[(cyclohexylmethyl)-aminocarbonyl]-1-oxo-1,2,3,4-tetrahydroisoquinoline;
3-(3,5-bis-trifluoromethylphenyl)-4-[(2-methoxyethyl)-aminocarbonyl]-2-(4-methoxyphenylmethyl)-1-oxo-1,2,3,4-tetrahydroisoquinoline;
3-(3,5-bis-trifluoromethylphenyl)-4-[(cyclopropylmethyl)-aminocarbonyl]-2-(4-methoxyphenylmethyl)-1-oxo-1,2,3,4-tetrahydroisoquinoline;
3-(3,5-bis-trifluoromethylphenyl)-4-[(2,3-dihydroxypropyl)-aminocarbonyl]-2-methyl-1-oxo-1,2,3,4-tetrahydroisoquinoline;
3-(3,5-bis-trifluoromethylphenyl)-4-[(methoxy)-aminocarbonyl]-2-methyl-1-oxo-1,2,3,4-tetrahydroisoquinoline;
3-(3,5-bis-trifluoromethylphenyl)-4-[(cyclopentyl)-aminocarbonyl]-2-(methoxycarbonylpentyl)-1-oxo-1,2,3,4-tetrahydroisoquinoline;
3-(3,5-bis-trifluoromethylphenyl)-4-[(2-aminoethyloxyethyloxyethyl)-aminocarbonyl]-2-methyl-1-oxo-1,2,3,4-tetrahydroisoquinoline;
3-(3,5-bis-trifluoromethylphenyl)-4-[(2-carboxy-3-methylbutyl)-aminocarbonyl]-2-methyl-1-oxo-1,2,3,4-tetrahydroisoquinoline;
3-(3,5-bis-trifluoromethylphenyl)-4-{[2-hydroxy-1,1-(dihydroxymethyl)ethyl]-aminocarbonyl}-2-methyl-1-oxo-1,2,3,4-tetrahydroisoquinoline;
3-(3,5-bis-trifluoromethylphenyl)-4-[(3-aminocarbonyl-1-carboxypropyl)-aminocarbonyl]-2-methyl-1-oxo-1,2,3,4-tetrahydroisoquinoline;
3-(3,5-bis-trifluoromethylphenyl)-4-[(1-carboxy-2-phenylethyl)-aminocarbonyl]-2-methyl-1-oxo-1,2,3,4-tetrahydroisoquinoline;
3-(3,5-bis-trifluoromethylphenyl)-4-[(N-methylpyrrol-2-ylmethyl)-aminocarbonyl]-2-methyl-1-oxo-1,2,3,4-tetrahydroisoquinoline;
3-(3,5-bis-trifluoromethylphenyl)-4-[(1,2-dimethylpyrrol-5-ylmethyl)-aminocarbonyl]-1-oxo-1,2,3,4-tetrahydroisoquinoline;
3-(3,5-bis-trifluoromethylphenyl)-4-[(2,3-dimethoxyphenylmethyl)-aminocarbonyl]-2-methyl-1-oxo-1,2,3,4-tetrahydroisoquinoline;
3-(3,5-bis-trifluoromethylphenyl)-4-[(2,4-difluorophenylmethyl)-aminocarbonyl]-2-methyl-1-oxo-1,2,3,4-tetrahydroisoquinoline;
3-(3,5-bis-trifluoromethylphenyl)-4-{[3-(difluoromethoxy)phenylmethyl]-aminocarbonyl}-2-methyl-1-oxo-1,2,3,4-tetrahydroisoquinoline;
3-(3,5-bis-trifluoromethylphenyl)-4-[(imidazol-1-ylpropyl)-aminocarbonyl]-2-methyl-1-oxo-1,2,3,4-tetrahydroisoquinoline;
3-(3,5-bis-trifluoromethylphenyl)-4-{[2-(methylsulfinyl)ethyl]-aminocarbonyl}-2-methyl-1-oxo-1,2,3,4-tetrahydroisoquinoline;
3-(3,5-bis-trifluoromethylphenyl)-4-[(methylsulfonylethyl)-aminocarbonyl]-2-methyl-1-oxo-1,2,3,4-tetrahydroisoquinoline;
3-(3,5-bis-trifluoromethylphenyl)-4-[(2-amino-4-oxo-3H-pyrimidin-6-yl)-aminocarbonyl]-2-methyl-1-oxo-1,2,3,4-tetrahydroisoquinoline;
3-(3,5-bis-trifluoromethylphenyl)-4-[(4-chloro-2-hydroxyphenyl)-aminocarbonyl]-1-oxo-1,2,3,4-tetrahydroisoquinoline;
3-(3,5-bis-trifluoromethylphenyl)-4-[(2-cyanophenyl)-aminocarbonyl]-1-oxo-1,2,3,4-tetrahydroisoquinoline;
3-(3,5-bis-trifluoromethylphenyl)-4-[(3-methoxycarbonylfuran-2-ylmethyl)-aminocarbonyl]-2-methyl-1-oxo-1,2,3,4-tetrahydroisoquinoline;
3-(3,5-bis-trifluoromethylphenyl)-4-{(5-mercapto-[1,3,4]-thiadiazol-2-yl)-aminocarbonyl}-1-oxo-1,2,3,4-tetrahydroisoquinoline;
3-(3,5-bis-trifluoromethylphenyl)-4-[(5-amino-1-carboxypentyl)-aminocarbonyl]-2-methyl-1-oxo-1,2,3,4-tetrahydroisoquinoline;
3-(3,5-bis-trifluoromethylphenyl)-4-[(5-amino-1-aminocarbonylpentyl)-aminocarbonyl]-2-methyl-1-oxo-1,2,3,4-tetrahydroisoquinoline;
3-(3,5-bis-trifluoromethylphenyl)-4-[(5,8-diphenyl-[1,2,4]triazocin-3-yl)-aminocarbonyl]-2-methyl-1-oxo-1,2,3,4-tetrahydroisoquinoline;
3-(3,5-bis-trifluoromethylphenyl)-4-[(6-ethoxybenzothiazol-2-yl)-aminocarbonyl]-2-methyl-1-oxo-1,2,3,4-tetrahydroisoquinoline;
3-(3,5-bis-trifluoromethylphenyl)-4-[(guanidino)-aminocarbonyl]-2-methyl-1-oxo-1,2,3,4-tetrahydroisoquinoline;
3-(3,5-bis-trifluoromethylphenyl)-4-[(3,5-dimethylisoxazol-4-yl)-aminocarbonyl]-2-methyl-1-oxo-1,2,3,4-tetrahydroisoquinoline;
3-(3,5-bis-trifluoromethylphenyl)-4-[(piperidin-4-yl)-aminocarbonyl]-2-methyl-1-oxo-1,2,3,4-tetrahydroisoquinoline;
3-(3,5-bis-trifluoromethylphenyl)-4-[(1,3-pyrazol-5-yl)-aminocarbonyl]-2-methyl-1-oxo-1,2,3,4-tetrahydroisoquinoline;
3-(3-trifluoromethylthiophenyl)-4-[(furan-2-ylmethyl)-aminocarbonyl]-2-methyl-1-oxo-1,2,3,4-tetrahydroisoquinoline;
3-(3-trifluoromethylthiophenyl)-4-[(2-methoxyethyl)-aminocarbonyl]-2-methyl-1-oxo-1,2,3,4-tetrahydroisoquinoline;
3-(3-trifluoromethylthiophenyl)-4-[(cyclopentyl)-aminocarbonyl]-2-methyl-1-oxo-1,2,3,4-tetrahydroisoquinoline;
3-(3,5-dimethylphenyl)-4-[(furan-2-ylmethyl)-aminocarbonyl]-2-methyl-1-oxo-1,2,3,4-tetrahydroisoquinoline;
3-(3,5-dimethylphenyl)-4-[(cyclopentyl)-aminocarbonyl]-2-methyl-1-oxo-1,2,3,4-tetrahydroisoquinoline;
3-(3,5-dimethylphenyl)-4-[(cyclopropylmethyl)-aminocarbonyl]-2-hydroxy-1-oxo-1,2,3,4-tetrahydroisoquinoline;
3-(3,5-dichlorophenyl)-4-[(2-methoxyethyl)-aminocarbonyl]-2-methyl-1-oxo-1,2,3,4-tetrahydroisoquinoline;
3-(3-trifluoromethylphenyl)-4-[(furan-2-ylmethyl)-aminocarbonyl]-2-methyl-1-oxo-1,2,3,4-tetrahydroisoquinoline;
3-(3-trifluoromethylphenyl)-4-[(cyclopentyl)-aminocarbonyl]-2-methyl-1-oxo-1,2,3,4-tetrahydroisoquinoline;
3-(3-trifluoromethylphenyl)-4-[(2-methoxyethyl)-aminocarbonyl]-2-methyl-1-oxo-1,2,3,4-tetrahydroisoquinoline;
3-(3-trifluoromethoxyphenyl)-4-[(2-methoxyethyl)-aminocarbonyl]-2-methyl-1-oxo-1,2,3,4-tetrahydroisoquinoline;
3-(3-trifluoromethoxyphenyl)-4-[(furan-2-ylmethyl)-aminocarbonyl]-2-methyl-1-oxo-1,2,3,4-tetrahydroisoquinoline;
3-(3-trifluoromethoxyphenyl)-4-[(thiazol-2-yl)-aminocarbonyl]-2-methyl-1-oxo-1,2,3,4-tetrahydroisoquinoline;
3-(2,4-bis-trifluoromethylphenyl)-4-[(thiazol-2-yl)-aminocarbonyl]-2-methyl-1-oxo-1,2,3,4-tetrahydroisoquinoline;
3-(2,4-bis-trifluoromethylphenyl)-4-[(thiophen-2-ylmethyl)-aminocarbonyl]-2-methyl-1-oxo-1,2,3,4-tetrahydroisoquinoline;
3-(3,5-dibromophenyl)-4-[(furan-2-ylmethyl)-aminocarbonyl]-2-methyl-1-oxo-1,2,3,4-tetrahydroisoquinoline;
3-phenyl-4-[(2,4-dimethylpyrid-6-yl)-aminocarbonyl]-2-methyl-1-oxo-1,2,3,4-tetrahydroisoquinoline;
3-phenyl-4-[(pyrazol-3-yl)-aminocarbonyl]-2-methyl-1-oxo-1,2,3,4-tetrahydroisoquinoline;
3-phenyl-4-[(2-methoxyethyl)-aminocarbonyl]-1-oxo-1,2,3,4-tetrahydroisoquinoline;
3-(3,5-bis-trifluoromethylphenyl)-4-[(furan-2-ylmethyl)-aminocarbonyl]-7-methoxy-2-methyl-1-oxo-1,2,3,4-tetrahydroisoquinoline;
3-(3,5-bis-trifluoromethylphenyl)-4-[(furan-2-ylmethyl)-aminocarbonyl]-6,7-dimethoxy-1-oxo-1,2,3,4-tetrahydroisoquinoline;
3-(3,5-bis-trifluoromethylphenyl)-4-[(pyridin-2-ylmethyl)-aminocarbonyl]-2,7-dimethyl-1-oxo-1,2,3,4-tetrahydroisoquinoline;
3-(3,5-bis-trifluoromethylphenyl)-4-[(furan-2-ylmethyl)-aminocarbonyl]-2,7-dimethyl-1-oxo-1,2,3,4-tetrahydroisoquinoline;
3-(3,5-bis-trifluoromethylphenyl)-4-[(thiazol-2-ylmethyl)-aminocarbonyl]-2,7-dimethyl-1-oxo-1,2,3,4-tetrahydroisoquinoline;
3-(3,5-bis-trifluoromethylphenyl)-4-[(furan-2-ylmethyl)-aminocarbonyl]-6,7-dimethoxy-2-hydroxy-1-oxo-1,2,3,4-tetrahydroisoquinoline;
3-(3,5-bis-trifluoromethylphenyl)-4-[(2-methoxyethyl)-aminocarbonyl]-2,7-dimethyl-1-oxo-1,2,3,4-tetrahydroisoquinoline;
3-(3,5-bis-trifluoromethylphenyl)-4-[(2-methoxyethyl)-aminocarbonyl]-7-methoxy-2-methyl-1-oxo-1,2,3,4-tetrahydroisoquinoline;
3-(3,5-bis-trifluoromethylphenyl)-4-[(cyclopropylmethyl)-aminocarbonyl]-2,6-dimethyl-1-oxo-1,2,3,4-tetrahydroisoquinoline;
3-(3,5-bis-trifluoromethylphenyl)-4-[(furan-2-ylmethyl)-aminocarbonyl]-6,7-dimethoxy-2-methyl-1-oxo-1,2,3,4-tetrahydroisoquinoline;
3-(3,5-bis-trifluoromethylphenyl)-4-[(thiazol-2-yl)-aminocarbonyl]-6,7-dimethoxy-2-methyl-1-oxo-1,2,3,4-tetrahydroisoquinoline;
3-(3,5-bis-trifluoromethylphenyl)-4-[(cyclopropylmethyl)-aminocarbonyl]-6,7-dimethoxy-1-oxo-1,2,3,4-tetrahydroisoquinoline;
3-(3,5-bis-trifluoromethylphenyl)-4-[(pyrid-4-ylmethyl)-aminocarbonyl]-7-methoxy-2-methyl-1-oxo-1,2,3,4-tetrahydroisoquinoline;
3-(3,5-bis-trifluoromethylphenyl)-4-[(piperidin-4-ylmethyl)-aminocarbonyl]-7-methoxy-2-methyl-1-oxo-1,2,3,4-tetrahydroisoquinoline;
3-(3,5-bis-trifluoromethylphenyl)-4-[(furan-2-ylmethyl)-aminocarbonyl]-2,6-dimethyl-1-oxo-1,2,3,4-tetrahydroisoquinoline;
3-(3,5-bis-trifluoromethylphenyl)-4-[(4,5-dihydro-thiazol-2-yl)-aminocarbonyl]-2,6-dimethyl-1-oxo-1,2,3,4-tetrahydroisoquinoline;
3-(3,5-bis-trifluoromethylphenyl)-4-[(cyclopropylmethyl)-aminocarbonyl]-6-chloro-2-methyl-1-oxo-1,2,3,4-tetrahydroisoquinoline;
3-(3,5-bis-trifluoromethylphenyl)-4-[(cyclopentyl)-aminocarbonyl]-7-chloro-2-methyl-1-oxo-1,2,3,4-tetrahydroisoquinoline;
3-(3,5-bis-trifluoromethylphenyl)-4-[(5-methylfuran-2-ylmethyl)-aminocarbonyl]-7-chloro-2-methyl-1-oxo-1,2,3,4-tetrahydroisoquinoline;
3-(3,5-bis-trifluoromethylphenyl)-4-[(3,5-dimethylmorpholin-4-yl)-carbonyl]-2-(aminocarbonylethyl)-1-oxo-1,2,3,4-tetrahydroisoquinoline;
3-(3,5-bis-trifluoromethylphenyl)-4-[(4-acetylpiperazin-1-yl)-carbonyl]-2-(aminocarbonylethyl)-1-oxo-1,2,3,4-tetrahydroisoquinoline;
3-(3,5-bis-trifluoromethylphenyl)-4-[(4-acetylpiperazin-1-yl)-carbonyl]-2-methyl-1-oxo-1,2,3,4-tetrahydroisoquinoline;
3-(3,5-bis-trifluoromethylphenyl)-4-[(4-acetylpiperazin-1-yl)-carbonyl]-2-(2-methoxyethyl)-1-oxo-1,2,3,4-tetrahydroisoquinoline;
3-(3,5-bis-trifluoromethylphenyl)-4-[(piperazin-1-yl)-carbonyl]-2-methyl-1-oxo-1,2,3,4-tetrahydroisoquinoline;
3-(3,5-bis-trfluoromethylphenyl)-4-[(morpholin-4-yl)-carbonyl]-2-methyl-1-oxo-1,2,3,4-tetrahydroisoquinoline;
3-(3,5-bis-trifluoromethylphenyl)-6,7-dimethoxy-4-[(piperazin-1-yl)-carbonyl]-2-methyl-1-oxo-1,2,3,4-tetrahydroisoquinoline;
3-(3,5-bis-trifluoromethylphenyl)-6,7-dimethoxy-4-[(morpholin-4-yl)-carbonyl]-2-methyl-1-oxo-1,2,3,4-tetrahydroisoquinoline;
3-(3,5-bis-trifluoromethylphenyl)-4-{[N-methyl-N-(2-pyrid-4-ylethyl)]-aminocarbonyl}-2-methyl-1-oxo-1,2,3,4-tetrahydroisoquinoline;
3-(3,5-bis-trifluoromethylphenyl)-4-{[N-methyl-N-(furan-2-ylmethyl)]-aminocarbonyl}-2-methyl-1-oxo-1,2,3,4-tetrahydroisoquinoline; or
3-(3,5-bis-trifluoromethylphenyl)-4-[(3-aminopyrazol-1-yl)-carbonyl]-2-methyl-1-oxo-1,2,3,4-tetrahydroisoquinoline;
or a pharmaceutically acceptable salt thereof.

20. A method of treating a disorder responsive to the induction of apoptosis in an animal suffering said disorder, comprising administering to said animal a pharmaceutical composition comprising a therapeutically effective amount of a compound of claim 1 and a pharmaceutically acceptable excipient.

21. The method of claim 20 wherein the disease is a cancer, autoimmune disease, rheumatoid arthritis, inflammatory bowel disease, or psoriasis.

22. The method of claim 21 wherein the disease is a cancer and is selected from the group consisting of Hodgkin's disease, non-Hodgkin's lymphoma, acute and chronic lymphocytic leukemias, multiple myeloma, neuroblastoma, breast carcinoma, ovarian carcinoma, lung carcinoma, Wilms' tumor, cervical carcinoma, testicular carcinoma, soft-tissue sarcoma, chronic lymphocytic leukemia, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head and neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer and prostatic carcinoma, and the animal is a human.

23. A method of treating cancer in an animal which method comprises administering to said animal a pharmaceutical composition comprising a therapeutically effective amount of a compound of claim 1 and a pharmaceutically acceptable excipient in combination with radiation therapy and optionally in combination with one or more chemotherapeutic compound(s) independently selected from an estrogen receptor modulator, an androgen receptor modulator, retinoid receptor modulator, a cytotoxic agent, another antiproliferative agent, a prenyl-protein transferase inhibitor, an HMG-CoA reductase inhibitor, an HIV protease inhibitor, a reverse transcriptase inhibitor, or an angiogenesis inhibitor.

24. The method of claim 23 wherein the chemotherapeutic compound(s) is independently selected from Taxol®, Taxotere®, epothilone A, epothilone B, desoxyepothilone A, desoxyepothilone B or their derivatives); epidophyllotoxin; procarbazine; mitoxantrone; the mitomycins, discodermolide, podophyllotoxins. doxorubicin, carminomycin, daunorubicin, aminopterin, methotrexate, methopterin, dichloro-methotrexate, mitomycin C, porfiromycin, Herceptin®, Rituxan®, 5-fluorouracil, 6-mercaptopurine, gemcitabine, cytosine arabinoside, colchicines, etoposide, etoposide phosphate or teniposide, melphalan, vinblastine, vincristine, leurosidine, vindesine, leurosine, paclitaxel, estramustine, cisplatin, carboplatin, cyclophosphamide, bleomycin, tamoxifen, ifosamide, melphalan, hexamethyl melamine, thiotepa, cytarabin, idatrexate, trimetrexate, dacarbazine, L-asparaginase, camptothecin, CPT-1 1, topotecan, ara-C, bicalutamide, flutamide, leuprolide, pyridobenzoindole derivatives, interferons and interleukins.

25. A pharmaceutical composition comprising a therapeutically effective amount of a compound of claim 1 and a pharmaceutically acceptable excipient.
